SEQUENCE LISTING (99 sequences)

SEQ ID NO: 1 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 238 aa, Aequorea victoria
MSKGEELFTGVVPILVELDGDVNGHKFSVSGEGEGDATYGKLTLKFICTTGKLPVPWPTLVTTFSWGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - protein, 238 aa, Aequorea victoria
MSKGEELFTGVVPILVELDGDVNGHKFSVSGEGEGDATYGKLTLKFICTTGKLPVPWPTLVTTFSHGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - protein, 239 aa, Aequorea victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 6 - protein, 239 aa, Aequorea victoria
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 7 - DNA, 121 bp, Homo sapiens
  CDS (3)..(116)
ccatggccggtggtaccggttccggtgccctgaagaaggagctgcaggccaacaagaaggagctggcccagctgaagtgggagctgcaggccctgaagaaggagctggcccagtaggatcc

SEQ ID NO: 8 - protein, 37 aa, Homo sapiens
MAGGTGSGALKKELQANKKELAQLKWELQALKKELAQ

SEQ ID NO: 9 - DNA, 121 bp, Homo sapiens
  CDS (3)..(116)
ccatggccagcgagcagctggagaagaagctgcaggccctggagaagaagctggcccagctggagtggaagaaccaggccctggagaagaagctggcccagggcggcaccggttaggatcc

SEQ ID NO: 10 - protein, 37 aa, Homo sapiens
MASEQLEKKLQALEKKLAQLEWKNQALEKKLAQGGTG

SEQ ID NO: 11 - protein, 19 aa, Homo sapiens
GSGSGSGDITSLYKKAGST

SEQ ID NO: 12 - protein, 19 aa, Homo sapiens
GSGSGSGDITSLYKKAGST

SEQ ID NO: 13 - protein, 18 aa, Homo sapiens
DPAFLYKVVISGSGSGSG

SEQ ID NO: 14 - protein, 18 aa, Homo sapiens
DPAFLYKVVISGSGSGSG

SEQ ID NO: 15 - DNA, 23 bp, Artificial Sequence
  Synthetic oligonucleotide primer sequence
cctactgctttgagattcgtcgg

SEQ ID NO: 16 - DNA, 22 bp, Artificial Sequence
  Synthetic oligonucleotide primer sequence
gtcattccagttttagaagctc

SEQ ID NO: 17 - DNA, 28 bp, Artificial Sequence
  Synthetic oligonucleotide primer sequence
cagacaatctgtgtgggcactcgaccgg

SEQ ID NO: 18 - DNA, 47 bp, Artificial Sequence
  Synthetic oligonucleotide primer sequence
catggccggtggtaccggttccggtgccctgaagaaggagctgcagg

SEQ ID NO: 19 - DNA, 38 bp, Artificial Sequence
  Synthetic oligonucleotide primer sequence
agctccttcttcagggcaccggaaccggtaccaccggc

SEQ ID NO: 20 - DNA, 41 bp, Artificial Sequence
  Synthetic oligonucleotide primer sequence
ccaacaagaaggagctggcccagctgaagtgggagctgcag

SEQ ID NO: 21 - DNA, 40 bp, Artificial Sequence
  Synthetic oligonucleotide primer sequence
ctcccacttcagctgggccagctccttcttgttggcctgc

SEQ ID NO: 22 - DNA, 27 bp, Artificial Sequence
  Synthetic oligonucleotide primer sequence
gccctgaagaaggagctggcccagtag

SEQ ID NO: 23 - DNA, 37 bp, Artificial Sequence
  Synthetic oligonucleotide primer sequence
gatcctactgggccagctccttcttcagggcctgcag

SEQ ID NO: 24 - DNA, 40 bp, Artificial Sequence
  Synthetic oligonucleotide primer sequence
catggccagcgagcagctggagaagaagctgcaggccctg

SEQ ID NO: 25 - DNA, 31 bp, Artificial Sequence
  Synthetic oligonucleotide primer sequence
cctgcagcttcttctccagctgctcgctggc

SEQ ID NO: 26 - DNA, 45 bp, Artificial Sequence
  Synthetic oligonucleotide primer sequence
gagaagaagctggcccagctggagtggaagaaccaggccctggag

SEQ ID NO: 27 - DNA, 44 bp, Artificial Sequence
  Synthetic oligonucleotide primer sequence
ggcctggttcttccactccagctgggccagcttcttctccaggg

SEQ ID NO: 28 - DNA, 30 bp, Artificial Sequence
  Synthetic oligonucleotide primer sequence
aagaagctggcccagggcggcaccggttag

SEQ ID NO: 29 - DNA, 40 bp, Artificial Sequence
  Synthetic oligonucleotide primer sequence
gatcctaaccggtgccgccctgggccagcttcttctccag

SEQ ID NO: 30 - DNA, 24 bp, Artificial Sequence
  Synthetic oligonucleotide primer sequence
ggcgccatggtgagcaagggcgag

SEQ ID NO: 31 - DNA, 35 bp, Artificial Sequence
  Synthetic oligonucleotide primer sequence
gccggaccggtaccaccgttgtactccagcttgtg

SEQ ID NO: 32 - DNA, 33 bp, Artificial Sequence
  Synthetic oligonucleotide primer sequence
gccggaccggtaccaccctgcttgtcggccatg

SEQ ID NO: 33 - DNA, 36 bp, Artificial Sequence
  Synthetic oligonucleotide primer sequence
gccggaccggtaccaccctcgatgttgtggcggatc

SEQ ID NO: 34 - DNA, 32 bp, Artificial sequence
  Synthetic oligonucleotide primer sequence
ccccggatcctacttgtacagctcgtccatgc

SEQ ID NO: 35 - DNA, 36 bp, Artificial Sequence
  Synthetic oligonucleotide primer sequence
ggcgccatgggcaccggttacaacagccacaacgtc

SEQ ID NO: 36 - DNA, 36 bp, Artificial Sequence
  Synthetic oligonucleotide primer sequence
ggcgccatgggcaccggtaagaacggcatcaaggtg

SEQ ID NO: 37 - DNA, 36 bp, Artificial Sequence
  Synthetic oligonucleotide primer sequence
ggcgccatgggcaccggtgacggcagcgtgcagctc

SEQ ID NO: 38 - DNA, 34 bp, Artificial Sequence
  Synthetic oligonucleotide primer sequence
gggggctagcgccaccatggtgagcaagggcgag

SEQ ID NO: 39 - DNA, 57 bp, Artificial Sequence
  Synthetic oligonucleotide primer sequence
gcgggggatccgatatcgccagagccagagccagagccctcgatgttgtggcggatc

SEQ ID NO: 40 - DNA, 56 bp, Artificial Sequence
  Synthetic oligonucleotide primer sequence
gggggctagcgatatccggctctggctctggctctggcgacggcagcgtgcagctc

SEQ ID NO: 41 - DNA, 64 bp, Artificial Sequence
  Synthetic oligonucleotide primer sequence
gcccaccctcgtgaccaccttcggctacggcctgcagtgcttcgcccgctaccccgaccacatg

SEQ ID NO: 42 - DNA, 64 bp, Artificial Sequence
  Synthetic oligonucleotide primer sequence
catgtggtcggggtagcgggcgaagcactgcaggccgtagccgaaggtggtcacgagggtgggc

SEQ ID NO: 43 - DNA, 64 bp, Artificial Sequence
  Synthetic oligonucleotide primer sequence
gcccaccctcgtgaccaccctgggctacggcctgcagtgcttcgcccgctaccccgaccacatg

SEQ ID NO: 44 - DNA, 64 bp, Artificial Sequence
  Synthetic oligonucleotide primer sequence
catgtggtcggggtagcgggcgaagcactgcaggccgtagcccagggtggtcacgagggtgggc

SEQ ID NO: 45 - DNA, 36 bp, Artificial Sequence
  Synthetic oligonucleotide primer sequence
gacaaccactacctgagctaccagtccgccctgagc

SEQ ID NO: 46 - DNA, 36 bp, Artificial Sequence
  Synthetic oligonucleotide primer sequence
gctcagggcggactggtagctcaggtagtggttgtc

SEQ ID NO: 47 - DNA, 26 bp, Artificial Sequence
  Synthetic oligonucleotide primer sequence
ccaccatgggagtgcaggtggaaacc

SEQ ID NO: 48 - DNA, 19 bp, Artificial Sequence
  Synthetic oligonucleotide primer sequence
cttccagttttagaagctc

SEQ ID NO: 49 - DNA, 29 bp, Artificial Sequence
  Synthetic oligonucleotide primer sequence
ccaccatggagatgtggcatgaaggcctg

SEQ ID NO: 50 - DNA, 25 bp, Artificial Sequence
  Synthetic oligonucleotide primer sequence
cctgctttgagattcgtcggaacac

SEQ ID NO: 51 - DNA, 74 bp, Artificial Sequence
  Synthetic oligonucleotide primer sequence
tcctaggtcagtcctgctcctcggccacgaagtgcactcctaggctgcagcacgtgttgacaattaatcatcgg

SEQ ID NO: 52 - DNA, 28 bp, Artificial Sequence
  Synthetic oligonucleotide primer sequence
cagacaatctgtgtgggcactcgaccgg

SEQ ID NO: 53 - DNA, 59 bp, Artificial Sequence
  Synthetic oligonucleotide primer sequence
gcgggggatccgatatcgccagagccagagccagagccctgggccagctccttcttcag

SEQ ID NO: 54 - DNA, 28 bp, Artificial Sequence
  Synthetic oligonucleotide primer sequence
cctacttggagctggtgtacttggtgac

SEQ ID NO: 55 - DNA, 56 bp, Artificial sequence; identical to SEQ ID NO: 40
  Synthetic oligonucleotide primer sequence

SEQ ID NO: 56 - DNA, 29 bp, Artificial sequence
  Synthetic oligonucleotide primer sequence
ccaccatggctactcaagctgatttgatg

SEQ ID NO: 57 - DNA, 25 bp, Artificial Sequence
  Synthetic oligonucleotide primer sequence
ccaggtcagtatcaaaccaggccag

SEQ ID NO: 58 - DNA, 28 bp, Artificial Sequence
  Synthetic oligonucleotide primer sequence
cctacaggtcagtatcaaaccaggccag

SEQ ID NO: 59 - DNA, 36 bp, Artificial Sequence
  Synthetic oligonucleotide primer sequence
gttgctagcaccatgcctgagccagccaagtctgct

SEQ ID NO: 60 - DNA, 34 bp, Artificial Sequence
  Synthetic oligonucleotide primer sequence
gttgatatcccttggagctggtgtacttggtgac

SEQ ID NO: 61 - DNA, 29 bp, Artificial Sequence
  Synthetic oligonucleotide primer sequence
ccaccatgccgcagctgaacggcggtgga

SEQ ID NO: 62 - DNA, 22 bp, Artificial Sequence
  Synthetic oligonucleotide primer sequence
caggcggaggccgtctttccgc

SEQ ID NO: 63 - DNA, 36 bp, Artificial Sequence
  Synthetic oligonucleotide primer sequence
gttcagctgatgggagtgcaggtggaaaccatctcc

SEQ ID NO: 64 - DNA, 60 bp, Artificial Sequence
  Synthetic oligonucleotide primer sequence
gttggatccgatatcaccggtgcctgtcccagttccttccagttttagaagctccacatc

SEQ ID NO: 65 - DNA, 62 bp, Artificial Sequence
  Synthetic oligonucleotide primer sequence
gttgctagcgatatccggaacgggcactgggaccggtgagatgtggcatgaaggcctggaag

SEQ ID NO: 66 - DNA, 35 bp, Artificial Sequence
  Synthetic oligonucleotide primer sequence
gttcccgggagctgctttgagattcgtcggaacac

SEQ ID NO: 67 - DNA, 29 bp, Artificial Sequence
  Synthetic oligonucleotide primer sequence
ccaccatgcctgagccagccaagtctgct

SEQ ID NO: 68 - DNA, 25 bp, Artificial Sequence
  Synthetic oligonucleotide primer sequence
ccttggagctggtgtacttggtgac

SEQ ID NO: 69 - protein, 18 aa, Artificial Sequence
  Synthetic peptide linker sequence
DPAFLYKVVISGSGSGSG

SEQ ID NO: 70 - protein, 19 aa, Artificial sequence
  Synthetic peptide linker sequence
GSGSGSGDITSLYKKAGST

SEQ ID NO: 71 - protein, 16 aa, Artificial sequence
  Synthetic peptide linker sequence
DPAFLYKVVISGSGSG

SEQ ID NO: 72 - protein, 9 aa, Artificial sequence
  Synthetic peptide linker sequence
GSGSGSGDL

SEQ ID NO: 73 - protein, 18 aa, Artificial sequence
  Synthetic peptide linker sequence
DPAFLYKVVISGTGTGTG

SEQ ID NO: 74 - protein, 19 aa, Artificial sequence
  Synthetic peptide linker sequence
GSGSGSGDITSLYKKAGST

SEQ ID NO: 75 - protein, 12 aa, Artificial sequence
  Synthetic peptide linker sequence
DITSLYKKAGST

SEQ ID NO: 76 - protein, 19 aa, Artificial sequence
  Synthetic peptide linker sequence
GTGTGTGDITSLYKKAGST

SEQ ID NO: 77 - protein, 10 aa, Artificial sequence
  Synthetic peptide linker sequence
LPSGSGSGSG

SEQ ID NO: 78 - protein, 9 aa, Aequorea victoria
LTYGVQCFS

SEQ ID NO: 79 - protein, 9 aa, Artificial Sequence
  synthetically mutated peptide L64FT65GV68L S72A
FGYGLQCFA

SEQ ID NO: 80 - protein, 9 aa, Artificial Sequence
  synthetically mutated peptide T65GV68LS72A
LGYGLQCFA

SEQ ID NO: 81 - protein, 7 aa, Artificial Sequence
  Synthetic peptide linker sequence
GSGSGSG

SEQ ID NO: 82 - protein, 6 aa, Artificial Sequence
  Synthetic peptide linker sequence
GGTGSG

SEQ ID NO: 83 - protein, 7 aa, Artificial Sequence
  Synthetic peptide linker sequence
GTGTGTG

SEQ ID NO: 84 - protein, 238 aa, Aequorea Victoria; identical to SEQ ID NO: 1

SEQ ID NO: 85 - protein, 238 aa, Renilla muelleri
MSKQILKNTCLQEVMSYKVNLEGIVNNHVFTMEGCGKGNILFGNQLVQIRVTKGAPLPFAFDIVSPAFQYGNRTFTKYPNDISDYFIQSFPAGFMYERTLRYEDGGLVEIRSDINLIEDKFVYRVEYKGSNFPDDGPVMQKTILGIEPSFEAMYMNNGVLVGEVILVYKLNSGKYYSCHMKTLMKSKGVVKEFPSYHFIQHRLEKTYVEDGGFVEQHETAIAQMTSIGKPLGSLHEWV

SEQ ID NO: 86 - protein, 233 aa, Renilla reniformis
MDLAKLGLKEVMPTKINLEGLVGDHAFSMEGVGEGNILEGTQEVKISVTKGAPLPFAFDIVSVAFSYGNRAYTGYPEEISDYFLQSFPEGFTYERNIRYQDGGTAIVKSDISLEDGKFIVNVDFKAKDLRRMGPVMQQDIVGMQPSYESMYTNVTSVIGECIIAFKLQTGKHFTYHMRTVYKSKKPVETMPLYHFIQHRLVKTNVDTASGYVVQHETAIAAHSTIKKIEGSLP

SEQ ID NO: 87 - protein, 225 aa, Discosoma sp.
MRSSKNVIKEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERTEGRHHLFL

SEQ ID NO: 88 - protein, 148 aa, Anemonia sulcata
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPRDAEQSRKMGASHRDTL

SEQ ID NO: 89 - protein, 231 aa, Zoanthus sp.
MAQSKHGLTKEMTMKYRMEGCVDGHKFVITGEGIGYPFKGKQAINLCVVEGGPLPFAEDILSAAFNYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 90 - protein, 232 aa, Anemonia sulcata
MASFLKKTMPFKTTIEGTVNGHYFKCTGKGEGNPFEGTQEMKIEVIEGGPLPFAFHILSTSCMYGSKTFIKYVSGIPDYFKQSFPEGFTWERTTTYEDGGFLTAHQDTSLDGDCLVYKVKILGNNFPADGPVMQNKAGRWEPATEIVYEVDGVLRGQSLMALKCPGGRHLTCHLHTTYRSKKPASALKMPGFHFEDHRIEIMEEVEKGKCYKQYEAAVGRYCDAAPSKLGHN

SEQ ID NO: 91 - protein, 228 aa, Anemonia sulcata
MYPSIKETMRVQLSMEGSVNYHAFKCTGKGEGKPYEGTQSLNITITEGGPLPFAFDILSHAFQYGIKVFAKYPKEIPDFFKQSLPGGFSWERVSTYEDGGVLSATQETSLQGDCIICKVKVLGTNFPANGPVMQKKTCGWEPSTETVIPRDGGLLLRDTPALMLADGGHLSCFMETTYKSKKEVKLPELHFHHLRMEKLNISDDWKTVEQHESVVASYSQVPSKLGHN

SEQ ID NO: 92 - protein, 225 aa, Montastraea cavernosa
MSVIKPIMEIKLRMQGVVNGHKFVIKGEGEGKPFEGTQTINLTVKEGAPLPFAYDILTSAFQYGNRVFTKYPDDIPDYFKQTFPEGYSWERIMAYEDQSICTATSDIKMEGDCFIYEIQFHGVNFPPNGPVMQKKTLKWEPSTEKMYVRDGVLKGDVNMALLLEGGGHYRCDFRSTYKAKKRVQLPDYHFVDHRIEILSHDNDYNTVKLSEDAEARYSMLPSQAK

SEQ ID NO: 93 - protein, 227 aa, Montastraea faveolata
MSVIKPDMKIKLRMEGAVNGHKFVIEGDGKGKPFEGTQSMDLTVKEGAPLPFAYDILTTVFDYGNRVFAKYPQDIPDYFKQTFPEGYSWERSMTYEDQGICVATNDITLMKGVDDCFVYKIRFDGVNFPANGPVMQKKTLKWEPSTEKMYVRDGVLKGDVNMALLLEGGGHYRCDFKTTYKAKKFVQLPDYHFVDHRIEILSHDKDYNKVKLYEHAEAHSGLPRQAK

SEQ ID NO: 94 - protein, 266 aa, Clavularia sp.
MKCKFVFCLSFLVLAITNANIFLRNEADLEEKTLRIPKALTTMGVIKPDMKIKLKMEGNVNGHAFVIEGEGEGKPYDGTHTLNLEVKEGAPLPFSYDILSNAFQYGNRALTKYPDDIADYFKQSFPEGYSWERTMTFEDKGIVKVKSDISMEEDSFIYEIRFDGMNFPPNGPVMQKKTLKWEPSTEIMYVRDGVLVGDISHSLLLEGGGHYRCDFKSIYKAKKVVKLPDYHFVDHRIEILNHDKDYNKVTLYENAVARYSLLPSQA

SEQ ID NO: 95 - protein, 232 aa, Discosoma striata
MSCSKSVIKEEMLIDLHLEGTFNGHYFEIKGKGKGQPNEGTNTVTLEVTKGGPLPFGWHILCPQFQYGNKAFVHHPDNIHDYLKLSFPEGYTWERSMHFEDGGLCCITNDISLTGNCFYYDIKFTGLNFPPNGPVVQKKTTGWEPSTERLYPRDGVLIGDIHHALTVEGGGHYACDIKTVYRAKKAALKMPGYHYVDTKLVIWNNDKEFMKVEEHEIAVARHHPFYEPKKDK

SEQ ID NO: 96 - protein, 238 aa, Ptilosarcus sp.
MNRNVLKNTGLKEIMSAKASVEGIVNNHVFSMEGFGKGNVLFGNQLMQIRVTKGGPLPFAFDIVSIAFQYGNRTFTKYPDDIADYFVQSFPAGFFYERNLRFEDGAIVDIRSDISLEDDKFHYKVEYRGNGFPSNGPVMQKAILGMEPSFEVVYMNSGVLVGEVDLVYKLESGNYYSCHMKTFYRSKGGVKEFPEYHFIHHRLEKTYVEEGSFVEQHETAIAQLTTIGKPLGSLHEWV

SEQ ID NO: 97 - protein, 231 aa, Zoanthus sp.
MAHSKHGLKEEMTMKYHMEGCVNGHKFVITGEGIGYPFKGKQTINLCVIEGGPLPFSEDILSAGFKYGDRIFTEYPQDIVDYFKNSCPAGYTWGRSFLFEDGAVCICNVDITVSVKENCIYHKSIFNGMNFPADGPVMKKMTTNWEASCEKIMPVPKQGILKGDVSMYLLLKDGGRYRCQFDTVYKAKSVPSKMPEWHFIQHKLLREDRSDAKNQKWQLTEHAIAFPSALA

SEQ ID NO: 98 - protein, 229 aa, Anemonia majano
MALSNKFIGDDMKMTYHMDGCVNGHYFTVKGEGNGKPYEGTQTSTFKVTMANGGPLAFSFDILSTVFKYGNRCFTAYPTSMPDYFKQAFPDGMSYERTFTYEDGGVATASWEISLKGNCFEHKSTFHGVNFPADGPVMAKKTTGWDPSFEKMTVCDGILKGDVTAFLMLQGGGNYRCQFHTSYKTKKPVTMPPNHVVEHRIARTDLDKGGNSVQLTEHAVAHITSVVPF

SEQ ID NO: 99 - protein, 238 aa, Aequorea macrodactyla
MSKGEELFTGIVPVLIELDGDVHGHKFSVRGEGEGDADYGKLEIKFICTTGKLPVPWPTLVTTFSYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSTQTAISKDRNETRDHMVFLEFFSACGHTHGMDELYK

(19) United States
(12) Patent Application Publication
(10) Pub. No.: US 2006/0247419 A1
(43) Pub. Date: Nov. 2, 2006

(54) Translocation dependent complementation for drug screening

(75) Inventors: Søren Weis Dahl, Rungsted (DK); Bernard Robert Terry, Frederiksberg (DK); Morten Praestegaard, Ballerup (DK); Soren Jensby, Nielsen (DK); Grith Hagel, Dragor (DK)

Correspondence Address: BIRCH STEWART KOLASCH & BIRCH, PO BOX 747, FALLS CHURCH, VA, 22040-0747, US

(73) Assignee: BioImage A/S, Soborg (DK)

(21) Appl. No.: 10/511,468

(22) PCT Filed: Apr. 22, 2003
(86) PCT No.: PCT/DK03/00266

§ 371 Date: Oct. 18, 2004

(30) Foreign Application Priority Data
Apr. 19, 2002 (DK) PA 2002 00591
Jun. 13, 2002 (DK) PA 2002 00896
Jul. 1, 2002 (DK) PA 2002 01029
Dec. 19, 2002 (DK) DK02/00882

(51) Int. Cl. C07K 14/705; G01N 33/53; C07H 21/04; C12P 21/06; C12N 1/21

(52) U.S. Cl. 530/350; 435/7.1; 435/69.1; 435/320.1; 435/325; 435/252.3; 536/23.5

(57) ABSTRACT

The present invention relates to various uses of complementing proteins protein fragments to discover chemical compounds or drugs interfering with protein translocation/redistribution and/or protein interactions. The invention takes advantage of the fact that many interacting proteins reside in separate and distinct locations prior to the activation of the signaling pathways in which they play their part. The invention also takes advantage of interaction domains (FRBP, FKBP12) that can be induced to interact. Specifically disclosed are GFP (EGFP, F64L mutated EYFP) complementation.

### FIELD OF INVENTION

The present invention relates to various uses of complementing proteins protein fragments to discover chemical compounds or drugs interfering with protein translocation and/or protein-protein interactions. The invention takes advantage of the fact that many interacting proteins reside in separate and distinct locations prior to the activation of the signaling pathways in which they play their part. The invention also takes advantage of certain interaction domains that can be induced to interact by application of specific stimuli, by which means the complementation signal may be enhanced for those complementary components that become directed to the same intracellular location following activation of the signaling pathway in which they act.

### BACKGROUND OF THE INVENTION

The interaction of proteins with each other and with other cellular components is an intrinsic part of nearly every cellular process, and this is especially true of intracellular signaling systems. Information is typically passed through and between signaling systems by a series of such interactions.

Existing methods for identification of interacting species can be divided into two groups: First are those methods that can only work with more or less purified components brought together in vitro, such as surface plasmon resonance (evanescent wave methods), protein mass spectroscopy, fluorescence correlation spectroscopy and anisotropy measurements all with the common feature that the components of interest are isolated from the cellular context. The second group includes all methods designed to work within living cells. Of these, many have been developed to work in yeast cells (yeast two hybrid, reverse yeast two hybrid and variations thereof) but some have been adapted for use in mammalian cell systems. Cellular methods for detection of protein Interactions have been well reviewed by Mendelsohn, A. R., Brent, R. (1999) (Science 284(5422):1948). Many of these methods are descendants of the conventional two-hybrid methods, where transcriptional activity is initiated by the bringing together of bi-partite transcription factors through the interaction of attached “bait” and “prey” components, while other methods rely on reconstitution of a biochemical function in vivo. Rossi et al. (2000) (Trends in Cell Biology 10:119-122) have thus developed a mammalian cell-based protein-protein interaction assay where the read-out is not transcriptional but reconstitution of a mutated beta-galactosidase enzyme. Upon reconstitution of the tetrameric enzyme, enzymatic activity can be monitored. In addition methods for monitoring protein-protein interactions that are based on an optical read-out i.e. fluorescence resonance transfer (FRET), or coincidence analysis (a variant of fluorescence correlation spectroscopy), or fluorescence lifetime changes. The last three categories are more normally applied under simplified in vitro conditions, but attempts are being made to move them into the more complex environment of the living cell.

It has been suggested to use the reassembly of certain enzyme fragments of the complete enzyme as a measure of protein-protein interactions. Johnsson and Varshavsky (Johnsson, N., Varshavsky, A. (1994) Proc. Natl. Acad. Sci. U.S.A. 91, 10340-10344) disclose reassembly of Ubiquitin. This reassembly is detected through the irreversible cleavage of the fusion by Ubiquitin protease and release of a reporter. As opposed to the two-hybrid technique, this technique includes the possibility of monitoring a protein-protein interaction as a function of time, at the natural sites of this interaction in a living cell.

Similar systems are suggested for the reassembly of other proteins including β-galactosidase (Rossi, F., Charlton, C. A., Blau, H. M. (1997) Proc. Natl. Acad. Sci. U.S.A. 94, 8405-8410), dihydrofolate reductase (DHFR, WO98/34120), and β-lactamase (Wehrman, T., Kleaveland, B., Her, J. H., Balint, R. F., Blau, H. M. (2002) Proc. Natl. Acad. Sci. U.S.A. 99, 3469-3474). The basic concept is that by splitting a functional protein in two fragments, the function is lost. The two fragments are transformed or transfected into cells fused in frame to proteins X and Y, respectively. Binding between proteins X and Y will bring the two fragments close together, increasing the local concentration of the complementing fragments, induce folding of these fragments and produce a functional protein with an activity that is similar to that of the non-fragmented protein. If the function is DHFR activity, the cells will survive only if proteins X and Y bind to each other.

Recently, it has been described to use a somewhat similar system for the assisted reassembly and folding of fragments of fluorescent proteins. As the function is fluorescence, the cells will emit light upon excitation only if protein X and protein Y bind to each other thereby assisting complementation. Ghosh (I. Ghosh, A. D. Hamilton, L. Regan (2000) J. Am. Chem. Soc. 122, 5658-5659, WO01/87919) describes the use of a GFP variant called sg100 (F64L, S65C, Q80R, Y151 L, I167T and K238N). This GFP has single fluorescence excitation and emission peaks at 475 nm and 505 nm, respectively (similar to sg25 described by Palm (Palm, G. J., Zdanov, A., Gaitanaris, G. A., Stauber, R., Pavlakis, G. N., Wlodawer, A. (1997) Nat. Struct. Biol. 4, 361-365)). Functional GFP fragment complementation is accomplished by co-expressing two independent peptides composed of the first 157 N-terminal amino acids of this GFP (NtermGFP157) and the remaining 81 C-terminal amino acids (starting form residue 158) of this GFP (CtermGFP158) with each of the GFP peptide fragments being fused to interacting leucine zipper peptides that serve to associate the fragments.

The recruitment or re-localisation of proteins plays a major role in many key signalling systems. This is evident in (i) the activation of protein kinase C (PKC), where recruitment to the plasma membrane is an inherent part of the activation process of this enzyme; (ii) the activation of p42/44 MAP Kinase depends on multiple proteins whose transfer from cytosolic to membrane location has a central role and (iii) the cAMP-driven relocalisation of rap1 is crucial to its activation. The use of protein translocation for identification of potential medicaments is disclosed in WO98/45704. However, there is still a need for alternative techniques for detecting the localisation and any re-localisation of proteins in intact, living cells. Current detection techniques are generally characterised by low throughputs and requirements for expensive detection equipments.

### SUMMARY OF THE INVENTION

The present application discloses that certain GFPs can be reassembled and form a functional fluorescent protein when expressed as two independent proteins halves. For example, when EGFP is expressed in mammalian cells, choosing a split site located in a loop region between the residues that form the beta-sheet structures of the GFP beta-barrel results in intense fluorescence (Example 5 and Example 7). The present application further illustrates that EYFP is also reassembled and, surprisingly, the fluorescence from the reassembled protein is markedly enhanced if it contains the F64L mutation (Example 9).

The present application demonstrates that a model GFP complementation system using components which can be made to interact conditionally does respond as expected in a dose-dependent manner to the interaction stimulus, and, importantly, can be used to detect compounds that will block the conditional interaction of proteins fused to the complementary halves of a protein (e.g. a GFP complementation system) (Example 11) as almost no spontaneous reassembly (complementation) is observed.

The examples herein disclose that for spontaneous interacting proteins, a complementation system can be designed to report on the degree of interaction between those proteins in living cells, with the option to add compounds before such interaction takes place. This is achieved by holding the two proteins in separate locations of the cell thereby only allowing interaction to take place after translocation of one or both proteins to bring them together in one compartment or location within the cell has taken place (Example 16).

One aspect of the present invention builds on the concept that signaling proteins which will interact with each other when active often reside in distinct and spatially separated intracellular locations prior to the activation of the signaling pathway in which they act, thus avoiding spontaneous reassembly of the complementary system used to monitor the interaction of those proteins. This is clearly illustrated in Example 14, where induction of translocation from one localisation to another causes the N- and C-terminal of the complementation proteins to reassemble and exert the function (in this case fluorescence). A similar system is described in Example 16 where constitutive interacting proteins has been replaced with proteins of induced interaction.

### DETAILED DISCLOSURE

The present invention builds on the knowledge of localisation of proteins inside the cell and using this to keep the two half's of an enzyme apart until in a controlled fashion they are brought back together. Thus, a major embodiment of the invention relates to a cell comprising at least:
a first conjugate comprising a first protein and the N-terminal fragment of complementation protein; and
a second conjugate comprising a second protein and the C-terminal fragment of the complementation protein;
   wherein said first conjugate has a predominant cellular location that is distinct and spatially separated from where said second conjugate is predominantly located,
   wherein said first protein and said second protein will bind to each other when conditions allow, and
   wherein said complementation protein exhibits altered characteristics when the two fragments of the complementation protein are brought into close apposition and the two fragments of the complementation protein form the full functional protein.

One use of the invention is to be able to identify drugs that will prevent the interaction between the first protein and the second protein. To avoid interaction before such drug has been applied, one important feature of the invention is that the two conjugates are held in different cellular locations. Most, if not all, proteins are in equilibrium between various states. In the present context, this equilibrium will represent the balance between being in one location and being in another location within the cell. For the purpose of the present application, two different cellular locations are understood as predominant or preferred positions of the two proteins making interaction between the two proteins unlikely, that is, the conditions for their meeting are not favored. In the cases where the balance between two locations is shifted far to one side, the protein will predominantly be in one of these locations. Activation of the system here means the result of any treatment of the system that initiates translocation, or any other cellular signaling process, that makes complementation of the two conjugates more favorable—that is, activation of the system shifts the equilibrium of at least one of the proteins from a different cellular location from the other protein, to the same cellular location as the other protein. As an example, PKCβ proteins are found predominantly, but not exclusively, in the cytoplasm in unstimulated cells, whereas the interaction partner protein, RACK1, is predominantly located at the plasma membrane. Upon application of a stimulus such as the phorbol ester PMA, PKCβ proteins take up a location predominantly at the plasma membrane, where they are therefore brought into close proximity to RACK1. Another example of signaling proteins that translocate to and from the plasma membrane are STAT transcription factors and AKT/PBK kinase. Examples of signaling proteins that translocate between the cytoplasm and the nucleus are NF kappaB transcription factor, the androgen receptor, the MAP kinases p38, ERK and JNK, MAPKAP kinase 2 and Forkhead transcription factor.

Thus for most uses of the invention, the cellular location of the first conjugate is determined by the nature of the first protein. Likewise, the cellular location of the second conjugate is determined by the nature of the second protein.

The presently known complementation proteins causes irreversible complementation because the correctly folded complexes of the complementing fragments are energetically very favourable, i.e. the folded fragments dissociate only very slowly if at all. Thus, if the complementation starts as soon as the proteins are in the same location, due to spontaneous binding between the first protein in the first conjugate and the second protein in the second conjugate, the small fraction of protein in the not favored location will be irreversibly bound. When irreversibly bound, it no longer participates in the equilibrium of free protein conjugate between the unfavored and favored locations and inevitably more of the protein conjugate can go to the unfavored location. Ultimately, most or all of the protein conjugate can be drawn by the equilibrium to the unfavored location. This is referred to a sink effect. One way to minimize the spontaneous meeting between the two proteins, is to associate one of the proteins with an anchor protein that will severely limit its mobility in the cell. In this case the one protein is fixed in one localisation, and the meeting between the complementing fragments will only take place if the other protein is moving to the anchored position.

In this aspect of the invention, the second conjugate further comprises an anchor protein, wherein said anchor protein is anchored in a different cellular compartment from where said first protein is predominantly located.

Another use of the location information wherein constitutively interacting proteins are kept apart until the two conjugates are purposely brought together is to measure translocation of a protein. This can be done in a cell comprising at least:
a first conjugate comprising a protein, interaction partner A and the first terminal fragment of a complementation protein; and
a second conjugate comprising a interaction partner B and the second terminal fragment of the complementation protein;
   wherein said first conjugate has a predominant cellular location that is distinct and spatially separated from where said second conjugate is predominantly located,
   wherein said complementation protein exhibits altered characteristics when the two fragments of the complementation protein are brought into close apposition and the two fragments of the complementation protein form the full functional protein and
   wherein said interaction partner A and interaction partner B bind to each other.

In such cell, translocation of the protein, and thereby the first conjugate, to the same cellular location as the second conjugate, will cause complementation of the first and second terminal fragments of the complementation protein.

One way to minimize the effect of spontaneous complementation between the two proteins (the sink effect, which generates an unwanted background signal), is to include in the conjugates interaction partners A and B that will only bind when a specific interaction stimulus has been applied (conditional interaction partners). In this way the spontaneous meeting between the proteins will only cause complementation of the two complementation fragments when the interaction stimulus causes the interaction partners to bind and thereby bring the complementation fragments in close apposition for the reassembly and formation of a functional protein. In this embodiment interaction partner A and interaction partner B bind to each other only when an interaction stimulus has been applied.

To obtain further control of the location of the conjugates and minimize the spontaneous meeting between the two conjugates, is to associate one of the conjugates with an anchor protein that will severely limit its mobility in the cell. In this case the one protein is fixed in one localisation, and the meeting between the complementing fragments will only take place if the other protein is moving to the anchored position. In this aspect of the invention, the second conjugate further comprises an anchor protein, wherein said anchor protein is anchored in a different cellular compartment from where said first protein is predominantly located.

In one embodiment of the invention the first terminal fragment of the complementation protein is the N-terminal fragment of the complementation protein and the second terminal fragment of the complementation protein is the C-terminal fragment of the complementation protein. In another embodiment of the invention the first terminal fragment of the complementation protein is the C-terminal fragment of the complementation protein and the second terminal fragment of the complementation protein is the N-terminal fragment of the complementation protein.

The simplicity of the present invention is in part derived from the complementation protein. The fact that the complementation protein exhibits altered characteristics when the two fragments of the complementation protein are brought into close apposition and the two fragments of the complementation protein form the full functional protein, makes detection of the close apposition between the two fragments of the complementation protein easy. In principle any detectable property of a combined mass that is measurably different to that of the separate constituent parts may be used as a signal of complementation of those separate parts. Such properties include, but are not limited to, biophysical properties such as rotational or translational diffusion coefficients, fluorescence properties such as peak excitation or emission wavelengths, or fluorescence lifetimes. Potentially useful detectable properties also include those of a more biochemical nature, such as the acquisition of catalytic efficiency or enzymic activity, or the efficacy of conversion of a polypeptide sequence inherent to one of the complementary partners to a form that acquires the ability to fluoresce or luminesce. Examples of proteins that can be made to acquire catalytic activity when the constituent parts are assembled through complementation are Ubiquitin (Johnsson, N., Varshavsky, A. (1994)) β-galactosidase (Rossi, F et al (1997)) and β-lactamase (Wehrman, et al (2002)).

Below it is described in detail how GFP can be divided to form two complementing fragments, as such data are not available on the priority date of the present invention. However, as will be evident to the skilled person, other proteins can be divided as well. The non-fluorescent fragments of fluorescent proteins that can be combined to form one functional fluorescent unit are usually produced by splitting the coding nucleotide sequence of one fluorescent protein at an appropriate site and expressing each nucleotide sequence fragment independently (as fusion proteins) as the N-terminal fragment and the C-terminal fragment of the complementation protein (here GFP).

In one embodiment of the invention, the complementation protein is a fluorescent protein such as a Green Fluorescent Protein (GFP).

Green Fluorescent Protein (GFP) is a 238 amino acid long protein derived from the jellyfish Aequorea Victoria (SEQ ID NO: 1). However, fluorescent proteins have also been isolated from other members of the Coelenterata, such as the red fluorescent protein from Discosoma sp. (Matz, M. V. et al. 1999, Nature Biotechnology 17: 969-973), GFP from Renilla reniformis, GFP from Renilla Muelleri or fluorescent proteins from other animals, fungi or plants. The GFP exists in various modified forms including the blue fluorescent variant of GFP (BFP) disclosed by Heim et al. (Heim, R. et al., 1994, Proc. Natl. Acad. Sci. 91:26, pp 12501-12504) which is a Y66H variant of wild type GFP; the yellow fluorescent variant of GFP (YFP) with the S65G, S72A, and T203Y mutations (WO98/06737); the cyan fluorescent variant of GFP (CFP) with the Y66W colour mutation and optionally the F64L, S65T, N146I, M153T, V163A folding/solubility mutations (Heim, R., Tsien, R. Y. (1996) Curr. Biol. 6,178-182). The most widely used variant of GFP is EGFP with the F64L and S65T mutations (WO 97/11094 and WO96/23810) and insertion of one valine residue after the first Met. The F64L mutation is the amino acid in position 1 upstream from the chromophore. GFP containing this folding mutation provides an increase in fluorescence intensity when the GFP is expressed in cells at a temperature above about 30° C. (WO 97/11094).

It is known that fluorescence in wild-type GFP is due to the presence of a chromophore, which is generated by cyclisation and oxidation of the SYG at position 65-67 in the predicted primary amino acid sequence and presumably by the same reasoning of the SHG sequence in other GFP analogues at positions 65-67.

The present examples clearly illustrate how the fluorescence intensity from a reassembled protein is enhanced in GFPs containing the F64L mutations as compared to GFPs without this mutation. Thus, it is preferred that the GFP contains the F64L mutation, either by electing a GFP with this mutation (e.g. EGFP) or to introduce this mutation into the GFP of choice (e.g. YFP as illustrated in Example 9).

In the nomenclature of GFP, an “E” is placed in front of the GFP (EGFP, EYFP, ECFP) to indicate that this particular GFP is encoded by a nucleic acid with codon usage optimised for mammalian cells. Most of these proteins also have an extra valine residue inserted after the initial methionine residue, Met¹. This extra valine residue is not considered in the numbering of the residues. Thus, in a preferred embodiment, the GFP of the present invention is selected from the group consisting of EGFP, EYFP, ECFP, dsRed and Renilla GFP.

Some of the examples of the present application, EGFP is used. Thus, in a preferred embodiment of the invention, the GFP is EGFP. However, Example 9 and Example 11 show that EYFP has certain advantages. Thus, in another preferred embodiment of the invention, the GFP is EYFP. It is also shown that EYFP mutated in position 1 preceding the chromophore (E[F64L]YFP) has specific advantages. Thus, in a preferred embodiment the GFP is E[F64L]YFP, or the GFP is E[S72A]GFP (that is F64L, S65T, S72A GFP).

One aspect of the invention relates to a cell as described above, wherein N-terminal fragment of the complementation protein is an N-terminal fragment of GFP, comprising a continuous stretch of amino acids from amino acid number 1 to amino acid number X of GFP, wherein the peptide bond between amino acid number X and amino acid number X+1 is within a loop of GFP and C-terminal fragment of the complementation protein is a C-terminal fragment of GFP, comprising a continuous stretch of amino acids from amino acid number X+1 to amino acid number 238 of GFP.

In the present context, the numbering of wild-type GFP (SEQ ID NO: 1) (Chalfie, M., Tu, Y., Euskirchen, G., Ward, W. W., Prasher, D. C. (1994) Science 263, 802-805, this variant of GFP has a histidine residue in position 231) is used. Based on the crystal structure of GFP (Yang, F., Moss, L. G., Phillips, G. N. (1996) Nat. Biotech. 14, 1246-1251) FIG. 5, Table 1 and the data presented in the examples, it is evident that a split in almost any loop will be re-assembled following appropriate spatial approximation to the complementation fragments assisted by the interaction of the conjugated proteins. For the purpose of this application the term “loop” shall be understood as a turn or element of irregular secondary structure.

Thus, in one aspect, the complementation protein is two GFP fragments
wherein X is 7, 8, 11 or 12, preferably X is 9 or 10 within the Thr9-Val11 loop; or
wherein X is 21, 22, 25 or 26, preferably X is 23 or 24 within the Asn23-His25 loop; or
wherein X is 36, 37, 40 or 41, preferably X is 38 or 39 within the Thr38-Gly40 loop; or
wherein X is 46, 47, 56 or 57, preferably X is between 48 and 55 i.e. X is 48, 49, 50, 51, 52, 53, 54 or 55 within the Cys48-Pro56 loop; or
wherein X is 70, 71, 76 or 77, preferably X is between 72 and 75 i.e. X is 72, 73, 74 or 75 within the Ser72-Asp76 loop; or
wherein X is 79, 80, 83 or 84, preferably X is 81 or 82 within the His81-Phe83 loop; or
wherein X is 86, 87, 90 or 91, preferably X is 88 or 89 within the Met88-Glu90 loop; or
wherein X is 99, 100, 103 or 104, preferably X is 101 or 102 within the Lys101-Asp 03 loop; or
wherein X is 112, 113, 118 or 119, preferably X is between 114 and 117 i.e. X is 114, 115, 116 or 117 within the Phe114-Thr118 loop; or
wherein X is 126, 127, 145 or 146, preferably X is between 128 and 144 i.e. X is 128, 129, 130, 131, 132, 133, 134, 135, 136, 137, 138, 139, 140, 141, 142, 143, 144 within the Ile 128-Tyr145 loop; or
wherein X is 152, 153, 160 or 161, preferably X is between 154 and 159 i.e. X is 154, 155, 156, 157, 158 or 159 within the Ala154-Gly160 loop; or
wherein X is 169, 170, 175, 176, preferably X is between 171 and 174 i.e. X is 171, 172, 173 or 174 within the Ile171-Ser175 loop; or
wherein X is 186, 187, 197 or 198, preferably X is between 188 and 196 i.e. X is 188, 189, 190, 191, 192, 193, 194, 195 or 196 within the Ile188-Asp197 loop; or

wherein X is 208, 209, 215 or 216, preferably X is between 210 and 214 i.e. X is 210, 211, 212, 213 or 214 within the Asp210-Art215 loop.

TABLE 1
GFP secondary structures, GFP wild type sequence amino
acid numbering, α and β indicate α-helical
and β-sheet secondary structures, respectively.

| Name | Position | |
|---|---|---|
| Helix 1 | Lys3-Thr9 | α1 |
| Sheet 1 | Val11-Asn23 | β1 |
| Sheet 2 | His25-Thr38 | β2 |
| Sheet 3 | Gly40-Cys48 | β3 |
| Helix 2 | Pro56-Ser72 | α2 |
| Helix 3 | Asp76-His81 | α3 |
| Helix 4 | Phe83-Met88 | α4 |
| Sheet 4 | Glu90-Lys 101 | β4 |
| Sheet 5 | Asp103-Phe114 | β5 |
| Sheet 6 | Thr118-Ile128 | β6 |
| Sheet 7 | Tyr145-Ala154 | β7 |
| Sheet 8 | Gly160-Ile171 | β8 |
| Sheet 9 | Ser175-Ile188 | β9 |
| Sheet 10 | Asp197-Asp210 | β10 |
| Sheet 11 | Arg215-Gly228 | β11 |

Based on the findings disclosed in the examples, it is concluded that appropriate splitting sites in GFP are located in the loop regions between the residues that form the beta-sheet structures of the GFP beta-barrel. Accordingly, splits in GFP are preferably made in the Asn23-His25 loop, the Thr38-Gly40 loop, the Lys101-Asp102 loop, the Phe114-Thr118 loop, the Ile128-Tyr145 loop, the Ala154-Gly160 loop, the Ile171-Ser175 loop, the Ile188-Asp197 loop or the Asp210-Arg215 loop (Table 1, FIG. 5).

The data in the present examples illustrates clearly that the Ala154-Gly160 loop is very well suited for GFP reassembly. This is particularly the case when the GFP is divided between amino acids Q157 and K158 (that is, when X is 157). Thus, a preferred embodiment of the invention relates to two GFP fragments, wherein X is 157 within the Ala154-Gly160 loop.

The data in the present examples also illustrate that the Ile171-Ser175 loop is very useful for GFP reassembly. This is particularly the case, when the GFP is divided between amino acids E172 and D173 (that is, when X is 172). Thus, a preferred embodiment of the invention relates to two GFP fragments, wherein X is 172 within the Ile171-Ser175 loop.

One alterative aspect of the invention relates to the cell described above, wherein the N-terminal fragment of the complementation protein is an N-terminal fragment of GFP, comprising a continuous stretch of amino acids from amino acid number 1 to amino acid number X of GFP, wherein the peptide bond between amino acid number X and amino acid number X+1 is within a loop of GFP and the C-terminal of the complementation protein is a C-terminal fragment of GFP, comprising a continuous stretch of amino acids from amino acid number Y+1 to amino acid number 238 of GFP, wherein Y<X creating an overlap of the two GFP fragments, and wherein the peptide bond between amino acid Y and amino acid Y+1 is within a loop of GFP.

As illustrated in Example 9, fragments having overlapping sequences have certain advantages. Thus one aspect of the invention relates to two GFP fragments comprising

(a) an N-terminal fragment of GFP, comprising a continuous stretch of amino acids from amino acid number 1 to amino acid number X of GFP, wherein the peptide bond between amino acid number X and amino acid number X+1 is within a loop of GFP and

(b) a C-terminal fragment of GFP, comprising a continuous stretch of amino acids from amino acid number Y+1 to amino acid number 238 of GFP, wherein Y<X creating an overlap of the two GFP fragments, and wherein the peptide bond between amino acid Y and amino acid Y+1 is within a loop of GFP.

These overlapping GFP fragments are very attractive in e.g. functional cloning systems where highly flexible linkers sequences are required due to the very diverse nature of the structures of fusion partners. The overlapping fragments permit either of the fusion partners to have a long linker sequence.

For the purposes of deciding the nature of the Y in the C-terminal fragment of GFP defined above, the same considerations as discussed for the value of X applies.

In one embodiment of the invention the overlap is just a few amino acid residues, e.g. X-Y=1, X-Y=2, X-Y=3, X-Y=4, X-Y=5, X-Y=6, X-Y=7, X-Y=8, X-Y=9 or X-Y=10.

Due to the folding characteristics of the folding of GFP, a preferred embodiment of the invention relates to overlapping N-terminal and C-terminal fragments of GFP wherein the peptide bond between amino acid Y and amino acid Y+1 and the peptide bond between amino acid X and amino acid X+1 is within a loop of GFP. The thereby obtained overlap is an entire α-helix or β-sheet secondary structure

In a typical embodiment of the invention, the conjugated protein as described above, wherein the N-terminal fragment of the complementation protein (the first terminal fragment of the complementation protein) is conjugated to a protein of interest, further comprises a linker sequence between the fragment of the complementation protein and the corresponding protein of interest.

The length and composition of the linker must be chosen depending on the protein that one want conjugated to a the complementation protein fragment. The main function of a linker is to provide flexibility to avoid steric hindrance of the complementation. The interacting proteins should be free to interact and at the same time the complementing fragments should be free to fold. This is facilitated by long linker sequences, e.g. linkers of 20 or more residues. However short linker sequences will keep the complementation protein fragments closer to each other, i.e. increase the local concentration of complementing fragments, and decrease the entropy cost of complementation. Access to information about the structures of the interacting proteins or homologous proteins and to information about the interaction between the interacting proteins can be valuable because it may facilitate the design of short linkers of 5-10 residues that do not distort the protein interaction or complementation. It will often be advantageous to fuse the complementation protein fragments to the termini in the interacting protein that are brought closest together during the interaction. However, even when linker sequences of 15-20 residues are used and the termini of the interacting proteins are more than 40 Å apart, excellent complementation has been observed.

The linkers should primarily be composed of amino acid residues that are small, hydrophilic, are unreactive and constitutes poor protease cleavage sites, e.g. Ser, Thr, Gly.

A preferred embodiment of the invention relates to a nucleic acid encoding any of the conjugates, fragments or fusions proteins described above. In one embodiment, the nucleic acid construct encoding any of the proteins according to the invention described above is a DNA construct. In another embodiment, the nucleic acid construct encoding any of the proteins according to the invention described above is a RNA construct.

As will be evident to the skilled person, the conjugates can be assembled by choice, and the protein is conjugated to the first terminal of the complementation protein in the N-terminal or in the C-terminal. However, as illustrated in the examples, conjugation of the first protein of interest to the N-terminal fragment of GFP shall preferably be to the C-terminal of the N-terminal fragment of GFP. Likewise, conjugation of the second protein of interest to the C-terminal fragment of GFP shall preferably be to the N-terminal of the C-terminal fragment of GFP.

As will be evident from the present examples the protein of interest is a protein, a peptide or a non-proteinaceous partner.

Thus, one embodiment of the present invention relates to a cell wherein the N-terminal fragment of the complementation protein is fused in frame with the first protein of interest.

One embodiment of the present invention relates to a cell, wherein the first protein is fused to the N-terminal of the N-terminal fragment of the complementation protein.

One embodiment of the present invention relates to a cell, wherein the first protein is fused to the C-terminal of the N-terminal fragment of the complementation protein.

One embodiment of the present invention relates to a cell, wherein the C-terminal fragment of the complementation protein is fused in frame with the second protein.

One embodiment of the present invention relates to a cell, wherein the second protein of interest is fused to the N-terminal of the C-terminal fragment of the complementation protein.

One embodiment of the present Invention relates to a cell, wherein the second protein of Interest is fused to the C-terminal of the C-terminal fragment of the complementation protein.

One embodiment of the present invention relates to a cell, wherein the N-terminal fragment of the complementation protein fused in frame to a first protein further comprises a linker sequence between the N-terminal fragment of the complementation protein and the first protein.

One embodiment of the present invention relates to a cell, wherein the C-terminal fragment of the complementation protein fused in frame to a second protein further comprises a linker sequence between the C-terminal fragment of the complementation protein and the second protein.

One embodiment of the present invention relates to a cell, wherein the GFP is EYFP further containing an F64L mutation, wherein X is 172, wherein the first protein fused to the N-terminal fragment of GFP is fused to the C-terminal of the N-terminal fragment of GFP and wherein the second protein fused to the C-terminal fragment of GFP is fused to the N-terminal of the C-terminal fragment of GFP.

One embodiment of the present invention relates to a cell, wherein the GFP is EYFP further containing an F64L mutation, wherein X is 157, wherein the first protein fused to the N-terminal fragment of GFP is fused to the C-terminal of the N-terminal fragment of GFP and wherein the second protein fused to the C-terminal fragment of GFP is fused to the N-terminal of the C-terminal fragment of GFP.

It is not a prerequisite that the conjugates are expressed within the cell, however, it is preferred that the first conjugate is expressed in the cell. Likewise, in a preferred embodiment the second conjugate is expressed in the cell.

In a series of embodiments of the present invention at least one of the conjugates will contain an anchor protein. In the present context, an anchor protein means any and all cellular components that have a defined cellular distribution, preferably genetically encodable cellular components.

It is important, when selecting the anchor, that the nature of the system is taken into account. As an example, some proteins normally require to be phosphorylated or dephosphorylated by enzymes sequestered in the plane of the plasma membrane—for such proteins of interest it is appropriate to choose an anchor component that would be expected to be confined to the plasma membrane, to allow the interacting proteins to be appropriately modified when measuring protein-protein interactions, or to allow measurement of translocation to that location. Thus, in one embodiment, a preferred anchor component that will target the conjugate to the plasma membrane is a protein containing the transmembrane domain of the epidermal growth factor receptor (EGFR), or containing the transmembrane domain of a protein from the integrin protein family, or containing the myristoylation sequence from c-Src (residues 1-14).

In another embodiment, a histone protein is used as the anchor, or a protein normally restricted to nucleoli, for example the p120 nucleolar protein, in order to direct the anchoring conjugate to the nucleus, thereby allowing measurement of protein-protein interactions in the nucleus, or measurement of translocation to the nucleus.

In another embodiment, the anchor protein is chosen from those proteins normally confined to mitochondrial outer or inner membranes for example VDAC, F₀ subunit of ATP-ase, or NADH dehydrogenase. In another embodiment, the anchor protein is chosen from the group of proteins normally confined to the various different regions of Golgi bodies for example TGN38 or ADAM12-L. In another embodiment, the anchor protein is chosen from the group of proteins normally confined to focal adhesion complexes for example P125, FAK, integerin alpha or beta, or paxillin. In another embodiment, the anchor protein is chosen from the group of proteins normally associated with cytoskeletal structures such as F-actin strands or micro tubular bundles for example MAP4, actin binding domain of alpha-actinin (actinPaint), kinesins, myosins or dyniens.

In another embodiment the anchor protein is a protein normally associated with nuclear material or nuclear components, such as histone proteins, including histones H1, H2A, H2B, H3, H4, and variants thereof.

In another embodiment the anchor protein is a protein normally associated with nuclear membrane such as A and B type lamins, or associated with nuclear bodies such as splicing bodies, Cajal bodies, PML nuclear bodies (PML oncogenic domains, PODS), or transcription engines such as RNA polymerase POL-II.

As mentioned, some configurations of the present invention requires spontaneous interaction proteins. Examples of protein domains that may be used to bring about spontaneous interaction are leuzine zippers and coiled-coil domains generally. These domains have been identified in many different proteins, and the properties of self-association predicted with high accuracy from linear sequences of amino acids. Examples of proteins containing leucine zippers are c-Jun and c-Fos. Coiled coil domains and other self interacting domains are found in the Rho kinase ROCK, Bcr, Bcr-Abl, all known AKAPs and in the regulatory subunits PKA. Other spontaneous binding proteins are the binding of JNK to the delta region in c-Jun and the binding of beta-catenin to Tcf4 transciption factor.

The particular utility of stimulus-induced interactions is that in one and the same cell it is possible to switch on a distinctive interaction where previously there was only a very weak interaction or no interaction. This ensures that, in advance, the distinctive interaction is purely a result of specific interaction between the two interaction partners. In the case of conjugates that include a specific anchor protein that locates the fixed conjugate to a specific cellular location, use of a stimulus-induced interaction also guarantees that this interaction will give a signal that is measurable by the assay equipment configured to detect the expected location of the complementation product.

In the embodiments using the interaction partners A and B, the specificity and usefulness of those aspects will depend on interaction partner A and interaction partner B having no measurable affinity for each other in the absence of the interaction stimulus. In one embodiment therefore, interactors A and B are chosen in appropriate pairs among the proteins targeted by immunosuppressants such as, but not limited to, cyclosporin A, Rapamycin and FK506. These proteins include, but are not limited to, FKBP12, FRAP and cyclophilin. In a preferred embodiment, interactors A and B are represented by FKBP12 and a mutated fragment of FRAP (FRB T2098L, ARIAD Pharmaceuticals) and the interaction stimulus this interactor pair is represented by the rapalog AP21967 (ARIAD Pharmaceuticals).

An example of suitable combinations of interaction protein A and interaction protein B and interaction stimulus are listed below:

| Interaction | Interaction | |
|---|---|---|
| partner A | partner B | Interaction stimulus |
| FKBP12 | FRAP | Rapamycin |
| FKBP12 | FRB (T2098L) | AP21967 |

In another embodiment, the ligand-binding domain of a steroid hormone receptor such as, but not limited to, the estrogen receptor is used as both interactor A and B. Such a ligand-binding domain will homo-dimerize upon addition of its cognate hormone ligand (in this case estrogen) (see FIG. 22).

In another embodiment, the full-length or the ligand-binding domain of a steroid hormone receptor is chosen as interactor A whereas interactor B is chosen among the family of steroid hormone receptor co-activators including, but not limited to, SRC-1, GRIP-1, ACTR, AIB-1. As above, cognate hormone is used as interaction stimulus.

One major advantage of the present system is the ability to measure protein-protein interactions as a simple change in light intensity. Thus, one aspect of the invention relates to a method for detecting protein-protein interactions comprising the steps of:
(a) providing a cell comprising at least:
   a first conjugate comprising the first protein of interest and a first terminal complementation protein; and
   a second conjugate comprising the second protein of interest and the second terminal complementation protein;
   wherein said first conjugate has a predominant cellular location that is distinct and spatially separated from where said second conjugate is predominantly located,
(b) determine if complementation partner A and complementation partner B has complemented;
   complementation between the first and second terminal complemenation protein being indicative that the first protein of interest has translocated from the predominant location of this first protein of interest to the cellular location where the second protein of interest is located and that the two proteins of interest interact (FIG. 21).

In the typical use of this method for detecting protein-protein interactions, before step (b) a stimulus know to cause translocation of the first conjugate is applied. This stimulus could be stress, light, a compound or other means. Also, but before adding the translocation stimulus, a compound to be tested is added. If this compound is capable of preventing complementation, It is capable of preventing the interaction between the two proteins of interest, of preventing the complementation as such, or of preventing the translocation of the first conjugate.

An example of this method is to determine the interaction between receptors (dimerization) by having the first protein as one receptor and the second protein as the other receptor (optionally the same receptor: homo-dimerization).

As mentioned above, in a typical configuration of such assay, the second conjugate further comprises an anchor protein (as described in detail above), wherein said anchor protein is anchored in a different cellular location from where said first protein of interest is predominantly located.

Another advantage of the present invention is that it allows translocation to be measured as a change in light intensity. Thus, an aspect of the present invention relates to a method for detecting translocation of a protein comprising the steps of:
(a) providing a cell comprising at least:
   a first conjugate comprising said protein, a first terminal of a complementation protein, and interaction partner A; and
   a second conjugate comprising an anchor protein, the second terminal of the complementation protein, and interaction partner B;
   wherein said first conjugate has a predominant cellular location that is distinct and spatially separated from where said second conjugate is predominantly located and wherein said interaction partner A and interaction partner B bind to each other;
(b) determine if the terminals of the complementation protein has reassembled;
   reassembly (complementation) between the two terminals being indicative that said protein has translocated from the predominant location of the protein to the cellular location where the anchor protein is anchored (FIG. 21).

In the typical use of this method for detecting translocation of a protein, before step (b) a stimulus know to cause translocation of the first conjugate is applied. This stimulus could be stress, light, a compound or other means. Also, but before adding the translocation stimulus, a compound to be tested is added. If this compound is capable of preventing complementation, it is capable of preventing the interaction between the two interaction partners, of preventing the complementation as such, or of preventing the translocation of the first conjugate.

As shown in the examples, some spontaneous fluorescence appears. It is presently hypothesised that this is due to the equilibrium of one or both conjugates between the various cellular locations Thus, when coinciding with the other interaction partner, interaction and consequent complementation occurs. As the complemented protein does not dissociate this will constitute a sink effect (as described in detail above). This effect can, dependent on the system in question, be rather pronounced. The problem can be that the natural shuttling of any naturally translocating protein is exposing the anchored interaction partner construct to a constant supply of binding partner resulting in apparently irreversible binding and premature fluorophore formation.

One way of solving this is to replace the binding interaction partners with interaction partners requiring an interaction stimulus to interact, often referred to as a bridging molecule. Such system provides convenient means of associating e.g. the nuclear fraction of a translocating protein to an anchor protein at any given time.

One example of such interaction pairs is the FKBP12: FRB system (as described in detail above). Thus, one aspect of the invention relates to a method for detecting translocation of a protein comprising the steps of:
(a) providing a cell comprising at least:
   a first conjugate comprising said protein, a first terminal of a complementation protein, and interaction partner A; and
   a second conjugate comprising an anchor protein, a second terminal of the complementation protein, and interaction partner B;
   wherein said first conjugate has a predominant cellular location that is distinct and spatially separated from where said second conjugate is predominantly located,
   wherein said interaction partner A and interaction partner B bind to each other only when an interaction stimulus has been applied;
(b) add an interaction stimulus;
(c) determine if the first and second terminal of the complementation protein has complemented;
   complementation between the two terminals being indicative that said protein has translocated from the predominant location of the protein to the cellular location where the anchor protein is anchored after addition of the interaction stimulus (FIG. 21).

In the typical use of this method for detecting translocation of a protein, before step (b) a stimulus know to cause translocation of the first conjugate is applied. This stimulus could be stress, light, a compound or other means. Also, but before adding the translocation stimulus, a compound to be tested is added. If this compound is capable of prevention complementation, it is capable of preventing the interaction between the two interaction partners, of prevention the complementation as such, or of preventing the translocation of the first conjugate.

As shown in Example 11, using the FRAP system, no spontaneous interaction is observed even though the two conjugates are in the cytoplasm. Thus as discussed above, the efforts in keeping the proteins apart need not to be more than what the natural balance between the location of proteins dictates.

Sometimes measurement of translocation per se is indeed interesting. However, typically translocation is a result of underlying cellular processes. Some of these underlying processes can be utilized to gain information about other processes. For example, when a cell is undergoing apoptosis, the activity of caspase enzymes is increased. With the method and materials of the present innvention it is made possible to detect if the peptide sequence, a consensus cleavage site for caspases has been cleaved (see FIG. 22). The method for determining apoptosis/caspase activity in a cell comprises at least the steps of:
(a) providing a cell comprising at least:
   a first conjugate comprising the first terminal of a complementation protein, interaction partner A, a nuclear localisation sequence (NLS), a Valine-Alanine-Aspartate (VAD) sequence and an anchor protein;
   a second conjugate comprising the second terminal of a complementation protein, interaction partner B and an anchor protein;
wherein the anchor protein of the first conjugate is in a different cellular location from the nucleus, and the anchor protein of the second conjugate is in the nucleus and
wherein interaction partner A, the NLS and the first terminal of the complementation protein are all located on the same side of the VAD sequence and the anchor protein is located on the other side of the NLS;
(b) determine if the complementation protein has complemented
   complementation being Indicative that caspase has cleaved the first conjugate and the free part has translocated to the nucleus, and partner A and complementation partner B has complemented;

A kit for detecting antigens comprising:
(a) a first antibody that binds to the antigen;
(b) a first conjugate comprising a protein binding to the first antibody and the N-terminal of a complementation protein;
(c) a second conjugate comprising a protein binding to the first antibody and the C-terminal of the complementation protein;
   whereby binding of the first and second conjugate to the first antibody will bring the two terminals of the complementation protein so close that a functional protein is formed.

The thoughts of how to construct and test probes useful in the present invention are described below using, as a specific example, GFP as the complementing protein. As will be evident to the skilled person, similar thoughts will aply using other complementing proteins and the complementing fragments thereof.

In general, a probe, i.e. a “GeneX”-GFP fusion or a GFP-“GeneX” fusion, is constructed using PCR with “GeneX”-specific primers followed by a cloning step to fuse “GeneX” in frame with GFP. The fusion may contain a short vector derived sequence between “GeneX” and GFP (e.g. part of a multiple cloning site region in the plasmid) resulting in a peptide linker between “GeneX” and GFP in the resulting fusion protein.

Detailed Stepwise Procedure:

Identifying the sequence of the gene. This is most readily done by searching a depository of genetic information, e.g. the GenBank Sequence Database, which is widely available and routinely used by molecular biologists. In the specific examples below the GenBank Accession number of the gene in question is provided.
Design of gene-specific primers. Inspection of the sequence of the gene allows design of gene-specific primers to be used in a PCR reaction. Typically, the top-strand primer encompasses the ATG start codon of the gene and the following ca. 20 nucleotides, while the bottom-strand primer encompasses the stop codon and the ca. 20 preceding nucleotides, if the gene is to be fused behind GFP, i.e. a GFP-“GeneX” fusion. If the gene is to be fused in front of GFP, i.e. a “GeneX”-GFP fusion, a stop codon must be avoided. Optionally, the full length sequence of GeneX may not be used in the fusion, but merely the part which localizes and redistributes like GeneX in response to a signal.

In addition to gene-specific sequences, the primers contain at least one recognition sequence for a restriction enzyme, to allow subsequent cloning of the PCR product. The sites are chosen so that they are unique in the PCR product and compatible with sites in the cloning vector. Furthermore, it may be necessary to include an exact number of nucleotides between the restriction enzyme site and the gene-specific sequence in order to establish the correct reading frame of the fusion gene and/or a translation initiation consensus sequence. Lastly, the primers always contain a few nucleotides in front of the restriction enzyme site to allow efficient digestion with the enzyme.
Identifying a source of the gene to be amplified. In order for a PCR reaction to produce a product with gene-specific primers, the gene-sequence must initially be present in the reaction, e.g. in the form of cDNA. Information in GenBank or the scientific literature will usually indicate in which tissue(s) the gene is expressed, and cDNA libraries from a great variety of tissues or cell types from various species are commercially available, e.g. from Clontech (Palo Alto), Stratagene (La Jolla) and Invitrogen (San Diego). Many genes are also available in cloned form from The American Type Tissue Collection (Virginia).
Optimizing the PCR reaction. Several factors are known to influence the efficiency and specificity of a PCR reaction, including the annealing temperature of the primers, the concentration of ions, notably Mg²⁺ and K⁺, present in the reaction, as well as pH of the reaction. If the result of a PCR reaction is deemed unsatisfactory, it might be because the parameters mentioned above are not optimal. Various annealing temperatures should be tested, e.g. in a PCR machine with a built-in temperature gradient, available from e.g. Stratagene (La Jolla), and/or various buffer compositions should be tried, e.g. the OptiPrime buffer system from Stratagene (La Jolla).
Cloning the PCR product. The vector into which the amplified gene product will be cloned and fused with GFP will already have been taken into consideration when the primers were designed. When choosing a vector, one should at least consider in which cell types the probe subsequently will be expressed, so that the promoter controlling expression of the probe is compatible with the cells. Most expression vectors also contain one or more selective markers, e.g. conferring resistance to a drug, which is a useful feature when one wants to make stable transfectants. The selective marker should also be compatible with the cells to be used.

The actual cloning of the PCR product should present no difficulty as it typically will be a one-step cloning of a fragment digested with two different restriction enzymes into a vector digested with the same two enzymes. If the cloning proves to be problematic, it may be because the restriction enzymes did not work well with the PCR fragment. In this case one could add longer extensions to the end of the primers to overcome a possible difficulty of digestion close to a fragment end, or one could introduce an intermediate cloning step not based on restriction enzyme digestion. Several companies offer systems for this approach, e.g. Invitrogen (San Diego) and Clontech (Palo Alto).

Once the gene has been cloned and, in the process, fused with the GFP gene, the resulting product, usually a plasmid, should be carefully checked to make sure it is as expected. The most exact test would be to obtain the nucleotide sequence of the fusion-gene.

Testing the Probe

Once a DNA construct for a probe has been generated, its functionality and usefulness may be tested. In a typical scenario, the two construes are individually validated with full length GFP by subjecting it to the following tests:
Transfecting it into cells capable of expressing the probe. The fluorescence of the cell is inspected soon after, typically the next day. At this point, two features of cellular fluorescence are noted: the intensity and the sub-cellular localization.

The intensity should usually be at least as strong as that of unfused GFP in the cells. If it is not, the sequence or quality of the probe-DNA might be faulty, and should be carefully checked.

The sub-cellular localization is an indication of whether the probe is likely to perform well. If it localizes as expected for the gene in question, e.g. is excluded from the nucleus, it can immediately go on to a functional test. If the probe is not localized soon after the transfection procedure, it may be because of overexpression at this point in time, as the cell typically will have taken of very many copies of the plasmid, and localization will occur in time, e.g. within a few weeks, as plasmid copy number and expression level decreases. If localization does not occur after prolonged time, it may be because the fusion to GFP has destroyed a localization function, e.g. masked a protein sequence essential for interaction with its normal cellular anchor-protein. In this case the opposite fusion might work, e.g. if GeneX-GFP does not work, GFP-GeneX might, as two different parts of GeneX will be affected by the proximity to GFP. If this does not work, the proximity of GFP at either end might be a problem, and it could be attempted to increase the distance by incorporating a longer linker between GeneX and GFP in the DNA construct.

If there is no prior knowledge of localization, and no localization is observed, it may be because the probe should not be localized at this point, because such is the nature of the protein fused to GFP. It should then be subjected to a functional test.

In a functional test, the cells expressing the probe are treated with at least one compound known to perturb, usually by activating, the signalling pathway on which the probe is expected to report by redistributing itself within the cell. If the redistribution is as expected, e.g. if prior knowledge tell that it should translocate from location X to location Y, it has passed the first critical test. In this case it can go on to further characterization and quantification of the response.

If it does not perform as expected, it may be because the cell lacks at least one component of the signalling pathway, e.g. a cell surface receptor, or there is species incompatibility, e.g. if the probe is modelled on sequence information of a human geneproduct, and the cell is of hamster origin. In both instances one should identify other cell types for the testing process where these potential problems would not apply.

It this is satisfactory, the conjugates will be constructed with fragments of the complementation protein instead of full-lenght and tested when expressed in the same cell. If no feature of redistribution can be identified, the problem might be as mentioned above, and the probe should be retested under more optimal cellular conditions.

If the probe does not perform under optimal cellular conditions it's back to the drawing board.

Numerous cell systems for transfection exist. A few examples of mammalian cells isolated directly from tissues or organs taken from healthy or diseased animals (primary cells), or transformed mammalian cells capable of indefinite replication under cell culture conditions (cell lines). The term “mammalian cell” is intended to indicate any living cell of mammalian origin. The cell may be an established cell line, many of which are available from The American Type Culture Collection (ATCC, Virginia, USA) or similar Cell Culture Collections. The cell may be a primary cell with a limited life span derived from a mammalian tissue, including tissues derived from a transgenic animal, or a newly established immortal cell line derived from a mammalian tissue including transgenic tissues, or a hybrid cell or cell line derived by fusing different cell types of mammalian origin e.g. hybridoma cell lines. The cells may optionally express one or more non-native gene products, e.g. receptors, enzymes, enzyme substrates, prior to or in addition to the fluorescent probe. Preferred cell lines include, but are not limited to, those of fibroblast origin, e.g. BHK, CHO, BALB, NIH-3T3 or of endothelial origin, e.g. HUVEC, BAE (bovine artery endothelial), CPAE (cow pulmonary artery endothelial), HLMVEC (human lung micro vascular endothelial cells), or of airway epithelial origin, e.g. BEAS-2B, or of pancreatic origin, e.g. RIN, INS-1, MIN6, bTC3, aTC6, bTC6, HIT, or of hematopoietic origin, e.g. primary isolated human monocytes, macrophages, neutrophils, basophils, eosinophils and lymphocyte populations, AML-14, AML-193, HL-60, RBL-1, U937, RAW, JAWS, or of adipocyte origin, e.g. 3T3-L1, human pre-adipocytes, or of neuroendocrine origin, e.g. AtT20, PC12, GH3, muscle origin, e.g. SKMC, A10, C2C12, renal origin, e.g. HEK 293, LLC-PK1, or of neuronal origin, e.g. SK-N-DZ, SK-N-BE(2), HCN-1A, NT2/D1.

The examples of the present invention are based on CHO cells. Therefore, fibroblast derived cell lines such as BALB, NIH-3T3 and BHK cells are preferred.

It is preferred that the heterologous conjugates are introduced into the cell as plasmids, e.g. individual plasmids mixed upon application to cells with a suitable transfection agent such as FuGENE so that transfected cells express and integrate all heterologous conjugates (or GFP fragments) simultaneously. Plasmids coding for each conjugate will contain a different genetic resistance marker to allow selection of cells expressing those conjugates It is also preferred that each of the conjugates also contains a distinct amino add sequence, such as the HA or myc or Flag markers, that may be detected immunocytochemically so that the expression of these conjugates in cells may be readily confirmed. Many other means for introduction of one or both of the conjugates are evenly feasible e.g. electroporation, calcium phosphate precipitate, microinjection, adenovirus and retroviral methods, bicistronic plasmids encoding both conjugates etc.

Throughout the present invention, the term “protein” should have the general meaning. That includes not only a translated protein, a peptide or a protein fragment, but also chemically synthesized proteins. For proteins translated within the cell, the naturally, or induced, post-translational modifications such as glycosylation and lipidation are expected to occur and those products are still considered proteins.

The term “intracellular protein interaction” has the general meaning of an interaction between two proteins, as described above, within the same cell. The Interaction is due to covalent and/or non-covalent forces between the protein components, most usually between one or more regions or domains on each protein whose physico-chemical properties allow for a more or less specific recognition and subsequent interaction between the two protein components involved. In a preferred embodiment, the intracellular interaction is a protein-protein binding.

The recording of the fluorescence will vary according to the purpose of the method in question. In one embodiment the emitted light is measured with various apparatus known to the person skilled in the art. Typically, such apparatus comprises the following components: (a) a light source, (b) a method for selecting the wavelength(s) of light from the source that will excite the luminescence of the luminophore, (c) a device that can rapidly block or pass the excitation light into the rest of the system, (d) a series of optical elements for conveying the excitation light to the specimen, collecting the emitted fluorescence in a spatially resolved fashion, and forming an image from this fluorescence emission (or another type of intensity map relevant to the method of detection and measurement), (e) a bench or stand that holds the container of the cells being measured in a predetermined geometry with respect to the series of optical elements, (f) a detector to record the light intensity, preferably in the form of an image, (g) a computer or electronic system and associated software to acquire and store the recorded information and/or images, and to compute the degree of redistribution from the recorded images.

In a preferred embodiment of the invention, the apparatus system is automated. In one embodiment, the components in (d) and (e) mentioned above comprise a fluorescence microscope. In one embodiment, the component in (f) mentioned above is a CCD camera. In one embodiment, the component in (f) mentioned above is an array of photo multiplier tubes/devices.

In one embodiment of the invention, the actual fluorescence measurements are made in a standard type of fluorometer for plates of micro titer type (fluorescence plate reader).

In one embodiment, the optical scanning system is used to illuminate the bottom of a plate of micro titer type so that a time-resolved recording of changes in luminescence or fluorescence can be made from all spatial limitations simultaneously.

In one embodiment, the image is formed and recorded by an optical scanning system.

A variety of instruments exist to measure light intensity. In one embodiment a fluorescence plate reader is used (e.g. Wallac Victor (BD Biosciences), Spectrafluor (Tecan), Flex station (Molecular Devices), Explorer (Acumen)). In another embodiment an imaging plate readers is used (e.g. FLIPR (Molecular Devices) LeadSeaker (Amersham), VIPR (Molecular Devices)). In another embodiment an automated imager is used like Arrayscan (Cellomics), Incell Analyser (Amersham), Opera (Evotec). In a still further embodiment a confocal fluorescence microscope is used (e.g. LSM510 (Zeiss)).

One particular advantage of the present method is that it can be carried out in a heterogeneous cell population. This avoids inter alia the steps required to get clonal cells. This is achieved by fluorescence activated cell sorting (FACS) prior to testing. One step in that process is removal of the most green cells, that is the cells wherein functional fluorescence is achieved even though the two proteins of interest were not supposed to interact. Another step is removal of the black cells, that is the cells wherein the two heterologous conjugates do not interact e.g. where no or little functional complementation occurs. This could be due to lack of transfection in those cells, a poor expression ratio between the two constructs, or lack of functional expression of either construct. It is presently anticipated that, in both the most green cells and the black cells, the transfection has not taken place as desired, resulting in no, poor, or excessive complementation of the heterologous conjugates. The hereby obtained “medium to low-green” cells are then used in any of the methods described above, or other complementation based methods. The “most green”, “medium green”, “low green” and “black” cells respectively have decreasing levels of fluorescence relative to on another. These levels are predetermined by the skilled artisan in relative proportions

The preferred method for detecting interactions between proteins of interest include an additional FACS. The aim of this second FACS step is to isolate cells with a large dynamic range. The first action In the additional FACS is stimulating the “medium to low-green” FACS cells with the compound that induce interaction between two proteins of interest and thereafter allow sufficient time to pass to let the proteins interact and the fluorescent protein fragments fold and become fluorescent. The next action is to subject them to a second FACS removing the dark cells and removing the medium green cells. The remaining population of cells (most green cells or very green cells (VGC)) will have a low to medium background and are still capable of forming the fluorescent protein upon interaction between the two proteins of interest. When the cells have grown to sufficient number, and a number of generations will have diluted the fluorescence, the cells are ready to use in any of the methods outlined above, e.g. detecting compounds that induce interaction between two proteins of interest and to screen for compounds that interfere with a conditional interaction between two protein components.

In a preferred aspect of the methods, the at least one cell is a mammalian cell.

The term “compound” is intended to indicate any sample, that has a biological function or exerts a biological effect in a cellular system. The sample may be a sample of a biological material such as a sample of a body fluid including blood, plasma, saliva, milk, urine, or a microbial or plant extract, an environmental sample containing pollutants including heavy metals or toxins, or it may be a sample containing a compound or mixture of compounds prepared by organic synthesis or genetic techniques. The compound may be small organic compounds or biopolymers, including proteins and peptides.

In another preferred aspect of the methods, the heterologous conjugates are fusion proteins.

One aspect of the invention relates to multiplexing split fluorophore complementation using different colours. By combining one fluorescent protein fragment with two or more appropriate complementary fragments, it is possible to determine the extent of binding of the first fluorescent protein fragment to either of the two other complementary fragments if the two possible fluorescent complexes have distinct fluorescence excitation or emission characteristics or both. Typically, the first fluorescent protein fragment will be fused to a protein that may bind to either of three other proteins each of them being appropriately fused to distinct complementary fragments. For example, the first fluorescent protein fragment can be a C-terminal fragment of enhanced GFP (EGFP, SEQ ID NO: 1) obtained by splitting EGFP after residue 80. The three complementary fragments can be appropriate N-terminal fragments of EGFP, of EGFP Y66W (SEQ ID NO: 2) and EGFP Y66H (SEQ ID NO: 3), respectively. The three EGFP variants have different spectral characteristics:

| | Excitation max | Emission max |
|---|---|---|
| Fluorescent protein | (nm) | (nm) |
| GFP | 396 | 508 |
| GFP Y66W | 382 | 448 |
| GFP Y66H | 458 | 480 |

Reference: Heim, R., Prasher, D. C., and Tsien, R. Y. (1994) Wavelength mutations and posttranslational autoxidation of green fluorescent protein. Proc. Natl. Acad. Sci U.S.A. 91, 12501-12504.

For example, fluorescent complexes produced by assembling a C-terminal half of GFP (e.g. residues 158 to 238) with corresponding N-terminal halves (e.g. residues 1-157) of GFP, GFP Y66W, or GFP Y66H will have clearly distinct fluorescence characteristics and the relative amounts complexes in mixtures can be calculated.

Not only various colours can be used as exemplified above, but other physical parameters of the fluorophore can be altered e.g. intensity, fluorescent life-time, folding time etc.

The two colour multiplexing has several uses: In most cases, as described above, the protein of interest is linked to the “constant” half of the fluorophore whereas its interaction partners each are linked to “variable” parts of the fluorophore e.g. one that upon fusion gives rise to a green fluorophore and one that gives rise to a blue fluorophore. This can give spatial information, ie the two different interaction partners are in different locations so colour will tell you where your protein of interest is. The interaction partners could also be in the same location so here colour gives you an indication of which interaction partner your protein prefers at any given time. Finally, as a special case of the latter, if your protein's interaction with the different partners is modulated by eg posttranslational modifications, colour can tell you whether your protein is modified or not. These three different setups can be used either as sensors for the physical state of your protein in the broadest possible sense, or they can be used as screening assays where you measure the ability of the test compounds to alter the ratio between the two colour readouts. Finally, colour is only one physical parameter of GFPs. Other physical parameters that can be localised to specific amino acids in the GFP sequence and that are easily detectable, such as absorption spectra, fluorescence lifetime, time for fluorophore maturation etc., could be employed in exactly the same way as colour. The number of different interaction partners need not be limited to two.

One special example shows how this method may be employed as a tool to use spatial information for screening, even if nothing is known about the interaction partners of a particular protein in different cellular locations (or if it has no interaction partners in one or more of those compartments). Cystic Fibrosis is perhaps the most frequent and well-studied protein trafficking disease. The cystic fibrosis transmembrane conductance regulator (CFTR) is a multi-membrane spanning protein that normally functions at the apical plasma membrane of airway epithelial cells as a CI-efflux channel. The most common mutation, (DeltaF508) causes the protein to be retained in the endoplasmic reticulum (ER) and so reduces the amount of CFTR expressed in the plasma membrane of epithelial cells, resulting in decreased CI-efflux from the cells. It would appear from numerous studies that this ER retention defect of DeltaF508 is reversible, and reduced temperature, some small molecules, and induction of “chaperones” allow DeltaF508 to traffic to the plasma membrane and increase CI-permeability. One way of screening for compounds that modify mutant CFTR behaviour employs the split fluorophore/multiple colours concept. You could express the mutant CFTR in cells as a fusion with a zipper fragment fused to the “constant” half of the fluorophore. Expression in the same cell fusions of ER and plasma membrane markers with zippers fused to different colours will reveal when the CFTR mutant reaches the ER, this gives rise to one colour. If CFTR is moved to the PM e.g. by drug stimulus, that would give rise to a different colour. So by screening for compounds that favours the generation of the “PM colour”, you could find molecules that specifically correct the ER retention defect of CF patients.

One aspect of the invention thus relates to a method for generating a library of interacting proteins within living cells consisting of:

1. Introducing into a pool of cells two sets of plasmids, either simultaneously or sequentially, one set of plasmids encoding a library of proteins A each fused to the N-terminal half of the complementing fluorophore and the second set of plasmids encoding a library of proteins B each fused to the C-terminal half of the complementing fluorophore.

2. Sorting the cells by imaging into those where a functional fluorophore has been formed in the right location and those where a functional fluorophore has not been formed or those where a functional fluorophore has been formed but in the wrong cellular compartment, the formation of said functional fluorophore being indicative of an interaction having occurred between proteins A and B within the right cellular compartment.

For example, for a protein moving from one cellular location to another cellular location in response to a stimuli, the binding partners in each location can be identified.

In one embodiment of the invention, the method relates to identification of drugs that will cause disruption of binding between two proteins when located in one cellular compartment or location but not in another cellular compartment or location. This embodiment is carried out essentially as described above with the only difference that instead of sorting the cells based on intensity, the cells are imaged with standard imaging equipment to determine not only if binding has taken place, but also where such binding has occurred.

This system is also useful for screening for fluorophores with novel properties such as those that can be used in the split fluorophore complemensation usages described above. By mutagenisis of both the N- and C-termini of GFP in this system, the system is used to screen in a combinatorial manner for double (or more) mutants of the fluorophore with novel properties. This gives a wider selection to choose from. Furthermore, as the two mutations are in different halves of the molecule, they could be additive or compensatory, or both. Finally, the fact that they have been found using the split fluorophore complementation system immediately means that they can be used as sensors in this system.

The invention will be illustrated more specifically in the following non-limiting examples.

### EXAMPLES

Example 1: Alignment of fluorescent proteins

| GenBank entry | Fluorescent protein |
|---|---|
| P42212 | Aequorea victoria green-fluorescent protein |
| AF372525 | Renilla reniformis green fluorescent protein |
| AY015996 | Renilla muelleri green fluorescent protein |
| AY013824 | Aequorea macrodactyla isolate GFPxm |
| AF384683 | Montastraea cavernosa green fluorescent protein |
| AF401282 | Montastraea faveolata green fluorescent protein |
| AY015995 | Ptilosarcus sp. CSG-2001 green fluorescent protein |
| AF322221 | Anemonia sulcata green fluorescent protein asFP499 |
| AF322222 | Anemonia sulcata nonfluorescent red protein asCP562 |
| AF246709 | Anemonia sulcata GFP-like chromoprotein FP595 |
| AF168419 | DsRed Discosoma sp. fluorescent protein FP583 |
| AF168420 | Discosoma strlata fluorescent protein FP483 |
| AF168421 | Anemonia majano fluorescent protein FP486 |
| AF168422 | Zoanthus sp. fluorescent protein FP506 |
| AF168423 | Zoanthus sp. fluorescent protein FP538 |
| AF168424 | Clavularia sp. fluorescent protein FP484 |

The alignment is presented in FIG. 16.

### Example 2

### Construction of EGFP Complementation Fragment Probes

Anti-parallel leucine zippers (called NZ and CZ) that can bind to each other within prokaryotic and eukaryotic were fused to different fragments of GFP to evaluate the optimal site for splitting GFP for use of such fragments in molecular complementation experiments, including fluorescence reassembly experiments. NZ and CZ leucine zippers were prepared by annealing and ligating phosphorylated oligo nucleotides 2110-2115 (for NZ zipper, see Table 2) or phosphorylated oligo nucleotides 2116-2121 (for CZ zipper), into NcoI-BamHI cut pTrcHis-A vector (commercially available from Invitrogen) producing vector PS1515 (expression vector encoding NZ zipper) or PS1516 (expression vector encoding CZ zipper). The oligos ligated in NZ and CZ annealing mixes 1 produced the coding sequences of the N-terminal parts of the NZ and CZ zippers. The oligos ligated in NZ and CZ annealing mixes 2 produced the coding sequences of the middle parts of the NZ and CZ zippers and the oligos ligated in NZ and CZ annealing mixes 3 produced the coding sequences of the C-terminal parts of the NZ and CZ zippers.

Annealing primer pairs for NZ zipper
NZ annealing mix 1

| | |
|---|---|
| Forward oligo 2110 (1 μM) | 5 μl |
| Reverse oligo 2111 (1 μM) | 5 μl |
| 50 mM Tris-HCl, 10 mM MgCl₂, pH 8.0 | 2 μl |
| H₂O | 8 μl |

NZ annealing mix 2

| | |
|---|---|
| Forward oligo 2112 (1 μM) | 5 μl |
| Reverse oligo 2113 (1 μM) | 5 μl |
| 50 mM Tris-HCl, 10 mM MgCl₂, pH 8.0 | 2 μl |
| H₂O | 8 μl |

NZ annealing mix 3

| | |
|---|---|
| Forward oligo 2114 (1 μM) | 5 μl |
| Reverse oligo 2115 (1 μM) | 5 μl |
| 50 mM Tris-HCl, 10 mM MgCl₂, pH 8.0 | 2 μl |
| H₂O | 8 μl |

Each of the annealing mixes were heated at 80° C. for 2 minutes on a pre-heated Hybaid OmniGene PCR machine which was subsequently turned off and allowed to cool to room temperature (about 10 min). The fragments were subsequently put on ice.

Annealing primer pairs for CZ zipper
CZ annealing mix 1

| | |
|---|---|
| Forward oligo 2116 (1 μM) | 5 μl |
| Reverse oligo 2117 (1 μM) | 5 μl |
| 50 mM Tris-HCl, 10 mM MgCl₂, pH 8.0 | 2 μl |
| H₂O | 8 μl |

CZ annealing mix 2

| | |
|---|---|
| Forward oligo 2118 (1 μM) | 5 μl |
| Reverse oligo 2119 (1 μM) | 5 μl |
| 50 mM Tris-HCl, 10 mM MgCl₂, pH 8.0 | 2 μl |
| H₂O | 8 μl |

CZ annealing mix 3

| | |
|---|---|
| Forward oligo 2120 (1 μM) | 5 μl |
| Reverse oligo 2121 (1 μM) | 5 μl |
| 50 mM Tris-HCl, 10 mM MgCl₂, pH 8.0 | 2 μl |
| H₂O | 8 μl |

Each of the annealing mixes were heated at 80° C. for 2 minutes on a pre-heated Hybaid OmniGene PCR machine which was subsequently turned off and allowed to cool to room temperature (about 10 min). The fragments were subsequently put on ice.

Restriction Digestion of pTrcHis-A Prokaryotic Expression Vector

The pTrcHis-A prokaryotic expression vector, cut with NcoI and BamHI restriction enzymes and gel purified, was used for cloning the prepared NZ and CZ leucine zipper coding sequences:

Restriction digestion of pTrcHis-A vector

| | |
|---|---|
| pTrcHis-A (1 μg/μl) | 2 μl |
| NcoI (10 U/μl) | 1 μl |
| NheI (5 U/μl), optional | 0.5 μl |
| BamHI (20 U/μl) | 1 μl |
| 100x BSA | 0.4 μl |
| 10x NEB (New England Biolabs, NEB) BamHI buffer | 3 μl |
| H₂O | 23 μl |
| Calf intestinal phosphatase (optional, last 20 min only) | 0.5 μl |

The vector was digested for about 1 hour at 37° C. and purified by agarose gel electrophoresis. The desired vector fragment was recovered from the gel using the QIAquick Gel Extraction kit (spin columns) from Qiagen and recovered in 50 μl of elution buffer. NheI, which cuts between NcoI and BamHI, was included to minimise the amounts of uncut and self-ligating vector.

Ligation and Transformation of Annealed NZ Oligo Pairs

Each of the three NZ annealing mixtures 1-3 was diluted 50-fold (1 μl of mixture in 50 μl of H₂O) and mixed and ligated into the cut vector as follows (three hours at 20-24° C.):

Ligation of NZ zipper fragments into pTrcHis-A vector

| | |
|---|---|
| Annealing mix 1 | 1 μl |
| Annealing mix 2 | 1 μl |
| Annealing mix 3 | 1 μl |
| 10x T4 DNA ligase buffer (New England Biolabs) | 1 μl |
| T4 DNA ligase (400 U/μl, New England Biolabs) | 0.5 μl |
| pTrcHis-A (NcoI + BamHI cut) | 0.5 μl |
| H₂O | 5 μl |

Alternatively, the fragments in NZ annealing mixes 1, 2, and 3 can be ligated in absence of vector and purified by agarose gel electrophoresis before being ligated into the NcoI-BamHI cut vector. The annealed and ligated oligo nucleotides from annealing mixes 1-3 had single stranded terminal overhangs that were compatible with the overhangs that were generated by NcoI and BamHI restriction digestion of pTrcHis-A. After ligation of the fragment into cut pTrcHis-A, the NcoI and BamHI sites were regenerated.

Following ligation into the vector, 2 μl of the ligation mixture was transformed into 50 μl of One Shot TOP10 chemically competent E. coli cells (Invitrogen) following the manufacturers protocol. The ligation can be performed using different amounts or volumes fragments and buffers. The inserted DNA sequence (SEQ ID NO: 7) and the encoded NZ zipper peptide (SEQ ID NO: 8) are as follows:

```
      M A G G T G S G A L K K E L Q A
CCATGGCCGGTGGTACCGGTTCCGGTGCCCTGAAGAAGGAGCTGCAGGCC
  N K K E L A Q L K W E L Q A L K K
AACAAGAAGGAGCTGGCCCAGCTGAAGTGGGAGCTGCAGGCCCTGAAGAA
    E L A Q * D
GGAGCTGGCCCAGTAGGATCC
```

The Gly-Gly-Thr-Gly-Ser-Gly amino acid sequence in the terminus is part of the linker sequence that was inserted between the NZ zipper peptide and the N-terminal fragments of EGFP (NtermEGFP). The zipper sequence in the NtermEGFP-NZ fusion protein is also Gly-Gly-Thr-Gly-Ser-Gly with the Gly-Gly-Thr-Gly coding sequence being repeated in the NtermEGFP reverse amplification primers 2129, 2130, and 2131 (Table 3). Underlined are the unique NcoI (CCATGG), AgeI (ACCGGT) and BamHI (GGATCC) sites used for cloning of the zipper peptide into pTrcHis-A and the NtermEGFP-NZ fragments into the NZ zipper vector PSI515 (see below). The asterisk (*) shows a stop codon.

Ligation and Transformation of Annealed CZ Oligo Pairs

Each of the three CZ annealing mixtures 4-6 was diluted 50-fold (1 μl of mixture in 50 μl of H₂O) and mixed as follows:

Ligation of CZ zipper fragments into pTrcHis-A vector

| | |
|---|---|
| CZ annealing mix 1 | 1 μl |
| CZ annealing mix 2 | 1 μl |
| CZ annealing mix 3 | 1 μl |
| 10x T4 DNA ligase buffer (New England Biolabs) | 1 μl |
| T4 DNA ligase (400 U/μl, New England Biolabs) | 0.5 μl |
| pTrcHis-A (NcoI + BamHI cut) | 0.5 μl |
| H₂O | 5 μl |

Alternatively, the fragments in CZ annealing mixes 1, 2, and 3 can be ligated in absence of vector and purified by agarose gel electrophoresis before being ligated into the NcoI-BamHI cut vector. The annealed and ligated oligo nucleotides from annealing mixes 1-3 had single stranded terminal overhangs that were compatible with the overhangs that were generated by NcoI and BamHI restriction digestion of pTrcHis-A. After ligation of the fragment into cut pTrcHis-A, the NcoI and BamHI sites were regenerated.

Following ligation into the vector, 21 μl of the ligation mixture were transformation into 50 μl of One Shot TOP10 chemically competent E. coli cells (Invitrogen) following the manufacturers protocol. The ligation can be performed using different amounts or volumes fragments and buffers. The inserted DNA sequence (SEQ ID NO: 9) and the encoded CZ zipper peptide (SEQ ID NO: 10) are as follows:

~~~M~~A~~S~~E~~Q~~L~~E~~K~~K~~L~~Q~~A~~L~~E~~K~~K
CCATGGCCAGCGAGCAGCTGGAGAAGAAGCTGCAGGCCCTGGAGAAGAAG
~L~~A~~Q~~L~~E~~W~~K~~N~~Q~~A~~L~~E~~K~~K~~L~~A~~Q
CTGGCCCAGCTGGAGTGGAAGAACCAGGCCCTGGAGAAGAAGCTGGCCCA
~~G~~G~~T~~G~~*
GGGCGGCACCGGTTAGGATCC

The Gly-Gly-Thr-Gly amino acid sequence in the terminus is part of the linker sequence that was inserted between the CZ zipper peptide and the C-terminal fragments of EGFP (CtermEGFP). The zipper sequence in the CZ-CtermEGFP fusion protein is also Gly-Gly-Thr-Gly with the Thr-Gly coding sequence being repeated in the CtermEGFP forward amplification primers 2133, 2134, and 2135 (Table 3). Underlined are the unique NcoI (CCATGG), AgeI (ACCGGT) and BamHI (GGATCC) sites used for cloning of the zipper peptide into pTrcHis-A and the CZ-CtermEGFP fragments into the CZ zipper vector PS1516 (see below). The asterisk (*) shows a stop codon.

### Example 3

### E. coli Colony PCR Screen, Plasmid Miniprep and DNA Sequencing

The transformed bacteria were plated on Luria Broth (LB) agar plates containing 100 μg/ml of carbenicillin as selection (present in used E. coli media). To quickly identify transformants containing the desired NZ or CZ constructs, colony PCR screening was performed using oligos 2110 (5′ forward NZ oligo) and 2115 (3′ reverse NZ oligo) or using oligos 2116 (5′ forward CZ oligo) and 2121 (3′ reverse CZ oligo):

Per sample (15 μl reaction volume)

| | |
|---|---|
| 10x Taq polymerase buffer (Perkin Elmer) | 1.5 μl |
| dNTP (5 mM nucleotide, each) | 0.3 μl |
| 50 mM MgCl₂ | 0.6 μl |
| Dimethyl sulphoxide (DMSO) | 0.3 μl |
| Taq polymerase (Perkin Elmer) | 0.2 μl |
| 5′ forward primer (10 μM) | 0.5 μl |
| 3′ reverse primer (10 μM) | 0.5 μl |
| H₂O | 6.1 μl |
| Transformant resuspended in H₂O | 5.0 μl |

Cycling Parameters (RoboCycler Gradient 96, Stratagene)

Initial denaturation at 94° C. for 3 min followed by 25 cycles of (all steps of 1 min): Denaturation at 94° C., primer annealing at 53° C. and primer extension at 72° C. Finally, an additional extension step at 72° C. was included (5 min). 16 NZ transformants and 16 CZ transformants were screened. PCR fragments having the expected product sizes of about 120 base pairs were amplified from 14 NZ clones and 15 CZ clones, as determined by agarose gel electrophoresis analysis.

Three of the positive colonies were picked from each transformation (NZ and CZ) and used to inoculate 5 ml of liquid LB medium. After culturing at 37° C. over night, plasmid DNA was purified by mini preparations using the QIAprep kit from Qiagen.

Plasmids containing correct NZ (PS1515) or CZ (PS1516) fragment inserts were identified by DNA sequencing on an ABI PRISM model 377 DNA sequencer using forward sequencing primer 1282.

### Example 4

### Prokaryotic Expression Vectors Encoding Fusion Proteins of EGFP Fragment and Zipper

The DNA sequences encoding the NZ and CZ zippers in the prokaryotic expression vectors PS1515 and PS1516, respectively, can be fused to DNA sequences encoding desired EGFP fragments (N-terminal fragments of EGFP are called NtermEGFP and C-terminal fragments of EGFP are called CtermEGFP) or other fragments using the unique AgeI restriction sites appropriately located in linker sequences in either the 5′ end (as in the NZ vector PSI515) or in the 3′ end (as in the CZ vector PS1516) of the leucine zipper coding sequence in combination with either of the unique NcoI or BamHI sites used for cloning the zipper coding fragments (DNA and amino acid sequences are shown above). The general structures of the fusion protein coding sequences are shown in FIG. 1.

For example, to prepare a prokaryotic expression vector encoding a fusion protein consisting of NZ zipper N-terminally fused to an NtermEGFP fragment (that is, fused to the C terminal of the NtermEGFP fragment), e.g. residues 1-172 (NtermEGFP172), this region of the EGFP coding sequence in the commercial expression vector pEGFP-C1 (Clontech) was amplified by PCR using forward oligo 2128 (containing a unique NcoI site) and reverse oligo 2131 (containing a unique AgeI site) in accordance with Table 3.

Per sample (50 μl reaction volume)

| | |
|---|---|
| 10X Pfu polymerase buffer (Stratagene) | 5.0 μl |
| dNTP (5 mM nucleotide, each) | 1.0 μl |
| Pfu Hot Start polymerase (Stratagene) | 1.0 μl |
| 5′ forward primer (10 μM) | 1.0 μl |
| 3′ reverse primer (10 μM) | 1.0 μl |
| pEGFP-C1 vector (10 ng/μl) | 2.0 μl |
| H₂O | 39.0 μl |

Cycling Parameters (Hybaid OmniGene PCR Machine)

Initial denaturation at 94° C. for 3 min followed by 25 cycles of (all steps of 1 min): Denaturation at 94° C., primer annealing at 53° C. and primer extension at 72° C. Finally, an additional extension step at 72° C. was included (5 min).

The PCR fragment encoding the desired EGFP fragment, e.g. the above mentioned fragment composed of residues 1-172, with appropriately engineered terminal restriction sites contained in the primer sequences was then gel purified as described above cut with NcoI and AgeI or AgeI and BamHI and ligated into the constructed NZ or CZ prokaryotic leucine zipper expression vectors PS1515 or PS1516 cut with the same enzymes and gel purified:

Restriction digestion of NtermEGFP and CtermEGFP PCR fragments

| EGFP fragment (gel purified) | 26 μl |
|---|---|
| NcoI (10 U/μl) or BamHI (20 U/μl) | 0.5 μl |
| AgeI (10 U/μl) | 1.0 μl |
| 10X New England Biolabs buffer 2 | 3 |

Restriction digestion of NZ (PS1515) and CZ (PS1516) vectors

| Vector (1 μg/μl) | 1.0 μl |
|---|---|
| NcoI (10 U/μl) or BamHI (20 U/μl) | 0.33 μl |
| AgeI (10 U/μl) | 0.66 μl |
| 10X New England Biolabs buffer 2 | 1 |
| H₂O | 7 |

All enzymes were from New England Biolabs. The DNA preparations were digested for 1 hour at 37° C. and gel purified.

Ligation of EGFP fragments into cut PS1515 or PS1516 vector

| | |
|---|---|
| Cut and purified vector | 2 μl |
| Cut and purified NtermEGFP or CtermEGFP | 4 μl |
| fragment | |
| 10X T4 DNA ligase buffer (New England Biolabs) | 1 μl |
| T4 DNA ligase (400 U/μl, New England Biolabs) | 0.5 μl |
| H₂O | 2.5 μl |

Ligation proceeded for 30 min at 22° C. after which 2 μl of each ligation mixture were transformed into 50 μl of One Shot TOP10 chemically competent E. coli cells (Invitrogen). The transformed cells were plated on LB plates containing carbenicillin and plasmids were prepared from two colonies from each transformation as described above.

### Example 5

### EGFP Based Reassembly in E. coli

Plasmids that expressed functional NtermEGFP-NZ or CZ-CtermEGFP complementation constructs were identified by co-transforming 10 μl of One Shot TOP10 chemically competent E. coli cells (Invitrogen) with 1 μl of each of appropriately matched NtermEGFP-NZ or CZ-CtermEGFP plasmids (i.e., plasmids that express EGFP fragments, said fragments are truncated after (NtermEGFP fragments) or before (CtermEGFP fragments) the same splitting site and plating the co-transformed cells on LB plates containing carbenicillin and 5 mM of isopropyl-β-thiogalactoside (IPTG).

The transformed cells were grown over night at 37° C. E. coli colonies that were green fluorescent because of EGFP based reassembly were visible on the agar plate without magnification about 10-20 hours after transfection (the fluorescence developed further during storage of the plates at 5° C. for one or more days) when illuminated with a blue light source (Fiberoptic-Heim LQ2600) and viewed through yellow filter glasses.

Functional complementation was clearly visible in cells co-transformed with complementation constructs based on splits between either residues 157 and 158 or between residues 172 and 173 and the DNA sequences of expression vectors that produced functional NtermEGFP-NZ or CZ-CtermEGFP complementation fragments (named PS1594, PS1595, PS1596, PS1597, see Table 4) were verified by DNA sequencing using primer 1282 as previously described.

Surprisingly, the E. coli colonies of cells co-transformed with the vectors expressing the EGFP complementation fragments with split in the Ile171-Ser175 loop (namely between residues 172 and 173, vectors PS1595 and PS1597) were significantly more fluorescent than the colonies of cells that were co-transformed with vectors expressing EGFP complementation fragments that were split in the Ala154-Gly160 loop (namely between residues 157 and 158, vectors PS1594 and PS1596).

Functional complementation was not clearly visible in cells co-transformed with complementation constructs based on a split between residues 144 and 145. DNA sequencing confirmed that expression vectors PS1614 and PS1615 encoded the correct NtermEGFP-NZ and CZ-CtermEGFP reassembly fragments, respectively.

### Example 6

### Eukaryotic Expression Vectors Encoding Fusion Proteins of EGFP Fragment and Zipper

Because of the low fluorescence signal produced by the complementation fragments based on the 144/145 split fragments, only the complementation fragments that were based on splits at residues 157/158 or 172/173 were transferred to an eukaryotic expression system to permit evaluation of fragment complementation in mammalian cells.

NtermEGFP-NZ fragments in PS1596 and PS1597, and CZ-CtermEGFP fragments in PS1594 and PS1595, are flanked by an NcoI site 5′ to the start codons and a BamHI site 3′ to the stop codons. The fragments were transferred as blunt-ended NcoI/BamHI fragments into mammalian expression vectors cut with Eco47III/BamHI. To select for stable expression of both an NtermEGFP-NZ and a CZ-CtermEGFP expressing plasmid, the expression vectors for NtermEGFP-NZ fragments and CZ-CtermEGFP fragments contain selection markers for neomycin/geneticin/G418 and zeocin, respectively.

Plasmids PS1594, PS1595, PS1596, and PS1597 were cut with NcoI restriction enzyme, blunt-ended with Klenow fragment, gel purified, cut with BamHI and gel purified as described below.

Restriction digestion of NtermEGFP-NZ and CZ-CtermEGFP
prokaryotic expression vectors

| | |
|---|---|
| PS1594, PS1595, PS1596, or PS1597 (1 μg/μl) | 1 μl |
| NcoI (10 U/μl, from New England Biolabs) | 1 μl |
| 10X buffer 4 (NEB) | 3 μl |
| H₂O | 25 μl |

The plasmids were digested for about 1 hour at 37° C. 1 μl of 1 mM dNTP mix and 1 unit of Klenow fragment (New England Biolabs) were added and the reactions were incubated 30 minutes at room temperature. The linear plasmid fragments were purified by agarose gel electrophoresis and recovered from the gel using the QIAquick Gel Extraction kit (spin columns) from Qiagen and recovered in 50 μl of elution buffer. 5 μl BamHI buffer (New England Biolabs) and 10 units BamHI enzyme were added. The plasmids were digested for about 1 hour at 37° C. The desired plasmid fragments were purified by agarose gel electrophoresis and recovered from the gel using the QIAquick Gel Extraction kit (spin columns) from Qiagen and recovered in 50 μl of elution buffer.

To stably co-express NtermEGFP-NZ and CZ-CtermEGFP fragments in the same mammalian cell, mammalian expression vectors carrying different selection markers were required. To obtain this, the kanamycin/neomycin selection marker on the expression vector pEGFP-C1 was replaced with a zeocin resistance marker resulting in the plasmid referred to as PS0609.

Replacement of Kanamycin/Neomycin Marker on pEGFP-C1 with Zeocin Marker.

pEGFP-C1 was digested with AvrII, which excises the kanamycin/neomycin selection marker, and following gel purification, the vector fragment was ligated with an approximately 0.5 kbp AvrII fragment encoding zeocin resistance. This fragment was isolated by PCR amplification of the zeocin selection marker on plasmid pZeoSV (Invitrogen) using primers 9655 and 9658 (see Table 2). Both primers contain AvrII cloning sites and flank the zeocin resistance gene on plasmid pZeoSV including its E. coli promoter. The top primer 9658 spans the AseI site at the beginning of zeocin, which can be used to determine the orientation of the AvrII insert relative to the SV40 promoter which drives resistance in mammalian cells. The resulting plasmid is referred to as PS0609.

Plasmids pEGFP-C1 (Clontech) and its zeocin-resistant derivative PSO0609 were cut with Eco47III restriction enzyme, gel purified, cut with BamHI and gel purified as described below. These steps excise EGFP and leave the rest of the vectors intact.

Restriction digestion of eukaryotic expression vectors

| | |
|---|---|
| pEGFP-C1 or PS0609 DNA (1 μg/μl) | 0.5 μl |
| Eco47III (10 U/μl, from Promega) | 1 μl |
| 10X buffer D (Promega) | 3 μl |
| H₂O | 25.5 μl |

The plasmids were digested for about 1 hour at 37° C. The linear plasmid fragments were purified by agarose gel electrophoresis and recovered from the gel using the QIAquick Gel Extraction kit (spin columns) from Qiagen and recovered in 50 μl of elution buffer. 5 μl BamHI buffer (New England Biolabs) and 10 units BamHI enzyme were added. The plasmids were digested for about 1 hour at 37° C. The desired vector fragments were purified by agarose gel electrophoresis and recovered from the gel using the QIAquick Gel Extraction kit (spin columns) from Qiagen and recovered in 50 μl of elution buffer.

Ligation of NtermEGFP-NZ fragments into pEGFP-C1 and
CZ-CtermEGFP fragments into PS0609

| | |
|---|---|
| Cut and purified vector fragment | 1 μl |
| Cut and purified NtermEGFP-NZ or CZ-CtermEGFP | 3 μl |
| fragment | |
| 10X T4 DNA ligase buffer (New England Biolabs) | 1 μl |
| T4 DNA ligase (400 U/μl, New England Biolabs) | 0.5 μl |
| H2O | 5 μl |

Ligation reactions were incubated at 16° C. overnight. 3 μl were transformed into One Shot TOP10 chemically competent E. coli cells (Invitrogen) and transformants were selected on imMedia with kanamycin or ImMedia with zeocin (both from Invitrogen) for pEGFP-C1 and PS0609 derivatives, respectively.

4 transformants from each transformation plate were picked in imMedia medium with appropriate selection (kanamycin or zeocin) and grown at 37 degrees C. for 6 hours. Plasmid DNA was isolated by the QIAprep spin column method (Qiagen) and analysed by restriction digests with AseI and MluI. The DNA sequences of the Inserts were finally verified by sequencing as described above. The resulting plasmids were named PS1557, PS1558, PS1559, and PS1560 (Table 4).

### Example 7

### EGFP Based Reassembly in Mammalian Cells

To establish cells lines that express EGFP fragment/zipper fusion proteins, CHO-hIR cells were transfected with plasmid pairs resulting in two cell lines 1) CHO-hIR PS1559+PS1557, and 2) CHO-hIR PS1560+PS1558. The CHO-hIR cell line consists of CHO-Kᵢ (ATCC CCL-61) cells that have been stably transfected with the human insulin receptor ((hIR, GenBank Acc# M10051) as described in: Hansen, B. F., Danielsen, G. M., Drejer, K., Sorensen, A. R., Wiberg, F. C., Klein, H. H., Lundemose, A. G. (1996) Sustained signalling from the insulin receptor after stimulation with insulin analogues exhibiting increased mitogenic potency. Biochem. J. Apr 1; 315 (Pt 1):271-279)<. The selection marker for the vector is methotrexate (MTX). The hIR expression is very stable in the CHO-hIR cells, without selection pressure, because of the insulin-sensitivity of the cell line and a very stable phenotype can be maintained without selection pressure.

Stable cells were obtained by cell growth in selection medium containing Geneticin and Zeocin.

CHO-hIR cells were transfected using Fugene (Roche) according to the manufacturer's instructions. The day after transfection, cells were examined for transient expression, split 1:10 and exposed to selection medium (growth medium supplemented with 500 μg/ml geneticin (Invitrogen) and 1 mg/ml zeocin (Cayla). The cells lines were stable after 2-3 weeks of culture in selection medium.

The growth medium used was NUT.MIX F-12 (Ham's) with GLUTAMAX-1 (Gibco/Invitrogen) supplemented with 10% fetal bovine serum (JRH Biosciences) and 1% Penicillin-Streptomycin (10,000 IU/ml, Gibco/Invitrogen). The CHO-hIR cells were cultured in growth medium, and split 1:4 to 1:16 twice a week according to standard cell culture protocols. The CHO-hIR PS1559+PS1557 and CHO-hIR PS1560+PS1558 were treated likewise, except that the growth medium was supplemented with 500 μg/ml geneticin (Invitrogen) and 1 mg/ml zeocin (Cayla) at all times.

Images of three CHO-hIR cell lines separately transfected with pEGFP-C1 (expressing EGFP with a short C-terminal extension), PS1559+PS1557 (expressing EGFP complementation fragments split at 157-158, NtermEGFP157-NZ+CZ-CtermEGFP158) and with PS1560+PS1558 (expressing EGFP complementation fragments split at 172-173, NtermEGFP172-NZ+CZ-CtermEGFP173) were collected 1 day, 2 days and 10 days after transfection to assess the relative brightness of cells expressing the different complementation constructs. Images were collected on a Nikon Diaphot 300 equipped for epifluorescence work. Light source for epifluorescence was a Nikon 100 W Hg arc lamp, coupled to the microscope through a custom quartz fibre illuminator (TILL Photonics GmbH, Planegg, Germany). Excitation light passed through a 450-490 nm bandpass filter (Delta Light and Optics, Lyngby, Denmark) and was directed to the specimen via a Chroma 72100 505 nm cut-on dichroic mirror (Chroma Technology, Brattleboro, Vt., USA). A x40 NA1.3 oil immersion lens was used for all images. Emitted light passed through a 540-550 bandpass filter (Chroma) to a Hammamatsu Orca ER camera. All images were collected with 50 millisecond exposure time, chosen to ensure non-saturation of images for even the brightest (EGFP-expressing) cells in each optical field (maximum pixel count<4095). Imaging software used to acquire images on this system was IPLab for Windows (Scanalytics, USA).

Presentation and Analysis of Images

The microscope images were analysed using the ImageJ software package, the public domain image analysis software written by Wayne Rasband of the US National Institute of Health (http://rsb.info.nih.gov/ij) and the data analysis was performed in Microsoft Excel. The images shown in FIG. 2 are of fluorescent CHO-hIR cells co-transfected with different NtermEGFP-NZ and CZ-CtermEGFP expression vectors or transfected with pEGFP-C1. The images are scaled individually to visualise the cells and the fluorescence distribution within them. Because of this scaling, the relative fluorescence levels cannot be compared between the images. When the same images are scaled identically they appear as in FIG. 3 and it is apparent that the cells that are transfected with complementation constructs that are based on a split between residues 172 and 173 are significantly more fluorescent than the cells that are transfected with complementation constructs that are based on a split between residues 157 and 158. However, the cells transfected with the pEGFP-C1 construct show significantly stronger fluorescence on day 2.

The same images were analysed for background and maximum fluorescence intensities using the ImageJ software package (FIG. 4). From the figure, it is clear that a split between residues 172 and 173, and probably anywhere else in this loop, is greatly superior to a split between residues 157 and 158 and probably also to splits anywhere else in this loop.

### Example 8

### Eukaryotic Expression Vectors Encoding EYFP and EYFP Variant F64L Fragment/Zipper Fusion Proteins

Mutagenesis of the eukaryotic NtermEGFP-NZ expression vectors PS1559 (NtermEGFP157-NZ) and PS1560 (NtermEGFP172-NZ) into the corresponding N-terminal EYFP (SEQ ID NO: 5) fragment (NtermEYFP-NZ) variants and mutagenesis of the eukaryotic CtermEGFP expression vectors PS1557 (CZ-CtermEGFP158) and PS1558 (CZ-CtermEGFP173) into the corresponding C-terminal EYFP fragment (CZ-CtermEYFP) variants was accomplished by site directed mutagenesis using the QuickChange kit and by following the manufacturers instructions (Stratagene). Primers 2333 and 2334 were used to convert expression vectors PS1559 (NtermEGFP157-NZ) and PS1560 (NtermEGFP172-NZ) into N-terminal EYFP fragment expression vectors PS1639 (NtermEYFP157-NZ) and PS1642 (NtermEYFP172-NZ). The introduced mutations were: L64F:T65G:V68L:S72A. Furthermore, primers 2335 and 2336 were used to convert expression vectors PS1559 (NtermEGFP157-NZ) and PS1560 (NtermEGFP172-NZ) into F64L mutated N-terminal EYFP fragment expression vectors PS1640 (NtermE[F64L]YFP157-NZ) and PS1641 (NtermE[F64L]YFP172-NZ). The introduced mutations were: T65G:V68L:S72A. Accordingly, the expressed NtermEYFP fragments have the following amino acid sequences (only residues 64-72 are shown):

| | 64 | 65 | 66 | 67 | 68 | 69 | 70 | 71 | 72 |
|---|---|---|---|---|---|---|---|---|---|
| NtermEGFP | L | T | Y | G | V | Q | C | F | S |
| (template) | | | | | | | | | |
| NtermEYFP | F | G | Y | G | L | Q | C | F | A |
| (L64F:T65G:V68L:S72A) | | | | | | | | | |
| NtermE[F64L]YFP | L | G | Y | G | L | Q | C | F | A |
| (T65G:V68L:S72A) | | | | | | | | | |

Finally, primers 2337 and 2338 were used to convert expression vectors PS1557 (CZ-CtermEGFP158) and PS1558 (CZ-CtermEGFP173) into C-terminal EYFP fragment expression vectors PS1637 (CZ-CtermEYFP158) and PS1638 (CZ-CtermEYFP173) by introducing a T203Y mutation. All sequences were verified by DNA sequencing of the vectors and all primer sequences are shown in Table 2.

### Example 9

### EYFP Based Reassembly in Mammalian Cells

The constructed EYFP based split fluorescent protein expression vectors PS1637 to PS1642 described above were investigated in mammalian cells in parallel with the EGFP based split fluorescent protein expression vectors PS1557 to PS1560 described in Example 7 and using the same experimental set-up (including the same filter set) and procedures (including the image analysis procedure) except that all images were produced using 10 ms exposure times instead of 50 ms exposure times, because of the increased brightness of the probes, and a 20× objective was used instead of a 40× objective to image more cells. Other appropriate filter sets could have been used. The images are taken the day after transfection (day 1).

It is apparent from the identically scaled fluorescence images of the transfected cells (FIG. 6) that the split site between residues 172 and 173 is again shown to be superior to the split site between residues 157 and 158. Furthermore, it is apparent that complementation based on EYFP fragments is superior to complementation based on EGFP fragments. Surprisingly, introduction of the F64L mutation from EGFP into the N-terminal EYFP fragments further greatly enhanced the fluorescence of the complementing fragments. As can be seen from the images, the positive effects of using the optimal splitting site (between residues 172 and 173) using the optimal fluorescent protein colour variant (EYFP) and introducing the F64L folding mutation into the NtermEYFP fragment, are additive. Quantification of these observation was done by analysing the images shown in FIG. 6 and the numeric out-put is presented in FIG. 7.

Effects of colour (yellow better):

| Good | | Better |
|---|---|---|
| EGFP | vs | EYFP |
| NtermEGFP157-NZ + | vs | NtermEYFP157-NZ + |
| CZ-CtermEGFP158 | | CZ-CtermEYFP158 |
| NtermEGFP172-NZ + | vs | NtermEYFP172-NZ + |
| CZ-CtermEGFP173 | | CZ-CtermEYFP173 |

Effects of split site (172/173 better):

| Good | | Better |
|---|---|---|
| NtermEGFP157-NZ + | vs | NtermEGFP172-NZ + |
| CZ-CtermEGFP158 | | CZ-CtermEGFP173 |
| NtermEYFP157-NZ + | vs | NtermEYFP172-NZ + |
| CZ-CtermEYFP158 | | CZ-CtermEYFP173 |
| NtermE[F64L]YFP157-NZ + | vs | NtermE[F64L]YFP172-NZ + |
| CZ-CtermEYFP158 | | CZ-CtermEYFP172 |

Effects of F64L (+F64L better):

| Good | | Better |
|---|---|---|
| NtermEYFP157-NZ + | vs | NtermE[F64L]YFP157-NZ + |
| CZ-CtermEYFP158 | | CZ-CtermEYFP158 |
| NtermEYFP172-NZ + | vs | NtermE[F64L]YFP172-NZ + |
| CZ-CtermEYFP173 | | CZ-CtermEYFP173 |

It is interesting to note, that the optimal constructs (NtermE[F64L]YFP172-NZ and CZ-CtermE[F64L]YFP173) when re-assembled is nearly as intense as EYFP itself. The great increase in fluorescence intensity is important in many types of quantitative cell analyses (e.g. high through-put screening and microscopy) to increase the signal to noise rations, to facilitate detection of low amounts of probes in vivo or in vitro, etc.

Mixing NtermEYFP with CtermEGFP or NtermEGFP with CtermEYFP fragments can also produce functional fluorescent complexes, potentially of different colours (FIGS. 8 and 9). Fragments having overlapping sequences are also functional and may be very attractive in e.g. functional cloning systems where highly flexible linkers sequences are required due to the very diverse nature of the fusion partners. The overlapping fragments permit either of the fusion partners to have a long linker sequence (FIG. 8, quantified in FIG. 9).

### Example 10

### Construction of PS1769, 1767, 1771, 1768

Plasmid PS1769 encodes a fusion of NtermE[F64L]YFP172 and FKBP, connected by a linker sequence GSGSGSGDITSLYKKAGST (1 letter amino acid code, SEQ ID NO: 11) derived in part from the Gateway recombination sequence.

Plasmid PS1767 encodes a fusion of NtermE[F64L]YFP172 and the FKBP binding part of FRAP, FRB (amino acids 2025-2114 of FRAP), connected by a linker sequence GSGSGSGDITSLYKKAGST (1 letter amino acid code, SEQ ID NO: 12) derived in part from the Gateway recombination sequence.

Plasmid PS1771 encodes a fusion FRB and CtermEYFP173, connected by a linker sequence DPAFLYKVVISGSGSGSG (1 letter amino acid code, SEQ ID NO: 13) derived in part from the Gateway recombination sequence.

Plasmid PS1768 encodes a fusion of FKBP and CtermEYFP173, connected by a linker sequence DPAFLYKVVISGSGSGSG (1 letter amino acid code, SEQ ID NO: 14) derived in part from the Gateway recombination sequence.

Construction of Plasmid PS1769.

Plasmid PS1769 encodes a fusion of NtermE[F64L]YFP172 and FKBP, connected by a linker sequence, under the control of a CMV promoter and with kanamycin and neomycin resistance as selectable marker in E. coli and mammalian cells, respectively.

Plasmid PS1769 was derived from plasmids PS1779 (entry clone) and PS1679 (destination vector). Plasmid PS1679 was derived from plasmids PS1672 and pEGFP-C1(Clontech). Plasmid PS1672 was derived from plasmid PS1641 described above.

Construction of Intermediate PS1672.

PS1641 was subjected to PCR with primers 2219 and 2222 (Table 2), and the ca 0.5 kb Nhe1-BamH1 fragment was ligated into pEGFP-C1 (Clontech) digested with Nhe1 and BamH1. This replaces NtermEGFP with NtermE[F64L]YFP172 followed by a linker sequence, which encodes in frame linker sequence Gly-Ser-Gly-Ser-Gly-Ser-Gly, and a unique EcoRV site just upstream of BamH1. This plasmid is called PS1672.

Construction of Destination Vector PS1679.

Plasmid PS1672 was converted into a Gateway compatible destination vector by cutting the DNA with EcoRV and ligating it with Gateway Cassette reading frame A, following the recommendations of the Gateway manufacturer (Invitrogen). This destination vector is called PS1679.

Construction of Gateway Entry Clone PS1779.

The coding sequence of FKBP (GenBank Acc no XM_{—}016660) was isolated from human cDNA using PCR and primers 2442 and 1272 (Table 2). The ca 0.4 kb product was transferred by a BP reaction into donor vector pDONR207, following the manufacturers recommendations (Invitrogen), to produce entry clone PS1779.

Finally, the expression vector PS1769 was produced by transferring FKBP from entry clone PS1779 with an LR reaction into destination vector PS1679 following the manufacturers recommendations (Invitrogen).

Construction of Plasmid PS1767.

Plasmid PS1767 encodes a fusion of NtermE[F64L]YFP172 and the FKBP binding part of FRAP, FRB (amino acids 2025-2114 of FRAP), connected by a linker sequence, under the control of a CMV promoter and with kanamycin and neomycin resistance as selectable marker in E. coli and mammalian cells, respectively.

Plasmid PS1767 was derived from plasmids PS1781 (entry clone) and PS1679 (destination vector). Plasmid PS1679 was constructed as described above.

Construction of Gateway Entry Clone PS1781.

The FKBP binding part of FRAP (amino acids 2025-2114, Gen Bank Acc no XM 001528) was isolated from human cDNA using PCR and primers 2444 and 1268 (Table 2). The ca 0.3 kb product was transferred by a BP reaction into donor vector pDONR207, following the manufacturers recommendations (Invitrogen), to produce entry clone PS1781.

Finally, the expression vector PS1767 was produced by transferring FRB from entry clone PS1781 with an LR reaction into destination vector PS1679 following the manufacturers recommendations (Invitrogen).

Construction of Plasmid PS1771.

Plasmid PS1771 encodes a fusion of the FKBP binding part of FRAP called FRB (amino acids 2025-2114 of FRAP) and the C-terminal of EYFP (FRB-CtermEYFP173), connected by a linker sequence, under the control of a CMV promoter and with zeocin resistance as selectable marker in E. coli and mammalian cells.

Plasmid PS1771 was derived from plasmids PS1782 (entry clone) and PS1688 (destination vector). Plasmid PS1688 was derived from plasmids PS1674 and PS609 described above. Plasmid PS1674 was derived from plasmid PS1638 described above.

Construction of Intermediate PS1674.

PS1638 was subjected to PCR with primers 2225 and 2132 (Table 2), and the ca 0.25 kb Nhe1-BamH1 fragment was ligated into PS609 digested with Nhe1 and BamH1. This replaces EGFP with EYFP(173-238) preceded by a linker sequence, which encodes in frame linker sequence Gly-Ser-Gly-Ser-Gly-Ser-Gly, and a unique EcoRV site just downstream of Nhe1. This plasmid is called PS1674.

Construction of Destination Vector PS1688.

Plasmid PS1674 was converted into a Gateway compatible destination vector by cutting the DNA with EcoRV and ligating it with Gateway Cassette reading frame A, following the recommendations of the Gateway manufacturer (Invitrogen). This destination vector is called PS1688.

Construction of Gateway Entry Clone PS1782.

The FKBP binding part of FRAP (GenBank Acc no XM_{—}001528, amino acids 2025-2114) was isolated from human cDNA using PCR and primers 2444 and 2445 (Table 2). The ca 0.3 kb product was transferred by a BP reaction into donor vector pDONR207, following the manufacturers recommendations (Invitrogen), to produce entry clone PS1782.

Finally, the expression vector PS1768 was produced by transferring FRB from entry clone PS1782 with an LR reaction into destination vector PS1688 following the manufacturers recommendations (Invitrogen).

Construction of Plasmid PS1768.

Plasmid PS1768 encodes a fusion of FKBP and EYFP(173-238) (FKBP-CtermEYFP173), under the control of a CMV promoter and with zeocin resistance as selectable marker in E. coli and mammalian cells.

Plasmid PS1768 was derived from plasmids PS1780 (entry clone) and PS1688 (destination vector). Plasmid PS1688 was constructed as described above.

Construction of Gateway Entry Clone PS1780.

The coding sequence of FKBP (GenBank Acc no XM_{—}016660) was isolated from human cDNA using PCR and primers 2442 and 2443 (Table 2). The ca 0.4 kb product was transferred by a BP reaction into donor vector pDONR207, following the manufacturers recommendations (Invitrogen), to produce entry clone PS1780.

Finally, the expression vector PS1768 was produced by transferring FKBP from entry clone PS1780 with an LR reaction into destination vector PS1688 following the manufacturers recommendations (Invitrogen).

### Example 11

### Construction of an Inducible Interaction System Using the GFP Complementation Method that Demonstrates Utility of the Method in Screening for Compounds that Inhibit Protein-Protein Interactions

The immunosuppressive compound rapamycin binds to FK506 binding protein (FKBP) and simultaneously to the large PI3Kinase homolog FRAP (also known as mTOR or RAFT), and thus serves as an heterodimeriser compound for these two proteins. To use rapamycin to induce heterodimers between proteins of interest, one of the proteins is fused to FKBP domains, and the other to a 90 amino acid portion of FRAP, termed FRB, that is sufficient for the binding the FKBP-rapamycin complex (Chen et al, PNAS 92, 4947 (1995)). In this example fusions of FRB and FKBP were made to complementary halves of split-EYFP (which included the F64L mutation in the EYFP(1-172) sequence (NtermE[F64L]YFP172)), so that the complementation reaction could be controlled by addition of rapamycin.

This example demonstrates that a model GFP complementation system using components which can be made to interact conditionally does respond as expected in a dose-dependent manner to the interaction stimulus. The example also provides information about the rate of fluorescence development for the E[F64L]YFP complementation system. Further it demonstrates that the system can be used to detect compounds that will block the interaction of proteins fused to the complementary halves of the E[F64L]YFP complementation system.

The following fusion constructs were made as described in Example 10:

NtermE[F64L]YFP172-FKBP=plasmid code PS1769

FRB-CtermEYFP173=PS1771

NtermE[F64L]YFP172-FRB=PS1767

FKBP-CtermEYFP173=PS1768

Probes were co-transfected in pairs into CHO-hIR cells (supra), PS1769 with PS1771 and PS1767 with PS1768, using the transfection agent FuGENE™ 6 (Boehringer Mannheim Corp, USA) according to the method recommended by the suppliers. Cells were cultured in growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Transfected cells were cultured in this medium, with the addition of two selection agents appropriate to the plasmids being used, being 1 mg/ml zeocin plus 0.5 mg/mlG418 sulphate. Cells were cultured at 37° C. in 100% humidity and conditions of normal atmospheric gases supplemented with 5% CO₂.

After 10 to 12 days culture in the continuous presence of the selection agents, the resulting cell lines were judged to be stably transfected. For fluorescence microscopy, aliquots of cells were transferred to Lab-Tek chambered cover glasses (Nalge Nunc International, Naperville USA) and allowed to adhere for at least 24 hours to reach about 80% confluence. Images were routinely collected using a Nikon Diaphot 300 inverted fluorescence microscope (Nikon Corp., Tokyo, Japan) using x20 (dry) and/or x40 (oil immersion) objectives and coupled to a Orca ER charged coupled device (CCD) camera (Hammamatsu Photonics K.K., Hammamatsu City, Japan). The cells are illuminated with a 100 W HBO arc lamp via a 470±20 nm excitation filter, a 510 nm dichroic mirror and a 515±15 nm emission filter for minimal image background. Image collection, subsequent measurement and analysis of fluorescence intensity were all controlled by IPLab Spectrum for Windows software (Scanalytics, Fairfax, Va. USA).

Cells were also grown for 16 hours from a seeding density of approximately 1.0×10⁵ cells per 400 μL in plastic 96-well plates (Polyfiltronics Packard 96-View Plate or Costar Black Plate, clear bottom; both types tissue culture treated) both for imaging purposes and for measurements of fluorescence intensity in fluorescence plate readers. Prior to experiments, the cells are cultured over night without selection agent(s) in HAM F-12 medium with glutamax, 100 μg penicillin-streptomycin mixture ml⁻¹ and 10% FBS. This medium has low auto fluorescence enabling fluorescence measurements on cells straight from the incubator. For endpoint measurements, cells in plates were routinely fixed with 4% formaldehyde in phosphate buffered saline (PBS)+10 μM Hoechst 22538 for 10 minutes, followed by 3 wash steps using PBS. The use of the nuclear dye Hoechst 22538 enables correction of the EYFP fluorescence signal from each well for cell density. Plates prepared in this way were measured on a Fluoroskan Ascent CF plate reader (Labsystems, Finland) equipped with appropriate filter sets (EYFP: excitation 485 nm, emission 527 nm; Hoechst 22538: excitation 355 nM, emission 460 nm).

Both cell lines CHO-hIR [PS1769+PS1771] and CHO-hIR [PS1767+PS1768] responded to rapamycin with a substantial increase in EYFP fluorescence after several hours incubation, as expected (FIG. 10). At the starting condition for these cells (t=0), fluorescence is barely visible in most cells, although it was noted that some cells (<5%) in the population had low, but appreciable, fluorescence before treatment (FIG. 10a). After 4 hours (FIG. 10b) many cells (approximately 40%) had developed significantly greater EYFP fluorescence throughout the cytoplasmic and nuclear compartments. After 16 hours (FIG. 10c) the response per cell had increased further and encompassed a larger proportion of the cell population (approximately 70%). Results were essentially identical for the second cell line CHO-hIR [PS1769+PS1771].

The graph in FIG. 11 shows the rate of development of cellular EYFP fluorescence following rapamycin treatment of the CHO-hIR [PSi 767+PS1768] line. Cells were treated in 96-well plates with 3 μM rapamycin and the fluorescence measured at various times. Treatment and measurements were made with the cells growing in HAM's medium+10% FBS, and fluorescence measurements were corrected for the background fluorescence from this medium. The graph demonstrates that the half-time for development of fluorescence is approximately 5 hours. The rate of development of fluorescence includes time for interaction between FKBP and FRB mediated by the dimeriser rapamycin, plus the time for annealing of the EYFP moieties, and the (presumably much longer) time needed for maturation of the fluorophore within the successfully annealed EYFP protein.

FIG. 12 is a response curve to different rapamycin doses for the CHO-hIR [PS1769+PS1771] cell line. Cells were cultured in 96-well plates, treated with various rapamycin doses for 16 hours, then fixed and stained with Hoechst prior to determination of EYFP fluorescence/cell (arbitrary units) on the Ascent plate reader. Values are corrected for PBS background as well as cell number. The cell line shows approximately a 3-fold increase in the EYFP intensity/cell over the dose range of rapamycin used in this experiment.

One way to increase the dynamic range of the response, and to decrease the inherent EYFP background signal from these cell lines, is to remove the fraction of cells that are EYFP bright prior to rapamycin stimulation. This is easily accomplished through fluorescence activated cell sorting (FACS) methods. Each of the cell lines were sorted by this method into 3 groups: (i) most green group (ii) medium to low-green group and (iii) black group. The ‘most green’ was discarded in each case, while the other 2 groups were cultured for further use. FIG. 13 (a) and FIG. 13 (b) show the improved response to 100 nM rapamycin of cell line CHO-hIR [PS1767+PS1768] after the sorting procedure.

FIGS. 14 (a) and (b) show the response of the ‘medium to low-green’ and ‘black’ FACS groups (respectively) derived from the CHO-hIR [PS1767+PS1768] parent line. Dose response to rapamycin was measured after 7 hours (a) and 30 hours (b) for each cell line. Values for fluorescence have been corrected for plate & medium background. Increase in EYFP fluorescence is better than 20-fold the unstimulated value in each case. Unexpectedly, the absolute fluorescence signal does not appear to change significantly between 7 and 30 hours, although the cells are still alive during this period. Furthermore, the dose-response curves at 7 and 30 hours for each cell line are very closely similar, with an EC₅₀ of approximately 0.25 μM in the ‘medium to low-green’ group, and 0.1 μM in the ‘black’ group. This data suggest that once the dimerisation has occurred, the EYFP complements are stable within the cells for longer than 30 hours. The ‘medium to low-green’ group has a greater overall response range, reaching intensities of greater than 3-fold that of the black group at the highest rapamycin concentration. Both FACS groups have significantly lower pre-stimulation fluorescence intensities compared to the parent (non-FACS'd) lines.

FIGS. 15 (a) and (b) show dose-response competition curves for FK506 versus 100 nM rapamycin in two of the FACS'd lines, CHO-hIR [PS1768+PS17671 ‘mid to low-green’ group (FIG. 15 (a)) and CHO-hIR [PS1769+PS1771] ‘black’ group (FIG. 15 (b)). EC₅₀ values in both cases are approximately 1.2 μM FK506. The cells were incubated overnight (16 hours) with mixtures of the two compounds, then fixed and stained with Hoechst prior to determination of EYFP fluorescence/cell on an Ascent plate reader. Plate and solution backgrounds have been subtracted; the dashed lines on each graph indicate the prestimulated fluorescence levels for each cell line in these experiments. These results indicate that the GFP complementation method employing fusions to NtermE[F64L]YFP172 and CtermEYFP173 may be used successfully to screen for compounds that interfere with a conditional interaction between two protein components.

### Example 12

### Construction of PS1736, PS1758, PS1706, PS1810, PS1809, and PS1827

Plasmid PS1736 encodes a fusion of F64L,NY172-NZ and H2B, connected by a linker sequence GSGSGSGDITSLYKKAGST (1 letter amino acid code, SEQ ID NO: 74) derived in part from the Gateway recombination sequence.

Plasmid PS1758 encodes a fusion of beta-Catenin and CZ-CY173, connected by a linker sequence DPAFLYKWISGSGSGSG (1 letter amino acid code, SEQ ID NO: 69) derived in part from the Gateway recombination sequence.

Plasmid PS1706 encodes a fusion of F64L,NY172 (=NtermE[F64L]YFP172) and beta-Catenin, connected by a linker sequence GSGSGSGDITSLYKKAGST (1 letter amino acid code, SEQ ID NO: 70) derived in part from the Gateway recombination sequence.

Plasmid PS1810 encodes a fusion of H2B and TCF4(1-70) and CY173 (CtermEYFP173), where H2B and TCF4(1-70) are connected by a linker sequence DITSLYKKAGST (SEQ ID NO: 75) derived in part from the Gateway recombination sequence, and where TCF4(1-70) and CY173 (CtermEYFP173) are connected by a linker sequence DPAFLYKWISGSGSG (1 letter amino acid code, SEQ ID NO: 71) derived in part from the Gateway recombination sequence.

Plasmid PS1809 encodes a fusion of F64L,NY172 (NtermE[F64L]YFP172) and FKBP and beta-Catenin, where F64L,NY172 (NtermE[F64L]YFP172) and FKBP are connected by a linker sequence GSGSGSGDL (1 letter amino acid code, SEQ ID NO: 72) and where FKBP and beta-Catenin are connected by a linker sequence GTGTGTGDITSLYKKAGST (SEQ ID NO: 76) derived in part from the Gateway recombination sequence.

Plasmid PS1827 encodes a fusion of H2B and the FKBP binding part of FRAP (amino acids 2025-2114) and CY173 (CtermEYFP173), where H2B and the FKBP binding part of FRAP (amino acids 2025-2114) are connected by a linker sequence DPAFLYKWISGTGTGTG (1 letter amino acid code, SEQ ID NO: 73) derived in part from the Gateway recombination sequence, and where the FKBP binding part of FRAP (amino acids 2025-2114) and CY173 (CtermEYFP173) are connected by a linker sequence LPSGSGSGSG (SEQ ID NO: 77).

Construction of Plasmid PS1736.

Plasmid PS1736 encodes a fusion of F64L,NY172-NZ and H2B, connected by a linker sequence, under the control of a CMV promoter and with kanamycin and neomycin resistance as selectable marker in E. coli and mammalian cells, respectively.

Plasmid PS1736 was derived from plasmids PS1667 (entry clone) and PS1695 (destination vector). Plasmid PS1695 was derived from plasmid PS1673. Plasmid PS1673 was derived from plasmids PS1641 described above and pEGFP-C1(Clontech). Plasmid PS1672 was derived from plasmid PS1641 described above.

Construction of Intermediate PS1673.

PS1641 was subjected to PCR with primers 2219 and 2387 (table 2), and the ca 0.5 kb Nhe1-BamH1 fragment was ligated into pEGFP-C1 (Clontech) digested with Nhe1 and BamH1. This replaces EGFP with F64L,EYFP(1-172) followed by a linker sequence, which encodes in frame linker sequence GGTGSG-NZ-GSGSGSG, and a unique EcoRV site just upstream of BamHI. This plasmid is called PS1673.

Construction of Destination Vector PS1695.

Plasmid PS1673 was converted into a Gateway compatible destination vector by cutting the DNA with EcoRV and ligating it with Gateway Cassette reading frame A, following the recommendations of the Gateway manufacturer (Invitrogen). This destination vector is called PS1695.

Construction of Gateway Entry Clone PS1667.

The coding sequence of H2B (GenBank Acc no NM_{—}003518) was isolated from human cDNA using PCR and primers 2367 and 2368 (Table 2). The ca 0.4 kb product was transferred by a BP reaction into donor vector pDONR207, following the manufacturers recommendations (Invitrogen), to produce entry clone PS1667

Finally, the expression vector PS1736 was produced by transferring H2B from entry clone PS1667 with an LR reaction into destination vector PS1695 following the manufacturers

Construction of Plasmid PS1758.

Plasmid PS1758 encodes a fusion of beta-Catenin and CZ-CY173, connected by a linker sequence, under the control of a CMV promoter and with zeocin resistance as selectable marker in E. coli and mammalian cells.

Plasmid PS1771 was derived from plasmids PS1762 (entry clone) and PS1696 (destination vector). Plasmid PS1696 was derived from plasmid PS1726. Plasmid PS1726 was derived from plasmids PS1638 and PS609 described above.

Construction of Intermediate PS1726.

PS1638 was subjected to PCR with primers 2388 and 2132 (table 2), and the ca 0.35 kb Nhe1-BamH1 fragment was ligated into PS609 digested with Nhe1 and BamH1. This replaces EGFP with EYFP(173-238) preceded by a linker sequence, which encodes in frame linker sequence GSGSGSG-zip, and a unique EcoRV site just downstream of Nhe1. This plasmid is called PS1726.

Construction of Destination Vector PS1696. Plasmid PS1726 was converted into a Gateway compatible destination vector by cutting the DNA with EcoRV and ligating it with Gateway Cassette reading frame A, following the recommendations of the Gateway manufacturer (Invitrogen). This destination vector is called PS1696.

Construction of Gateway Entry Clone PS1762.

The coding sequence of beta-Catenin (GenBank Acc no X87838, was isolated from human cDNA using PCR and primers 2359 and 2439 (Table 2). The ca 2.3 kb product was transferred by a BP reaction into donor vector pDONR207, following the manufacturers recommendations (Invitrogen), to produce entry clone PS1762.

Finally, the expression vector PS1758 was produced by transferring the coding sequence of beta-Catenin from entry clone PS1762 with an LR reaction into destination vector PS1696 following the manufacturers recommendations (Invitrogen).

Construction of Plasmid PS1706.

Plasmid PS1706 encodes a fusion of F64L,NY172 and beta-Catenin, connected by a linker sequence, under the control of a CMV promoter and with kanamycin and neomycin resistance as selectable marker in E. coli and mammalian cells, respectively.

Plasmid PS1706 was derived from plasmids PS1664 (entry clone) and PS1679 (destination vector). Plasmid PS1679 is described above.

Construction of Gateway Entry Clone PS1664.

The coding sequence of beta-Catenin (GenBank Acc no X87838) was isolated from human cDNA using PCR and primers 2359 and 2360 (table 2). The ca 2.3 kb product was transferred by a BP reaction into donor vector pDONR207, following the manufacturers recommendations (Invitrogen), to produce entry clone PS1664.

Finally, the expression vector PS1706 was produced by transferring beta-Catenin from entry clone PS1664 with an LR reaction into destination vector PS1679 following the manufacturers recommendations (Invitrogen).

Construction of Plasmid PS1810.

Plasmid PS1810 encodes a fusion of H2B-TCF4(1-70) and CY173, connected by a linker sequence, under the control of a CMV promoter and with zeocin resistance as selectable marker in E. coli and mammalian cells.

Plasmid PS1810 was derived from plasmids PS1669 (entry clone) and PS1806 (destination vector). Plasmid PS1806 was derived from plasmid PS1790. Plasmid PS1790 was derived from plasmid PS1674 described above.

Construction of Intermediate PS1790.

The coding sequence of H2B (GenBank Acc no NM_{—}003518) was isolated from human cDNA with primers 2389 and 2390 (Table 2), and the ca 0.4 kb Nhe1-EcoRV fragment was ligated into PS1674 digested with Nhe1 and EcoRV. This places H2B upstream of EYFP(173-238), which is preceded by a linker sequence, which encodes in frame linker sequence GSGSGSG-zip, and a unique EcoRV site between H2B and the linker-EYFP(173-238). This plasmid is called PS1790.

Construction of Destination Vector PS1806.

Plasmid PS1790 was converted into a Gateway compatible destination vector by cutting the DNA with EcoRV and ligating it with Gateway Cassette reading frame A, following the recommendations of the Gateway manufacturer (Invitrogen). This destination vector is called PS1806.

Construction of Gateway Entry Clone PS1669.

The coding sequence of the N-terminal 70 amino acids of TCF4 (GenBank Acc no Y11306), was isolated from human cDNA using PCR and primers 2364 and 2366 described below. The ca 0.25 kb product was transferred by a BP reaction into donor vector pDONR207, following the manufacturers recommendations (Invitrogen), to produce entry clone PS1669.

Finally, the expression vector PS1810 was produced by transferring the coding sequence of TCF4(1-70) from entry clone PS1669 with an LR reaction into destination vector PS1806 following the manufacturers recommendations (Invitrogen).

Construction of Plasmid PS1809.

Plasmid PS1809 encodes a fusion of F64L,NY172 and FKBP-betaCatenin, connected by a linker sequence, under the control of a CMV promoter and with kanamycin and neomycin resistance as selectable marker in E. coli and mammalian cells, respectively.

Plasmid PS1809 was derived from plasmids PS1664 (entry clone) described above and PS1805 (destination vector). Plasmid PS1805 was derived from plasmid PS1789. Plasmid PS1789 was derived from plasmids PS1672 described above and PS1768 described above.

Construction of Intermediate PS1789.

PS1768 was subjected to PCR with primers 2462 and 2463, and the ca 0.35 kb fragment was digested with Pvu2 and BamHI and ligated into PS1672 digested with EcoRV and BamH1. This produces a fusion of F64L,NY172 and FKBP connected by a GSGSGSGDL linker and with a GTGTGTG linker sequence behind FKBP and a unique EcoRV site just downstream of the GT3 linker. This plasmid is called PS1789.

Construction of Destination Vector PS1805.

Plasmid PS1789 was converted into a Gateway compatible destination vector by cutting the DNA with EcoRV and ligating it with Gateway Cassette reading frame A, following the recommendations of the Gateway manufacturer (Invitrogen). This destination vector is called PS1805.

Finally, the expression vector PS1809 was produced by transferring beta-Catenin from entry clone PS1664 with an LR reaction into destination vector PS1805 following the manufacturers recommendations (Invitrogen).

Construction of Plasmid PS1827.

Plasmid PS1827 encodes a fusion of H2B and the FKBP binding part of FRAP (amino acids 2025-2114) and CY173, connected by a linker sequence, under the control of a CMV promoter and with zeocin resistance as selectable marker in E. coli and mammalian cells.

Plasmid PS1827 was derived from plasmids PS1732 (entry clone) and PS1826 (destination vector). Plasmid PS1826 was derived from plasmid PS1788. Plasmid PS1788 was derived from plasmids PS1674 and PS1767 described above.

Construction of Intermediate PS1788.

PS1767 was subjected to PCR with primers 2464 and 2465, and the ca 0.35 kb product was digested with Nhe1-Sma1 and ligated into PS1674 digested with Nhe1 and EcoRV. This produces a fusion of FRAP(2025-2114) and EYFP(173-238) connected by a linker sequence, GSGSGSG, and with a GTGTGTG linker sequence in front of FRAP, and a unique EcoRV site just upstream of the GTGTGTG linker. This plasmid is called PS1788.

Construction of Destination Vector PS1826.

Plasmid PS1788 was converted into a Gateway compatible destination vector by cutting the DNA with EcoRV and ligating it with Gateway Cassette reading frame A, following the recommendations of the Gateway manufacturer (Invitrogen). This destination vector is called PS1826.

Construction of Gateway Entry Clone PS1732.

The coding sequence of H2B (GenBank Acc no NM_{—}003518) was isolated from human cDNA using PCR and primers 2367 and 2407 (Table 2). The ca 0.4 kb product was transferred by a BP reaction into donor vector pDONR207, following the manufacturers recommendations (Invitrogen), to produce entry clone PS1732

Finally, the expression vector PS1827 was produced by transferring H2B from entry clone PS1732 with an LR reaction into destination vector PS1826 following the manufacturers recommendations (Invitrogen).

### Example 13

### Assay for Screening in Live Cells for Compounds that Modify the Translocation Behaviours of One or Both Members of a Pair of Target Proteins and/or the Interaction Between Those Proteins

The present example shows the construction of a protein translocation assay using the GFP complementation method that demonstrates utility of the method in screening in live cells for compounds that modify the translocation behaviours of one or both members of a pair of target proteins and/or the interaction between those proteins (Translocation and Interaction Dependent Complementation, TIDC).

This example demonstrates that GFP complementation systems can be designed to report on the degree of interaction between one protein and a partner protein within living cells, specifically where such an interaction can only take place after translocation of one or both proteins has occurred that serves to bring both components together in one compartment or location within the cell. A fluorescence signal is obtained that corresponds with the degree of interaction that occurs between the partner proteins, usually following a stimulus that induces the appropriate translocation. Such an assay system can be used to screen for compounds or treatments that will modify either the translocation process(es) involved or the interaction between the target proteins used in construction of the complementation probes.

β-catenin is a multifunctional protein that plays a key role in the development and progression of certain human cancers, most notably cancer of the colon. In a non-dividing colonic epithelial cell β-catenin is found in complexes with cell adhesion molecules at the plasma membrane, and also in complex with a number of proteins (Axin, Adenomatous polyposis coli protein, or APC, and the protein kinase GSK3β) that together control its targeted destruction by the cellular ubiquitination system. β-catenin also functions as an activator of transcription, for which function it needs to translocate to the nucleus and dimerise with other pro-transcriptional co-factors, the most significant of which in colonic cells is TCF4. The level of β-catenin in non-dividing cells is kept low by ubiquitination and subsequent destruction by proteosomes, and little or no β-catenin is found in the nucleus, and thus little or no β-catenin-induced gene transcription occurs. In cancerous and certain pre-cancerous colon epithelial cells, mutations in either β-catenin itself or in one of its regulatory proteins lead to accumulation of the protein in the cytoplasm, with subsequent translocation to the nucleus and constitutive activation of transcription from genes controlled by β-catenin:TCF4 factors. It is continuous expression of these genes that leads to the cancerous phenotype in such cells.

In this example, complementary halves of GFP are fused separately to β-catenin and also to histone H2B coupled to the β-catenin-binding fragment of the transcriptional co-activator TCF4. Both constructs are then co-transfected and expressed within mammalian cells. The β-catenin construct is expected to be localised entirely in the cell cytoplasm at a low level, its concentration effectively controlled by the activity of the protein kinase GSK3β. The H2B-TCF4 construct will be localised in the nuclei of cells by the highly basic histone moeity. Hence, the two components should not meet one another under normal growth conditions. However, when GSK3β is inhibited by lithium ions, the cytoplasmic level of the β-catenin construct is expected to increase in the cytoplasm, and to translocate in significant fashion to the nucleus. When the constructs find themselves mixed together in the nucleus as a result of β-catenin translocation, the natural interaction between the two proteins (β-catenin and TCF4) will bring the complementary GFP halves into close apposition, whereupon the annealing process should be initiated that will ultimately lead to development of fully fluorescent GFP molecules. The development of nuclear fluorescence in lithium-treated cells therefore effectively reports on the combined result of translocation of β-catenin to the nucleus and the subsequent interaction between β-catenin and the β-catenin binding domain of TCF4.

The following fusion constructs were made (see Example 12):

NtermE[F64L]YFP172-β-catenin=plasmid code ps1706

H2B-TCF4(frag)-CtermEYFP173=ps1810

Plasmids ps1706 with ps1810 were co-transfected into chinese hamster ovary cells (CHO) using the transfection agent FuGENE™ 6 (Boehringer Mannheim Corp, USA) according to the method recommended by the suppliers. Cells were cultured in growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Transfected cells were cultured in this medium, with the addition of two selection agents appropriate to the plasmids being used, being 1 mg/ml zeocin plus 0.5 mg/mlG418 sulphate. Cells were cultured at 37° C. in 100% humidity and conditions of normal atmospheric gases supplemented with 5% CO₂.

After 10 to 12 days culture in the continuous presence of the selection agents, the resulting cell lines were judged to be stably transfected. For fluorescence microscopy, aliquots of cells were transferred to Lab-Tek chambered cover glasses (Nalge Nunc International, Naperville USA) and allowed to adhere for at least 24 hours to reach about 80% confluence. Images were collected using a Zeiss LSM410 inverted confocal fluorescence microscope (Zeiss, Jena, Germany) using x40 (oil immersion) objective. 488 nm laser light was used for excitation of GFP, emissions filtered via a FT510 dichroic and 510-525 nm bandpass filter.

After treatments, but prior to microscopy, cells were routinely fixed with 4% formaldehyde in phosphate buffered saline (PBS) for 10 minutes, followed by 3 wash steps using PBS.

FIG. 18 (A) shows the level of fluorescence in stable CHO[ps1706+ps1810] prior to any treatment of the cells. The level of fluorescence is very weak in most cells and may be due entirely to cellular autofluorescence from flavanoids such as FAD (the emision filter used typically allows a fraction of FAD autofluorescence to pass). Occasional cells (top right corner of FIG. 18 (A)) show a weak GFP fluorescence in nuclear and cytoplasmic compartments, apparently the result of spontaneous complementation. Most of these cells are found clustered, possibly indicating a clonal behaviour that may reflect incorrect processing of the introduced components, such that they are brought together into the same compartment during expression (or possibly cell division) where complementation can occur. However, when treated for 16 hours with 5 mM LiCl, the number of cells containing fluorescent GFP increases greatly, and many of these have the GFP fluorescence confined exclusively to their nuclei consistent with the distribution of GFP complementation expected from this pair of fusion constructs (FIG. 18 (B)).

These results indicate that the GFP complementation method employing fusions to NtermE[F64L]YFP172 and CtermEYFP173 of protein components that normally reside in separated cellular compartments or locations, can be used to monitor the protein-protein interaction between those components that result from translocation processes bringing both components together within one compartment or at one shared location. A simple intensity or image-based fluorescence assay based on this principle may be used to screen for compounds that affect either the translocation processes relevant to specific cellular components or the interactions between such protein components in living mammalian cells. Used in combination with a similar assay designed to report solely on the translocation processes involved (e.g. an assay similar to that described in Example 14 or Example 16), it will be possible to define whether any compound is specifically targeting translocation or interaction.

### Example 14

### Protein Translocation Assay Using GFP Complementation

This method that demonstrates utility of the method in screening in live cells for compounds that modify the translocation behaviour of a target protein (Translocation Dependent Complementation, TDCzip).

This example demonstrates that GFP complementation systems can be designed to report specifically on the translocation behaviour of a target protein within living cells. A fluorescence signal is obtained that corresponds directly with the degree of translocation that occurs. Such an assay system can be used to screen for compounds or treatments that will modify the translocation behaviour of the target protein.

β-catenin is a multifunctional protein that plays a key role in the development and progression of certain human cancers, most notably cancer of the colon. In a non-dividing colonic epithelial cell β-catenin is found in complexes with cell adhesion molecules at the plasma membrane, and also in complex with a number of proteins (Axin, Adenomatous polyposis coli protein, or APC, and the protein kinase GSK3β) that together control its targeted destruction by the cellular ubiquitination system. β-catenin also functions as an activator of transcription, for which function it needs to translocate to the nucleus and dimerise with other pro-transcriptional co-factors, the most significant of which in colonic cells is TCF4. The level of β-catenin in non-dividing cells is kept low by ubiquitination and subsequent destruction by proteosomes, and little or no β-catenin is found in the nucleus, and thus little or no β-catenin-induced gene transcription occurs. In cancerous and certain pre-cancerous colon epithelial cells, mutations in either β-catenin itself or in one of its regulatory proteins lead to accumulation of the protein in the cytoplasm, with subsequent translocation to the nucleus and constitutive activation of transcription from genes controlled by β-catenin:TCF4 factors. It is continuous expression of these genes that leads to the cancerous phenotype in such cells.

In this example, complementary halves of GFP are fused separately to β-catenin and to histone H2B and co-expressed within cells. The β-catenin construct is expected to be localised entirely in the cell cytoplasm at a low level, its concentration effectively controlled by the activity of the protein kinase GSK3β. The H2B construct will be localised in the nuclei of cells by the highly basic histone moeity. Hence, the two components should not meet one another under normal growth conditions. However, when GSK3β is inhibited by lithium ions, the cytoplasmic level of the β-catenin construct is expected to increase in the cytoplasm, and to translocate in significant fashion to the nucleus. Both constructs have been engineered to contain a leucine zipper domain. When the constructs find themselves mixed together in the nucleus as a result of β-catenin translocation, the leucine zippers will dimerise bringing the complementary GFP halves into close apposition, whereupon the annealing process is initiated that ultimately leads to development of fully fluorescent GFP molecules. The development of nuclear fluorescence in lithium-treated cells therefore effectively reports on the translocation of β-catenin to the nucleus.

The following fusion constructs were made (see Example 12):

NtermE[F64L]YFP172-Zip-H2B=plasmid code ps1736

β-catenin-Zip-NternE[F64L]YFP172=ps1758

Plasmids ps1736 with ps1758 were co-transfected into chinese hamster ovary cells (CHO) using the transfection agent FuGENE™ 6 (Boehringer Mannheim Corp, USA) according to the method recommended by the suppliers. Cells were cultured in growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Transfected cells were cultured in this medium, with the addition of two selection agents appropriate to the plasmids being used, being 1 mg/ml zeocin plus 0.5 mg/mlG418 sulphate. Cells were cultured at 37° C. in 100% humidity and conditions of normal atmospheric gases supplemented with 5% CO₂.

After 10 to 12 days culture in the continuous presence of the selection agents, the resulting cell lines were judged to be stably transfected. For fluorescence microscopy, aliquots of cells were transferred to Lab-Tek chambered cover glasses (Nalge Nunc International, Naperville USA) and allowed to adhere for at least 24 hours to reach about 80% confluence. Images were routinely collected using a Nikon Diaphot 300 inverted fluorescence microscope (Nikon Corp., Tokyo, Japan) using x20 (dry) and/or x40 (oil immersion) objectives and coupled to a Orca ER charged coupled device (CCD) camera (Hammamatsu Photonics K.K., Hammamatsu City, Japan). The cells are illuminated with a 100 W HBO arc lamp via a 470±20 nm excitation filter, a 510 nm dichroic mirror and a 515±15 nm emission filter for minimal image background. Image collection, subsequent measurement and analysis of fluorescence intensity were all controlled by IPLab Spectrum for Windows software (Scanalytics, Fairfax, Va. USA).

After treatments, cells were routinely fixed with 4% formaldehyde in phosphate buffered saline (PBS) for 10 minutes, followed by 3 wash steps using PBS.

FIG. 19 (A) shows the level of fluorescence in stable CHO[ps1736+ps1758] prior to any treatment of the cells. The level of fluorescence is very weak and may be due entirely to cellular autofluorescence from flavanoids such as FAD (the emision filter used typically allows a fraction of FAD autofluorescence to pass). The distribution of this fluorescence is also consistent with FAD autofluorescence, it being principally in the cytoplasm, with a somewhat punctate appearance. When treated for 16 hours with 5 mM LiCl, a fraction of these cells display GFP-bright nuclei, consistent with the distribution of GFP complementation expected from this pair of fusion constructs (FIG. 19 (B)).

These results indicate that the GFP complementation method employing fusions to NtermE(F64L]YFP172 and CtermEYFP173 of protein components that normally reside in separated cellular compartments or locations, can be used to monitor those translocation processes that result in both components coming together within one compartment or at one shared location. A simple intensity or image-based fluorescence assay based on this principle may be used to screen for compounds that affect the translocation processes relevant to specific cellular components in living mammalian cells.

### Example 15

### Exploitation of the Inducible FRBP12:FRB Interaction

As illustrated in Example 14, some assays will experience a sink effect: the problem is that the natural shuttling of any naturally translocating protein is exposing the anchored zipper construct to a constant supply of binding partner resulting in irreversible binding and premature fluorophore formation. The same problem can be encountered in TIDC systems that depend on a constitutive natural interaction like the Interaction between beta-catenin and TCF-4 (example 14).

The FKBP12:FRB system, is a convenient way of associating e.g. the nuclear fraction of a translocating protein fused to a complementation partner A to an anchor fused to complementation partner B at any given time.

The fusions that appear most appropriate based on the co-crystal structure of FKBP12:rapamycin:FRB (FIG. 17) is to fuse the both split EYFP F64L fragments to the C-termini of FKBP12 and FRB because they are close to each other. However, as we generally favor fusing the N-terminal split fragment to the N-terminus of the fusion partner and visa versa, this orientation was chosen in Example 16. This is in part due to our experience that distance of at least 40 Å between the termini does not limit the complementation (GFP is about 40 Å long). Using FKBP12 and FRB we will need 10-15 residue linkers on both sides of FKBP12 and FRB. The linker length will be between 1.5 Å (α-helix) and 3.5 Å (β-sheet) per residue (see FIG. 17).

### Example 16

### Protein Translocation Assay with Inducible Interaction Partners

This example describes the construction of a protein translocation assay using an inducible GFP complementation method that demonstrates utility of the method in screening in live cells for compounds that modify the translocation behaviour of a target protein (Translocation Dependent Complementation with Rapamycin, TDCrap).

This example demonstrates that an inducible GFP complementation systems can be designed to report specifically on the translocation behaviour of a target protein within living cells with an improved signal to background ratio. Following induction of the translocation process, complementation is stimulated by bringing together the interacting proteins and a fluorescence signal is obtained that corresponds directly with the degree of translocation that occurs. Such an assay system can be used to screen for compounds or treatments that will modify the translocation behavior of the target protein.

An additional linker sequence has been added in each because we no longer have the tightly associated zipper peptides involved. The Gateway recombination sequences are about 10 residues which should be enough between the inserted fusion proteins and FKBP12/FRB. The connections to the split fragments should be 12-20 residues.

β-catenin is a multifunctional protein that plays a key role in the development and progression of certain human cancers, most notably cancer of the colon. In a non-dividing colonic epithelial cell β-catenin is found in complexes with cell adhesion molecules at the plasma membrane, and also in complex with a number of proteins (Axin, Adenomatous polyposis coli protein, or APC, and the protein kinase GSK3β) that together control its targeted destruction by the cellular ubiquitination system. β-catenin also functions as an activator of transcription, for which function it needs to translocate to the nucleus and dimerise with other pro-transcriptional co-factors, the most significant of which in colonic cells is TCF4. The level of β-catenin in non-dividing cells is kept low by ubiquitination and subsequent destruction by proteosomes, and little or no β-catenin is found in the nucleus, and thus little or no β-catenin-induced gene transcription occurs. In cancerous and certain pre-cancerous colon epithelial cells, mutations in either β-catenin itself or in one of its regulatory proteins lead to accumulation of the protein in the cytoplasm, with subsequent translocation to the nucleus and constitutive activation of transcription from genes controlled by β-catenin:TCF4 factors. It is continuous expression of these genes that leads to the cancerous phenotype in such cells.

In this example, complementary halves of GFP are fused separately to β-catenin and to histone H2B and co-expressed within cells. The β-catenin construct is expected to be localised entirely in the cell cytoplasm at a low level, its concentration effectively controlled by the activity of the protein kinase GSK3β. The H2B construct will be localised in the nuclei of cells by the highly basic histone moeity. Hence, the two components should not meet one another under normal growth conditions. However, when GSK3β is inhibited by lithium ions, the cytoplasmic level of the β-catenin construct is expected to Increase in the cytoplasm, and to translocate in significant fashion to the nucleus. Both constructs have also been engineered to contain inducible interaction domains. The β-catenin construct includes FK506 binding protein (FKBP). The H2B construct includes FRB, which is the 93 amino acid portion of FRAP, the large PI3Kinase homolog FRAP (also known as mTOR or RAFT). The immunosuppressive compound rapamycin binds to FKBP and simultaneously to FRB, and thus serves as an heterodimeriser compound for these two proteins. The complementation reaction between the separate halves of GFP that are included in these two constructs can therefore be induced by addition of rapamycin, but only if the protein constructs are able to meet each other at the same cellular location. Because FKBP and FRB do not interact in the absense of rapamycin, the “sink effect” is greatly reduced or eliminated.

When the constructs find themselves mixed together in the nucleus as a result of β-catenin translocation, addition of rapamycin brings the complementary GFP halves into close apposition via the inducible FKBP:FRB interaction, whereupon the annealing process should be initiated that will ultimately lead to development of fully fluorescent GFP molecules. In this example, the development of nuclear GFP fluorescence that results after complementation is induced by rapamycin therefore reports on the degree of translocation into the nucleus of the β-catenin construct at the time of rapamycin addition.

The following fusion constructs were made (see Example 12):

NtermE[F64L]YFP172-FKBP-beta-Catenin=plasmid code ps1809

H2B-FRAP(2024-2114)-CtermEYFP173=ps1827.

Plasmids ps1809 with ps1827 were co-transfected into chinese hamster ovary cells (CHO) using the transfection agent FuGENE™ 6 (Boehringer Mannheim Corp, USA) according to the method recommended by the suppliers. Cells were cultured in growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark)). Transfected cells were cultured in this medium, with the addition of two selection agents appropriate to the plasmids being used, being 1 mg/ml zeocin plus 0.5 mg/mlG418 sulphate. Cells were cultured at 37° C. in 100% humidity and conditions of normal atmospheric gases supplemented with 5% CO₂.

After 10 to 12 days culture in the continuous presence of the selection agents, the resulting cell lines were judged to be stably transfected. For fluorescence microscopy, aliquots of cells were transferred to Lab-Tek chambered cover glasses (Nalge Nunc International, Naperville USA) and allowed to adhere for at least 24 hours to reach about 80% confluence. All treatments were carried out in full HAM's medium+10% FBS. Images were collected using a Zeiss LSM410 inverted confocal fluorescence microscope (Zeiss, Jena, Germany) using x40 (oil immersion) objective. 488 nm laser light was used for excitation of GFP, emissions filtered via a FT510 dichroic and 510-525 nm bandpass filter.

After treatments, but prior to microscopy, cells were routinely fixed with 4% formaldehyde in phosphate buffered saline (PBS) for 10 minutes, followed by 3 wash steps using PBS.

FIG. 20 shows a montage of CHO cells expressing ps1809 with ps1827, imaged from 4 separate treatments. The top row (FIG. 20 a-d) are images of GFP fluorescence. The GFP is localised to nuclei only of these cells, as demonstrated in the lower row of images (FIG. 20 e-h) where transmitted-light images have been overlayed with their corresponding GFP fluorescence. In FIGS. 20 c & d, the cells were treated for 16 hours with 5 mM lithium chloride to inhibit the kinase GSK3β, directly after transfer to the Lab-Tek chambered cover glasses; cells in FIGS. 20 a & b received no lithium during this time. Cells in FIGS. 20 b & d were then treated with 3 μM rapamycin (in ethanol stock, final concentration of ethanol was 0.6% v/v), whilst those in FIGS. 20 a & c received only 0.6% ethanol. Both treatments were continued for a further 6 hours before the cells were fixed and imaged.

Without lithium treatment (FIGS. 20 a & b) the level of fluorescence is very weak in most cells and may be due entirely to cellular autofluorescence from flavanoids such as FAD (the emision filter used typically allows a fraction of FAD autofluorescence to pass).

Occasional cells show a weak GFP fluorescence in nuclei, apparently the result of spontaneous complementation that may accompany nuclear membrane breakdown at cell division. When treated for 16 hours with 5 mM LiCl, the number of cells containing fluorescent GFP increases greatly, and many of these have the GFP fluorescence confined exclusively to their nuclei consistent with the distribution of GFP complementation expected from this pair of fusion constructs (FIGS. 20 c & d). The level of nuclear fluorescence in Li-treated cells increases substantially with rapamycin treatment (FIG. 20 d), the dimeriser increasing both the number of cells with bright nuclei, and the average intensity of GFP in each responding cell.

In a screening context, the present example show that in the untreated cells (a) no spontaneous complementation takes place (compared to FIG. 19 (A)), thereby avoiding the “sink” effect).

These results indicate that the GFP complementation method can be configured to monitor the translocation of a protein component within living cells, and that the efficiency of the method, as measured by signal to background ratio, can be significantly improved by inclusion of an inducible protein-protein interaction linkage such as provided by FKBP:FRB in the presence of rapamycin. Such a configuration of the system could be used to construct high throughput intensity-based fluorescence assays to identify compounds (or other influences) that act to modulate translocations of cellular components.

### FIGURE LEGENDS

FIG. 1

General structures of the fusion protein coding sequences.

FIG. 2

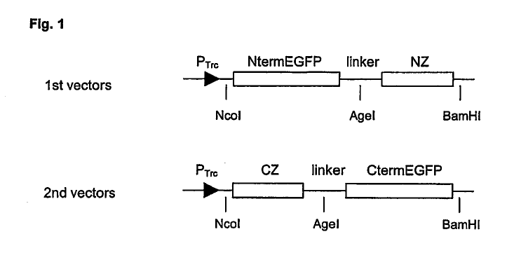

16 bit images of fluorescent CHO-hIR cells co-transfected with NtermEGFP-NZ and CZ-CtermEGFP expression vectors or transfected with pEGFP-C1 were taken and scaled individually to visualise the cells and the fluorescence distribution within them. Because of the pixel intensity scaling, the relative fluorescence levels cannot be compared among the images. The splitting sites are either between residues 157/158 (top row, plasmids PS1557 and PS1559) or between residues 172/173 (middle row, plasmids PS1558 and PS1560). The EGFP expression vector pEGFP-C1 was transfected into the cells in the bottom row. The images were taken 1 day (left column), 2 days (middle column), or 10 days (right column) after transfection. The images of the cells are representative of the cells that expressed functionally complementing fragments.

FIG. 3

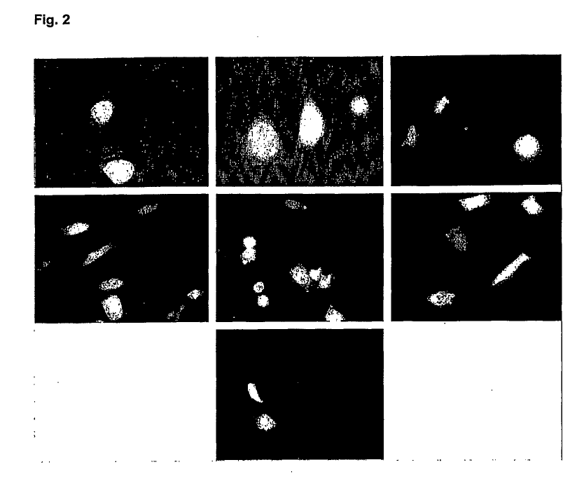

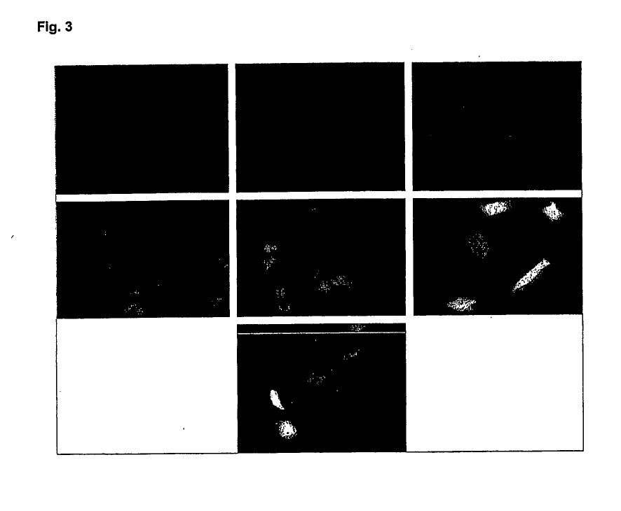

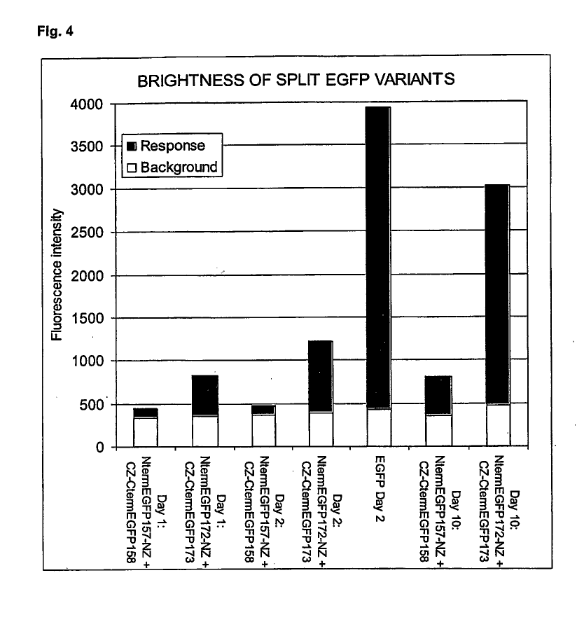

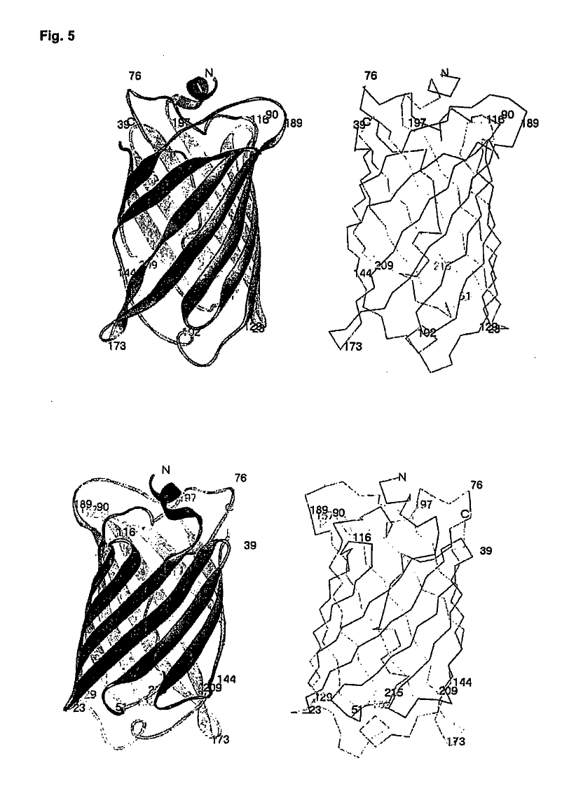

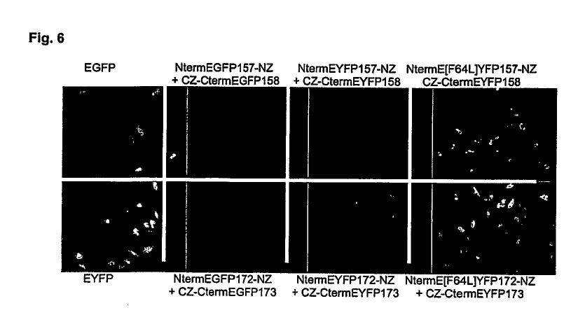

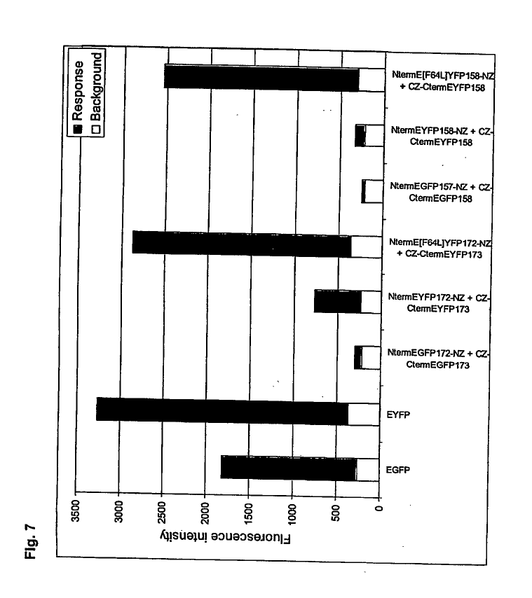

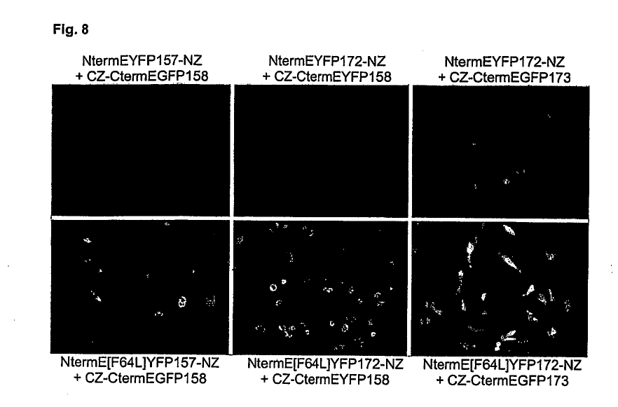

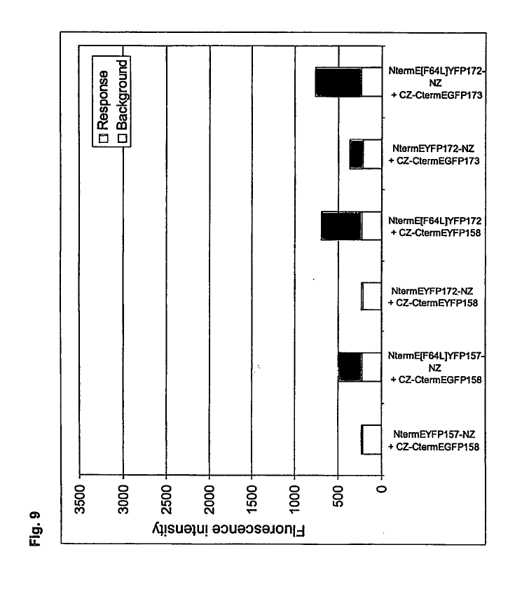

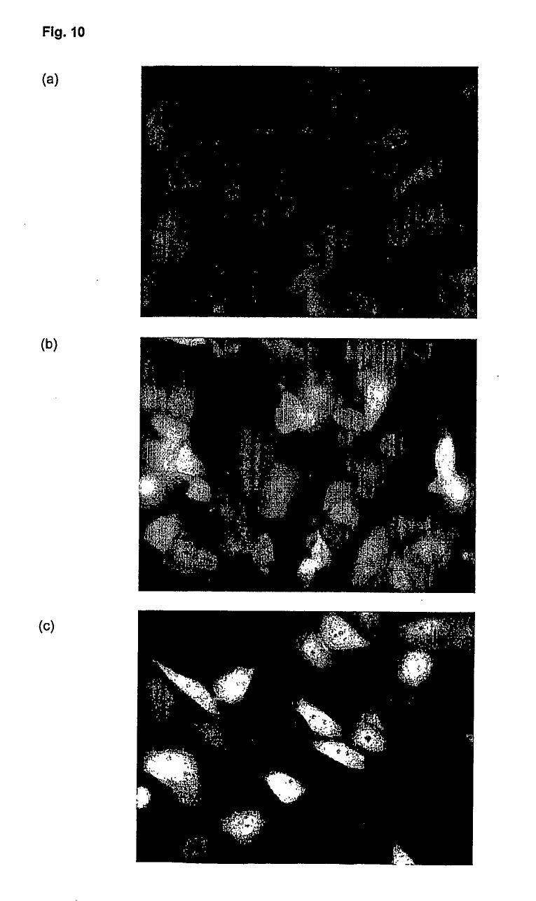

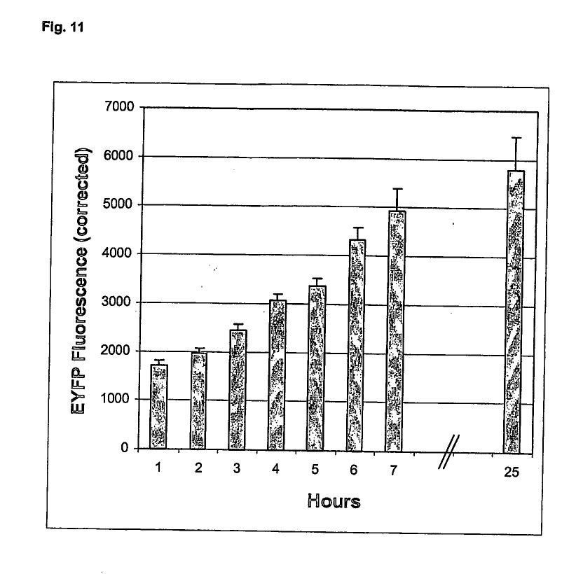

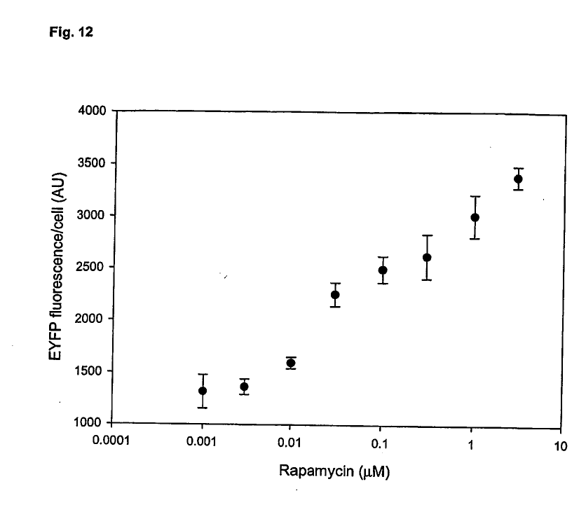

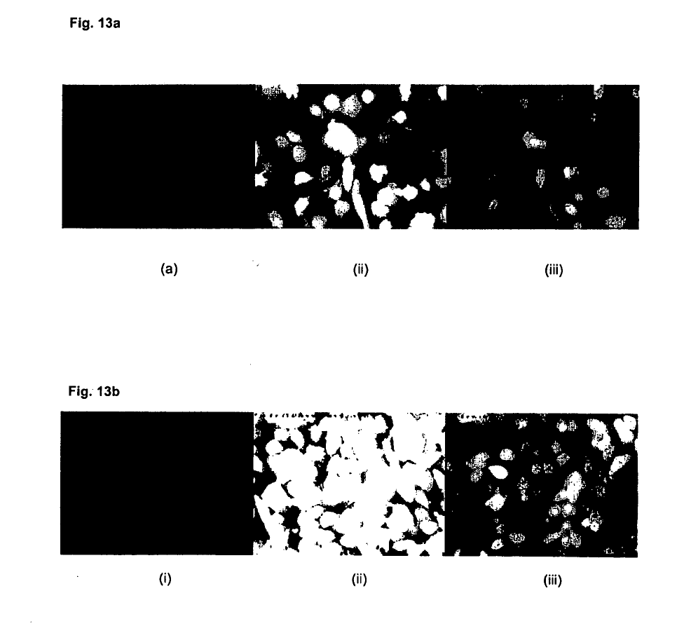

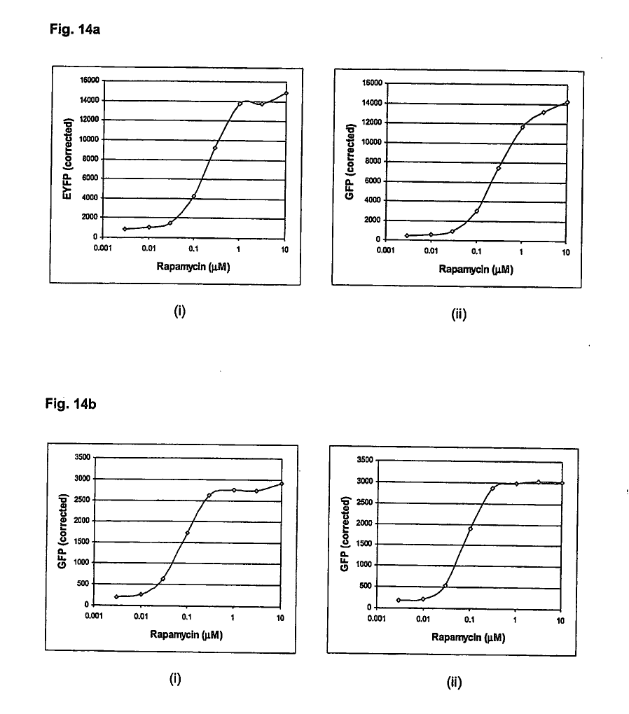

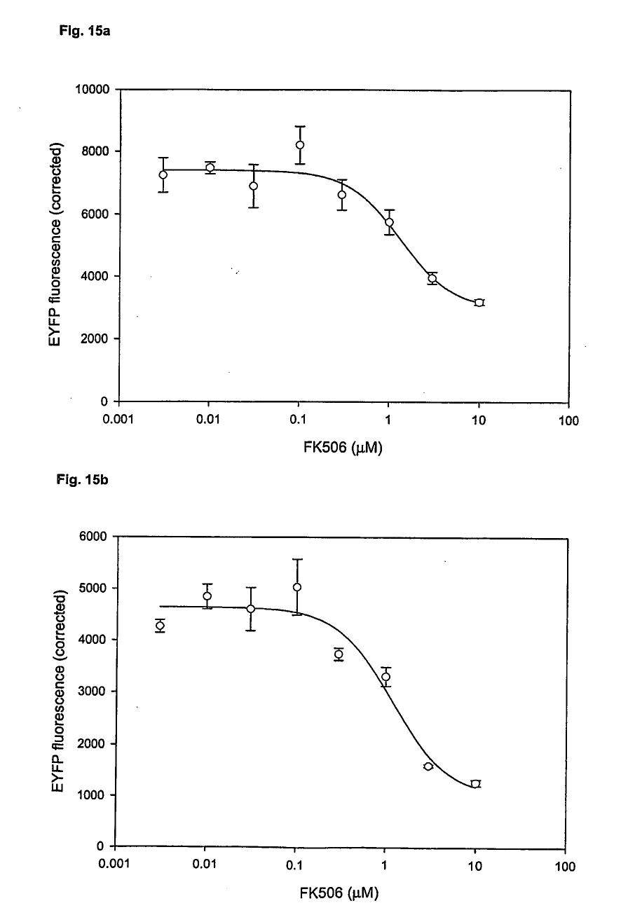

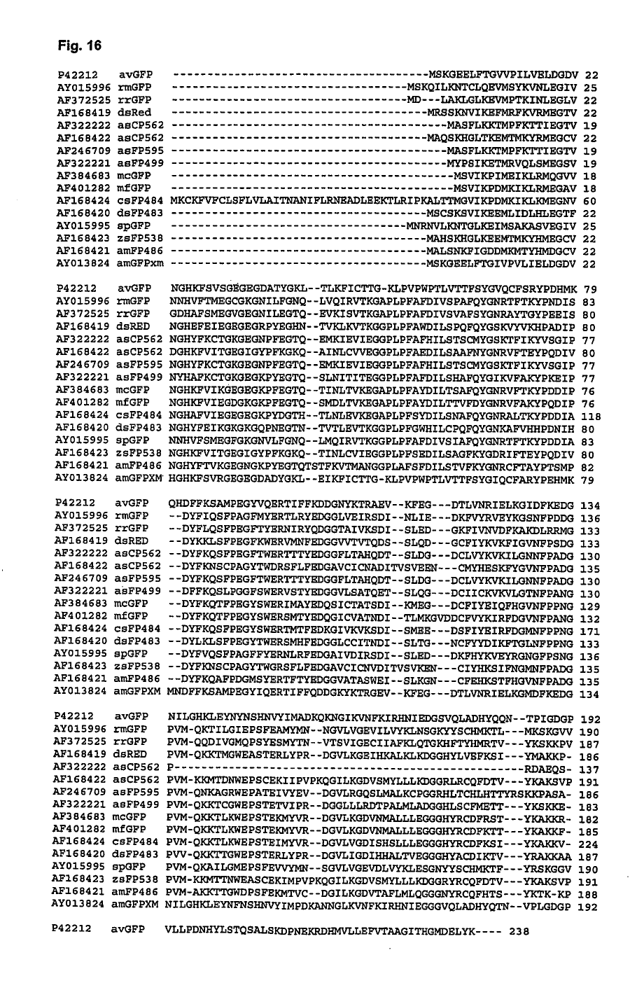

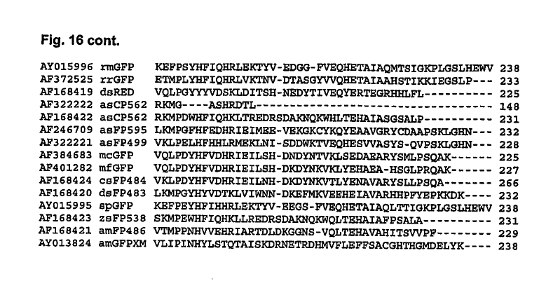

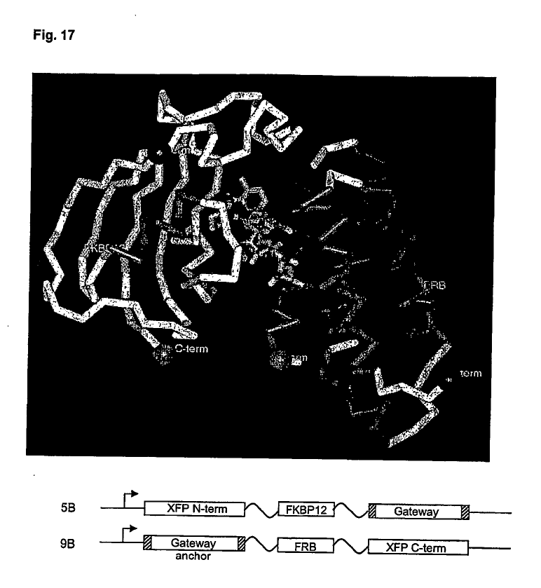

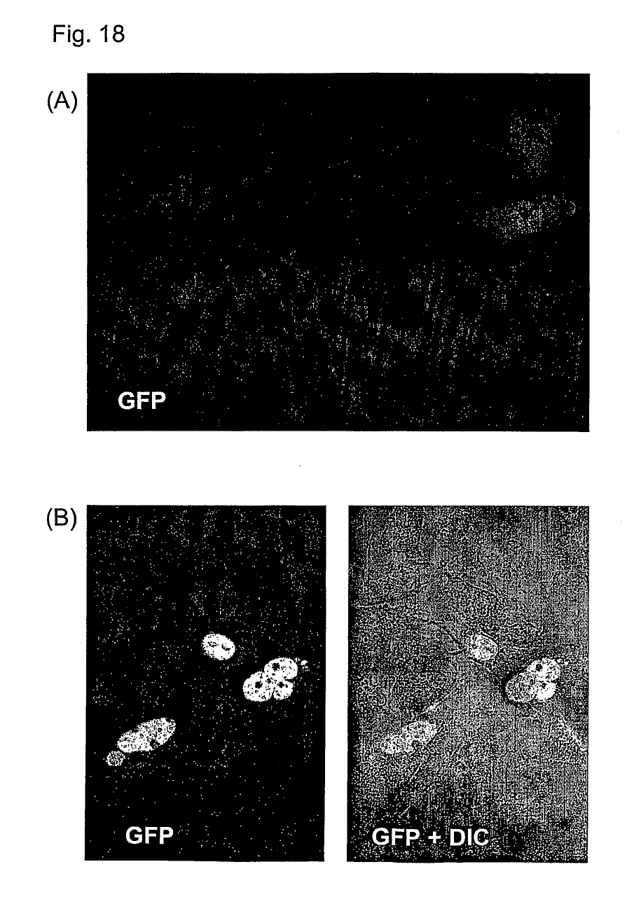

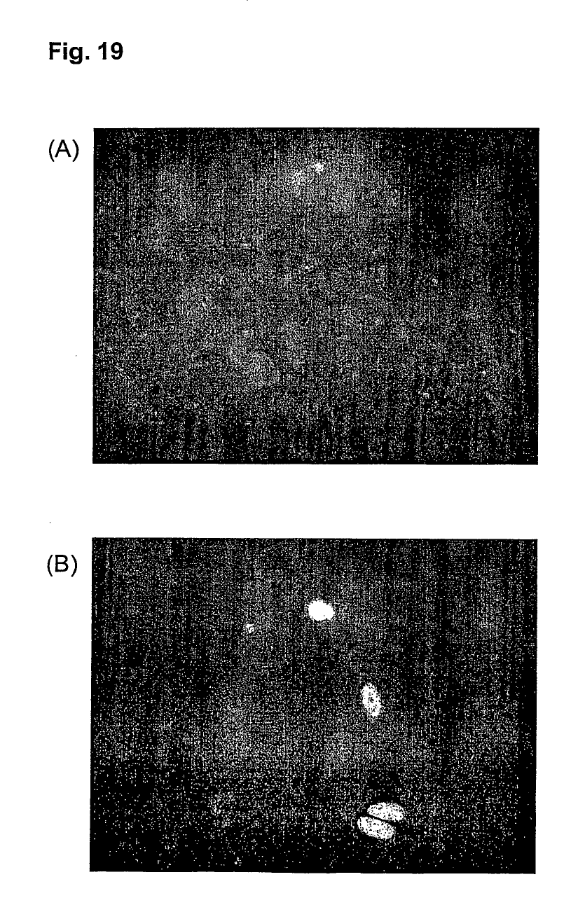

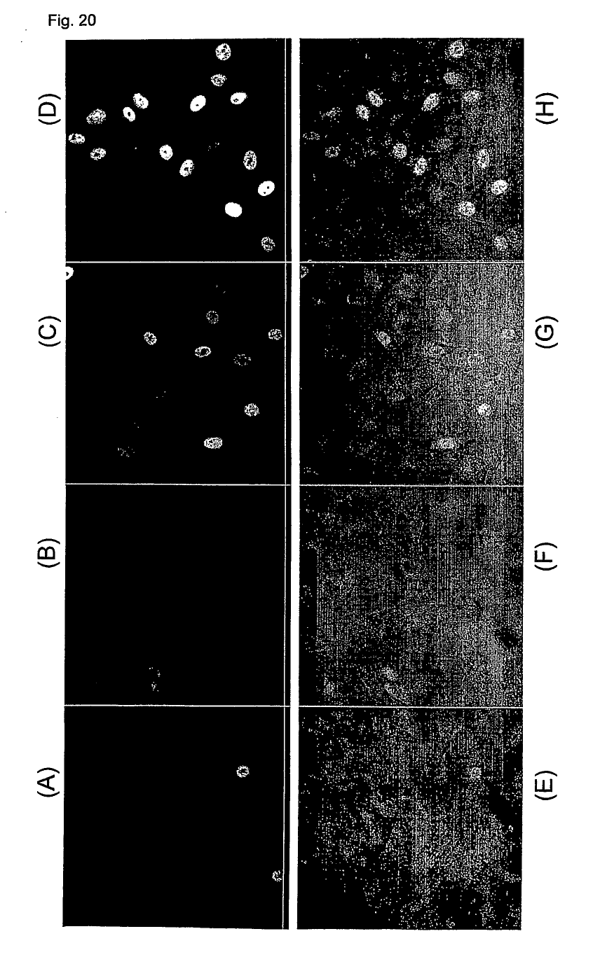

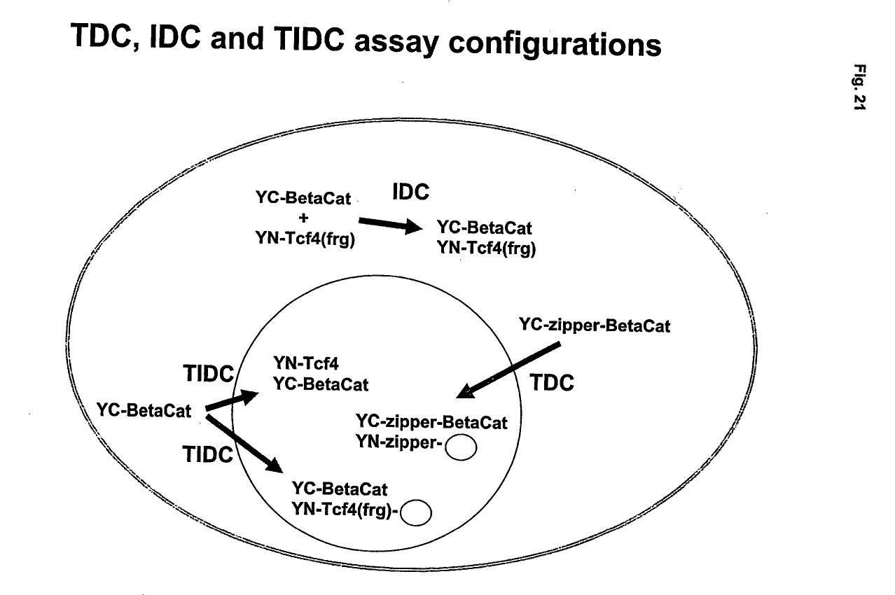

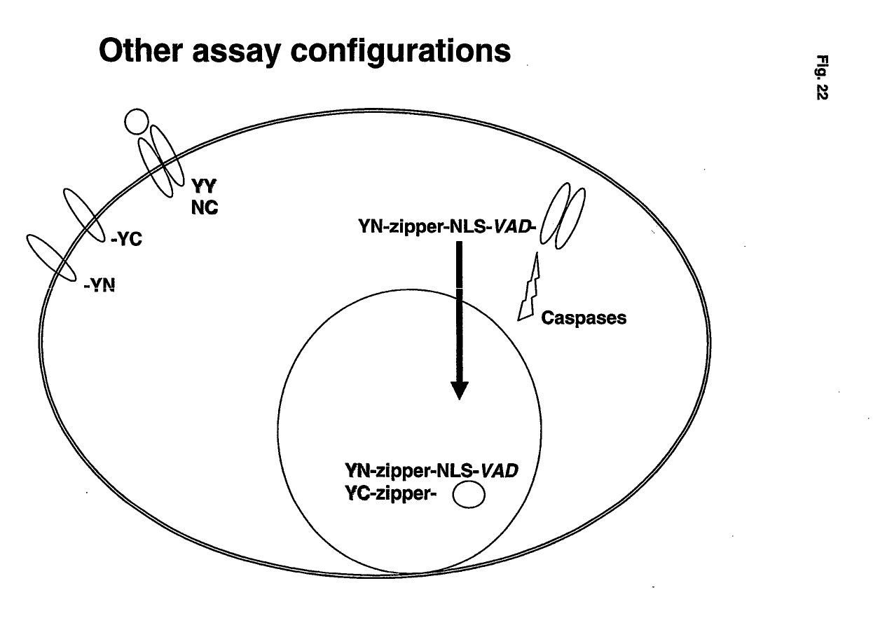

The same 16 bit images of fluorescent CHO-hIR cells co-transfected with NtermEGFP-NZ and CZ-CtermEGFP expression vectors or transfected with pEGFP-C1 as shown in FIG. 2 but the images are now shown with the same intensity scaling to allow comparison of fluorescence intensities. The cells that are transfected with complementation constructs that are based on a split between residues 172 and 173 (middle row) are clearly more fluorescent than the cells that are transfected with complementation constructs that are based on a split between residues 157 and 158 (top row). However, the cells transfected with the pEGFP-C1 construct (bottom row) show significantly stronger fluorescence at day 2.

FIG. 4

The unmanipulated microscope images shown in FIG. 3 were analysed using the ImageJ software package and data analysis was performed in Microsoft Excel. For each 16-bit monochrome IP Lab microscope image, pixel intensity data were produced in ImageJ and exported to an Excel spread-sheet for data analysis. The darkest and brightest 0.5% of the pixels were identified in each image and the average intensities of these two groups of pixels were calculated. The average intensity of the 0.5% darkest pixels was defined as the back ground fluorescence intensity (shown as white bars in the histogram) and the intensity of the 0.5% brightest pixels was defined as the maximum intensity. The difference in intensity between the maximum intensity and the background intensity was defined as the response (shown as cross hatched bars in the histogram). The sum of the background intensity and the response is equal to the maximum intensity. From the figure, it is clear that EGFP based fluorescence complementation using a split between residues 172 and 173, and probably anywhere else in this loop, is greatly superior to EGFP based fluorescence complementation using a split between residues 157 and 158 and probably also to splits anywhere else In this loop.

FIG. 5

Positions of appropriate fluorescent protein splitting sites are shown on ribbon and wire frame representations of GFP. The two representations show the same sites from two sides (molecule rotated approximately 180 degrees around a vertical axis).

FIG. 6

Co-transfection of expression vectors expressing EGFP and EYFP based complementation fragments as described in FIG. 3 to compare the abilities of the various complementation fragments to combine in cells and produce functional complexes. All images are scaled identically to allow direct comparison of fluorescence intensities between the images.

Single transfections with N-terminal fragments only resulted in no detectable fluorescence above the background level (data not shown). These N-terminal fragments contain amino acid residues 65-67 forming the chromophore in full-length GFP.

FIG. 7

Quantitative analysis of the images shown in FIG. 6. The results are in accord with the impressions from visual inspection of the cells. The data were produced as described in the legend to FIG. 4.

FIG. 8

Co-transfection of expression vectors expressing EGFP and EYFP based complementation fragments as described in FIG. 3 to compare the effects of mixing differently colored EGFP, EYFP and EYFP F64L fragments and to determine the influence of overlapping fragments, e.g. combining fragments encoding residues 1-172 and 158-238. All color combinations complement but typically less efficiently than in the correct combinations, i.e. when no or few residues overlap. Fragments having overlapping regions are also functional and this may be advantageous in experiments where longer linker sequences are or may be required by the fusion partners due to steric hindrance. This was not the case in this experiments where the fusion partners are leucine zippers. In the example (middle column), residues 158-172 were present in both fragments. In all situations, the F64L has a favorable effect on the fluorescence intensities. All images are scaled identically to allow direct comparison of fluorescence intensities between the images.

FIG. 9

Quantitative analysis of the images shown in FIG. 8. The results can be compared directly with the results shown in FIG. 7 and they are in accord with the impressions from visual inspection of the cells. The data were produced as described in the legend to FIG. 4.

FIG. 10

CHO-hIR [PS1767+PS1768] cells at 3 time points after treatment with 1 μM rapamycin.

Note that image (c) was taken at 25 msec exposure, the previous 2 images at exposures of 100 msec each.
(a) is the starting condition for these cells (t=0), and fluorescence is barely visible in most cells, although it was noted that some cells (<5%) in the population had low, but appreciable, fluorescence before treatment.
(b), after 4 hours many cells (approximately 40%) had developed significantly greater EYFP fluorescence throughout the cytoplasmic and nuclear locations.
   (c) after 16 hours the response per cell had Increased further and encompassed a larger proportion of the cell population (approximately 70%).

FIG. 11

The rate of development of cellular EYFP fluorescence following rapamycin treatment of the CHO-hIR [PS1767+PS1768] line. Cells were treated in 96-well plates with 3 μM rapamycin and the fluorescence measured. Treatment and measurements were made with the cells growing in HAM's medium+10% FBS, and fluorescence measurements were corrected for the background fluorescence from this medium. The graph demonstrates that the half-time for development of fluorescence is approximately 5 hours. Values corrected for HAM's background, each value a mean+sd for 8 measurements.

FIG. 12

Response curve to different rapamycin doses for the CHO-hIR [PS1769+PS1771] cell line. Cells were cultured in 96-well plates, treated with various rapamycin doses for 16 hours, then fixed and stained with Hoechst prior to determination of EYFP fluorescence/cell (arbitrary units) on the Ascent plate reader. Values are corrected for PBS background as well as cell number. The cell line shows approximately a 3-fold increase in the EYFP intensity/cell over the dose range of rapamycin used in this experiment.

FIG. 13

Each of the cell lines were fluorescence activated cell sorted (FACS) into 3 groups: (i) most green group (ii) medium to low-green group and (iii) black group. The ‘most green’ was discarded in each case, while the other 2 groups were cultured for further use.

A: CHO-hIR [ps1768+ps1767] FACS group ‘Black’ before stimulation (i), and after 16 hours stimulation with 100 nM rapamycin (ii) & (iii). Images (i) and (ii) were exposed for 100 msec, image (iii) for 25 msec.

B: CHO-hIR [ps1768+ps1767] FACS group ‘medium-low green’ before stimulation (i), and after 16 hours stimulation with 100 nM rapamycin (ii) & (iii). Images (i) and (ii) were exposed for 100 msec, image (iii) for 25 msec.

FIG. 14

Show the response of the ‘medium to low-green’ (a) and ‘black’ (b) FACS groups (respectively) derived from the CHO-hIR [PS1767+PS1768] parent line (see FIG. 13). Dose response to rapamycin was measured after 7 hours (i) and 30 hours (ii) for each cell line. Values for fluorescence have been corrected for plate & medium background. Increase in EYFP fluorescence is better than 20-fold the unstimulated value In each case.

FIG. 15

Show dose-response competition curves for FK506 versus 100 nM rapamycin in two of the FACS'd lines, (a) CHO-hIR [PS1768+PS1767] ‘mid to low-green’ group, and (b) CHO-hIR [PS1769+PS1771] ‘black’ group. EC₅₀ values in both cases are approximately 1.2 μM FK506. The cells were incubated overnight (16 hours) with mixtures of the two compounds, then fixed and stained with Hoechst prior to determination of EYFP fluorescence/cell on an Ascent plate reader. Plate and solution backgrounds have been subtracted; the dashed lines on each graph indicate the prestimulated fluorescence levels for each cell line in these experiments.

FIG. 16

Alignment of fluorescent proteins.

FIG. 17

Crystal structure of rapamycin induced binding of FRBP12 (left) bound to FRB (right). The C-termini of the molecules are within 15 Å of each other. The N-terminus of one of the molecules is 32-36 Å away from the C-terminus of the other molecule.

FIG. 18

(A) shows the level of fluorescence in stable CHO[ps1706+ps1810] (NtermE[F64L]YFP172-β-catenin+H2B-TCF4(frag)-CtermEYFP173) prior to any treatment of the cells.

The level of fluorescence is very weak in most cells and may be due entirely to cellular autofluorescence from flavanoids such as FAD (the emision filter used typically allows a fraction of FAD autofluorescence to pass). Occasional cells (top right corner of (A)) show a weak GFP fluorescence in nuclear and cytoplasmic locations, apparently the result of spontaneous complementation. Most of these cells are found clustered, possibly indicating a clonal behaviour that may reflect incorrect processing of the introduced components, such that they are brought together into the same location during expression (or possibly cell division) where complementation can occur. However, when treated for 16 hours with 5 mM LiCl, the number of cells containing fluorescent GFP increases greatly, and many of these have the GFP fluorescence confined exclusively to their nuclei consistent with the distribution of GFP complementation expected from this pair of fusion constructs (B).

FIG. 19

(A) shows the level of fluorescence in stable CHO[ps1736+ps1758] (NtermE[F64L]YFP172-Zip-H2B+β-catenin-Zip-CtermEYFP173 prior to any treatment of the cells. When treated for 16 hours with 5 mM LiCl, a fraction of these cells display GFP-bright nuclei, consistent with the distribution of GFP complementation expected from this pair of fusion constructs ((B))

FIG. 20

This figure shows a montage of CHO cells expressing ps1809 with ps1827 (NtermE[F64L]YFP172-FKBP-beta-Catenin+H2B-FRAP(2024-2114)-CtermEYFP173), imaged from 4 separate treatments. The top row (a-d) are images of GFP fluorescence. The lower row of images (e-h) where transmitted-light images have been overlayed with their corresponding GFP fluorescence.

a&e: No treatment.

b&f: 3 μM rapamycin (+6 hours)

c&g:16 hours with 5 mM lithium chloride

d&h:16 hours with 5 mM lithium chloride 3 μM rapamycin (+6 hours)

FIG. 21

Figure showing the various assay configurations.

IDC (Interaction Dependent Complementation) is the regular configuration where interaction between two proteins (here illustrated as BetaCat and Tcf4) will cause the N- and C-terminal XFP complementation partners to reassembly and be detectable. One particular advantage of IDC pairs is to validate TIDC assays developed from chosen protein pairs. The rapamycin inducible FKBP-FRB interaction pair can be used to validate and further develop the utility of IDC. See also the description of the TDCrap system.

TIDC (Translocation and Interaction Dependent Complementation) is the configuration where redistribution and interaction is measured as an increase in light intensity. Beta-catenin fused to a complementation fragment is predominantly located in the cytoplasm. Upon stimulation, beta-catenin will translocate to the nucleus. Here, it will find the natural interaction partner TCF-4 fused to the complementing fragment. The two complementation partners will assemble and become fluorescent. Compounds that inhibit either beta-catenin translocation or beta-catenin-TCF-4 interaction will inhibit complementation and formation of the fluorescent complex.

TDC (Translocation Dependent Complementation) is the configuration where only redistribution is measured as an increase in light intensity. For example, beta-catenin fused to a XFP complementation fragment and a leucine zipper sequence is predominantly located in the cytoplasm. Upon stimulation, beta-catenin fusion will translocate to the nucleus. Here, the leucine zipper will find the other leucine zipper sequence which is fused to the complementing XFP fragment and an anchor protein or nuclear localisation signal. The two complementation partners will assemble because of the zipper interactions and become fluorescent. Compounds that inhibit either beta-catenin translocation will inhibt complementation and formation of the fluorescent complex.

TDCrap (Translocation Dependent Complementation induced by rapamycin) is basically the same as TDC but the use of an inducible interaction pair, e.g. the rapamycin inducible FKBP and FRB interaction pair, greatly reduces the “sink effect” and facilitates the development of assays based on other types of translocation, e.g. from the cytoplasm to plasma membrane or visa versa.

FIG. 22

The left hand part of this figure shows the assay configuration for determining dimerization (in this case homo-dimerization) of receptors. Two conjugates comprising the receptor and the N-terminus al of the complementation protein (here YN) or the C-terminus al of the complementation protein (here YC) are expressed in the cell. Only upon stimulation of the cell with a ligand, the receptors are brought in close apposition and the YN and YC terminal fragments will from a functional protein.

The right hand side of the figure illustrates the configuration of an assay for caspase activity. The first conjugate comprises the N-terminus al of the complementation protein (YN), a constitutive interaction partner (a leucine zipper), a nuclear localisation sequence (NLS), a consensus cleavage site for caspases (e.g. VAD=Valine-Alanine-Aspartate) fused to an anchor protein, here in the cell membrane. The second conjugate comprises the N C-terminus al of the complementation protein (YC), the interaction partner (a leucine zipper) and an anchor in the nucleus (here marked as a circle). When caspase activity is increased, the caspases will cleave the YN fusion at the VAD site and the first conjugate will, due to the NLS, translocate to the nucles, where binding to the interaction partners (zippers), will cause fluorescence.

Tables

TABLE 2

Oligo nucleotides used in cloning. Oligo nucleotides beginning with
P* are phosphorylated at the 5′ end to permit ligation.

| | | |
|---|---|---|
| Oligo | | SEQ |
| nucleo- | | ID |
| tide | Oligo nucleotide sequence (5′ end to 3′ end) | NO: |

| | | |
|---|---|---|
| 1268 | ATTB2- CCTACTGCTTTGAGATTCGTCGG | 15 |
| 1272 | ATTB2- GTCATTCCAGTTTTAGAAGCTC | 16 |
| 1282 | CAGACAATCTGTGTGGGCACTCGACCGG | 17 |
| 2110 | P*CATGGCCGGTGGTACCGGTTCCGGTGCCCTGAAGAAGGAGCTGCAGG | 18 |
| 2111 | P*AGCTCCTTCTTCAGGGCACCGGAACCGGTACCACCGGC | 19 |
| 2112 | P*CCAACAAGAAGGAGCTGGCCCAGCTGAAGTGGGAGCTGCAG | 20 |
| 2113 | P*CTCCCACTTCAGCTGGGCCAGCTCCTTCTTGTTGGCCTGC | 21 |
| 2114 | P*GCCCTGAAGAAGGAGCTGGCCCAGTAG | 22 |
| 2115 | P*GATCCTACTGGGCCAGCTCCTTCTTCAGGGCCTGCAG | 23 |
| 2116 | P*CATGGCCAGCGAGCAGCTGGAGAAGAAGCTGCAGGCCCTG | 24 |
| 2117 | P*CCTGCAGCTTCTTCTCCAGCTGCTCGCTGGC | 25 |
| 2118 | P*GAGAAGAAGCTGGCCCAGCTGGAGTGGAAGAACCAGGCCCTGGAG | 26 |
| 2119 | P*GGCCTGGTTCTTCCACTCCAGCTGGGCCAGCTTCTTCTCCAGGG | 27 |
| 2120 | P*AAGAAGCTGGCCCAGGGCGGCACCGGTTAG | 28 |
| 2121 | P*GATCCTAACCGGTGCCGCCCTGGGCCAGCTTCTTCTCCAG | 29 |
| 2128 | GGCGCCATGGTGAGCAAGGGCGAG | 30 |
| 2129 | GCCGGACCGGTACCACCGTTGTACTCCAGCTTGTG | 31 |
| 2130 | GCCGGACCGGTACCACCCTGCTTGTCGGCCATG | 32 |
| 2131 | GCCGGACCGGTACCACCCTCGATGTTGTGGCGGATC | 33 |
| 2132 | CCCCGGATCCTACTTGTACAGCTCGTCCATGC | 34 |
| 2133 | GGCGCCATGGGCACCGGTTACAACAGCCACAACGTC | 35 |
| 2134 | GGCGCCATGGGCACCGGTAAGAACGGCATCAAGGTG | 36 |
| 2135 | GGCGCCATGGGCACCGGTGACGGCAGCGTGCAGCTC | 37 |
| 2219 | GGGGGCTAGCGCCACCATGGTGAGCAAGGGCGAG | 38 |
| 2222 | GCGGGGGATCCGATATCGCCAGAGCCAGAGCCAGAGCCCTCGATGTTGTGGCGGATC | 39 |
| 2225 | GGGGGCTAGCGATATCCGGCTCTGGCTCTGGCTCTGGCGACGGCAGCGTGCAGCTC | 40 |
| 2333 | GCCCACCCTCGTGACCACCTTCGGCTACGGCCTACGTGCTTCGCCCGCTACCCCGACCACATG | 41 |
| 2334 | CATGTGGTCGGGGTAGCGGGCGAAGCACTGCAGGCCGTAGCCGAAGGTGGTCACGAGGGTGGGC | 42 |
| 2335 | GCCCACCCTCGTGACCACCCTGGGCTACGGCCTGCAGTGCTTCGCCCGCTACCCCGACCACATG | 43 |
| 2336 | CATGTGGTCGGGGTAGCGGGCGAAGCACTGCAGGCCGTAGCCCAGGGTGGTCACGAGGGTGGGC | 44 |
| 2337 | GACAACCACTACCTGAGCTACCAGTCCGCCCTGAGC | 45 |
| 2338 | GCTCAGGGCGGACTGGTAGCTCAGGTAGTGGTTGTC | 46 |
| 2442 | ATTB1- CCACCATGGGAGTGCAGGTGGAAACC | 47 |
| 2443 | ATTB2- CTTCCAGTTTTAGAAGCTC | 48 |
| 2444 | ATTB1- CCACCATGGAGATGTGGCATGAAGGCCTG | 49 |
| 2445 | ATTB2- CCTGCTTTGAGATTCGTCGGAACAC | 50 |
| 9655 | TCCTAGGTCAGTCCTGCTCCTCGGCCACGAAGTGCACTCCTAGGCTGCAGCACGTGTTGACAATTAATCATCGG | 51 |
| 9658 | CAGACAATCTGTGTGGGCACTCGACCGG | 52 |
| 2387 | GCGGGGGATCCGATATCGCCAGAGCCAGAGCCAGAGCCctgggccagctccttcttcag | 53 |
| 2368 | attB2- cctacttggagctggtgtacttggtgac | 54 |
| 2388 | GGGGGCTAGCGATATCCGGCTCTGGCTCTGGCTCTGGCGACGGCAGCGTGCAGCTC | 55 |
| 2359 | attB1- ccaccatggctactcaagctgatttgatg | 56 |
| 2439 | attB2- ccaggtcagtatcaaaccaggccag | 57 |
| 2360 | attB2- cctacaggtcagtatcaaaccaggccag | 58 |
| 2389 | gttgctagcaccatgcctgagccagccaagtctgct | 59 |
| 2390 | gttgatatcccttggagctggtgtacttggtgac | 60 |
| 2364 | attB1q- ccaccatgccgcagctgaacggcggtgga | 61 |
| 2366 | attB2- caggcggaggccgtctttccgc | 62 |
| 2462 | gttcagctgatgggagtgcaggtggaaaccatctcc | 63 |
| 2463 | gttggatccgatatcaccggtgcctgtcccagttccttccagttttagaagctccacatc | 64 |
| 2464 | gttgctagcgatatccggaacgggcactgggaccggtgagatgtggcatgaaggcctggaag | 65 |
| 2465 | gttcccgggagctgctttgagattcgtcggaacac | 66 |
| 2367 | attB1- ccaccatgcctgagccagccaagtctgct | 67 |
| 2407 | attB2- ccttggagctggtgtacttggtgac | 68 |

TABLE 3
Primer pairs used in EGFP fragment amplification

| Protein encoded by PCR fragment | 5′ primer | 3′ primer |
|---|---|---|
| EGFP(1-144) | 2128 | 2129 |
| EGFP(1-157) | 2128 | 2130 |
| EGFP(1-172) | 2128 | 2131 |
| EGFP(145-238) | 2133 | 2132 |
| EGFP(158-238) | 2134 | 2132 |
| EGFP(173-238) | 2135 | 2132 |

TABLE 4
Cloning and expression vectors

| | | | Selection |
|---|---|---|---|
| Vector | Expressed protein | Promotor | E. coli/mamm. |
| pEGFP-C1 | EGFP | CMV | kan/neo |
| PS0609 | EGFP | CMV | zeo/zeo |
| pTrcHis-A | no insert | Trc | amp/none |
| PS1515 | NZ leucine zipper | Trc | amp/none |
| PS1516 | CZ leucine zipper | Trc | amp/none |
| PS1614 | NtermEGFP144-NZ | Trc | amp/none |
| PS1596 | NtermEGFP157-NZ | Trc | amp/none |
| PS1597 | NtermEGFP172-NZ | Trc | amp/none |
| PS1615 | CZ-CtermEGFP145 | Trc | amp/none |
| PS1594 | CZ-CtermEGFP158 | Trc | amp/none |
| PS1595 | CZ-CtermEGFP173 | Trc | amp/none |
| PS1559 | NtermEGFP157-NZ | CMV | kan/neo |
| PS1560 | NtermEGFP172-NZ | CMV | kan/neo |
| PS1557 | CZ-CtermEGFP158 | CMV | zeo/zeo |
| PS1558 | CZ-CtermEGFP173 | CMV | zeo/zeo |
| PS1639 | NtermEYFP157-NZ | CMV | kan/neo |
| PS1642 | NtermEYFP172-NZ | CMV | kan/neo |
| PS1640 | (NtermE[F64L]157YFP-NZ | CMV | kan/neo |
| PS1641 | NtermE[F64L]YFP172-NZ | CMV | kan/neo |
| PS1637 | CZ-CtermEYFP158 | CMV | zeo/zeo |
| PS1638 | CZ-CtermEYFP173 | CMV | zeo/zeo |
| PS1769 | NtermE[F64L]YFP172-FKBP | CMV | kan/neo |
| PS1767 | NtermE[F64L]YFP172-FRB | CMV | kan/neo |
| PS1771 | FRB-CtermEYPF173 | CMV | zeo/zeo |
| PS1768 | FKBP-CtermEYFP173 | CMV | zeo/zeo |

TABLE 5
Sequence names and numbers

| SEQ ID NO: | Name |
|---|---|
| 1 | Amino acid sequence of GFP |
| 2 | Amino acid sequence of GFP Y66W |
| 3 | Amino acid sequence of GFP Y66H |
| 4 | Amino acid sequence of EGFP |
| 5 | Amino acid sequence of EYFP |
| 6 | Amino acid sequence of EYFP F64L variant |
| 7 | Nucleic acid sequence of NZ |
| 8 | Amino acid sequence of NZ |
| 9 | Nucleic acid sequence of CZ |
| 10 | Amino acid sequence of CZ |
| 11 | NtermE[F64L]YFP172 and FKBP linker sequence |
| 12 | NtermE[F64L]YFP172 and FRB linker sequence |
| 13 | FRB and CtermEYFP173 linker sequence |
| 14 | FKBP and CtermEYFP173 linker sequence |
| 15-68 | Primer sequence (see Table 2) |
| 69 | beta-Catenin and CZ-CtermEYFP173 linker sequence |
| 70 | NtermE[F64L]YFP172 and beta-Catenin linker sequence |
| 71 | TCF4(1-70) and CtermEYFP173 linker sequence |
| 72 | NtermE[P64L]YFP172 and FKBP and beta-Catenin |
| 73 | H2B and FRAP (amino acids 2025-2114) linker sequence |
| 74 | NtermE[F64L]YFP172-NZ and H2B linker sequence |
| 75 | H2B and TCF4(1-70) linker sequence |
| 76 | FKBP and beta-Catenin linker sequence |

All cited patens, publications, copending applications, and provisional applications referred to in this application are herein incorporated by reference.

The invention being thus described, it will be obvious that the same may be varied in many ways. Such avariations are not to be regarded as a departure from the spirit and scope of the present inventions, and all such modifications as would be obvious to one skilled in the art are intended to be included within the scope of the following claims.

1. A cell comprising at least:
a first conjugate comprising a first protein and the N-terminal fragment of complementation protein; and
a second conjugate comprising a second protein and the C-terminal fragment of the complementation protein;
wherein said first conjugate has a predominant location in a different cellular location from where said second conjugate is predominantly located,
wherein said first protein and said second protein will bind to each other, and
wherein said complementation protein exhibits altered characteristics when the two fragments of the complementation protein are brought into close apposition and the two fragments of the complementation protein form the full functional protein.

2. The cell according to any claim 1, wherein the cellular compartment of the first conjugate is determined by the nature of the first protein.

3. The cell according to claim 1, wherein the cellular compartment of the second conjugate is determined by the nature of the second protein.

4. The cell according to claim 1, wherein the second conjugate further comprises an anchor protein, wherein said anchor protein is anchored in a different cellular compartment from where said first protein is predominantly located.

5. A cell comprising at least:
a first conjugate comprising a first protein, interaction partner A and the first terminal fragment of a complementation protein; and
a second conjugate comprising a interaction partner B and the second terminal fragment of the complementation protein;
wherein said first conjugate has a predominant location in a different cellular compartment from where said second conjugate is predominantly located,
wherein said complementation protein exhibits altered characteristics when the two fragments of the complementation protein are brought into close apposition and the two fragments of the complementation protein form the full functional protein.

6. The cell according to claim 5, wherein said interaction partner A and interaction partner B bind to each other.

7. The cell according to claim 5, wherein said interaction partner A and interaction partner B bind to each other only when an interaction stimulus has been applied.

8. The cell according to claim 5, wherein the second conjugate further comprises an anchor protein, wherein said anchor protein is anchored in a different cellular compartment from where said first protein is predominantly located.

9. The cell according to claim 5, wherein complementation protein is dihydrofolate reductase (DHFR).

10. The cell according to claim 1, wherein the complementation protein is Ubiquitin.

11. The cell according to claim 1, wherein complementation protein is β-lactamase.

12. The cell according to claim 1, wherein complementation protein is β-galactosidase.

13. The cell according to claim 1, wherein complementation protein is a fluorescent protein.

14. The cell according to claim 13, wherein the complementation protein is Green Fluorescent Protein (GFP).

15. The cell according to claim 14, wherein the complementation protein is derived from Aequoria Victoria.

16. The cell according to claim 14, wherein N-terminal fragment of the complementation protein is an N-terminal fragment of GFP, comprising a continuous stretch of amino acids from amino acid number 1 to amino acid number X of GFP, wherein the peptide bond between amino acid number X and amino acid number X+1 is within a loop of GFP and C-terminal fragment of the complementation protein is a C-terminal fragment of GFP, comprising a continuous stretch of amino acids from amino acid number X+1 to amino acid number 238 of GFP.

17. The cell according to claim 14, wherein the N-terminal fragment of the complementation protein is an N-terminal fragment of GFP, comprising a continuous stretch of amino acids from amino acid number 1 to amino acid number X of GFP, wherein the peptide bond between amino acid number X and amino acid number X+1 is within a loop of GFP and the C-terminal of the complementation protein is a C-terminal fragment of GFP, comprising a continuous stretch of amino acids from amino acid number Y+1 to amino acid number 238 of GFP, wherein Y<X creating an overlap of the two GFP fragments, and wherein the peptide bond between amino acid Y and amino acid Y+1 is within a loop of GFP.

18. The cell according to claim 14, wherein GFP is selected from the group consisting of EGFP, EYFP, ECFP, dsRed and Renilla GFP.

19. The cell according to claim 18, wherein the GFP is EGFP.

20. The cell according to claim 18, wherein the GFP is EYFP.

21. The cell according to claim 14, wherein the amino acid in position 1 preceding the chromophore has been mutated to provide an increase of fluorescence intensity.

22. The cell according to claim 21, wherein the amino acid F in position 1 preceding the chromophore has been substituted by L.

23. The cell according to claim 14, wherein the GFP has been mutated to further contain the S72A mutation.

24. The cell according to claim 16, wherein X is between 9 and 10 within the Thr9-Val11 loop; or between 23 and 24 within the Asn23-His25 loop; or between 38 and 39 within the Thr38-Gly40 loop; or between 48 and 55 within the Cys48-Pro56 loop; or between 72 and 75 within the Ser72-Asp76 loop; or between 81 and 82 within the His81-Phe83 loop; or between 88 and 89 within the Met88-Glu90 loop; between 101 and 102 within the Lys101-Asp103 loop; or between 114 and 117 within the Phe114-Thr118 loop; or between 128 and 144 within the Ile 128-Tyr145 loop; or between 154 and 159 within the Ala154-Gly160 loop; or between 171 and 174 within the Ile171-Ser175 loop; or between 188 and 196 within the Ile188-Asp197 loop; or between 210 and 214 within the Asp210-Art215 loop.

25. The cell according to claim 24, wherein X is between 154 and 159 within the Ala154-Gly160 loop.

26. The cell according to claim 25, wherein X is 157 within the Ala154-Gly160 loop.

27. The cell according to claim 24, wherein X is between 171 and 174 within the Ile171-Ser175 loop.

28. The cell according to claim 27, wherein X is 172 within in Ile171-Ser175 loop.

29. The cell according to claim 17, wherein Y is between 154 and 159 within the Ala154-Gly160 loop.

30. The cell according to claim 29, wherein Y is 157 within the Ala154-Gly160 loop.

31. The cell according to claim 16, wherein X is 172 within in Ile171-Ser175 loop and wherein Y is 157 within the Ala154-Gly160 loop.

32. The cell according to claim 1, wherein the N-terminal fragment of the complementation protein is fused in frame with a first protein of interest.

33. The cell according to claim 1, wherein the first protein is fused to the N-terminal of the N-terminal fragment of the complementation protein.

34. The cell according to claim 1, wherein the first protein is fused to the C-terminal of the N-terminal fragment of the complementation protein.

35. The cell according to claim 1, wherein the C-terminal fragment of the complementation protein is fused in frame with the second protein.

36. The cell according to claim 1, wherein the second protein of interest is fused to the N-terminal of the C-terminal fragment of the complementation protein.

37. The cell according to claim 1, wherein the second protein of interest is fused to the C-terminal of the C-terminal fragment of the complementation protein.

38. The cell according to claim 1, wherein the N-terminal fragment of the complementation protein fused in frame to a first protein further comprises a linker sequence between the N-terminal fragment of the complementation protein and the first protein.

39. The cell according to claim 1, wherein the C-terminal fragment of the complementation protein fused in frame to a second protein further comprises a linker sequence between the C-terminal fragment of the complementation protein and the second protein.

40. The cell according to claim 14, wherein the GFP is EYFP further containing an F64L mutation, wherein X is 172, wherein the first protein fused to the N-terminal fragment of GFP is fused to the C-terminal of the N-terminal fragment of GFP and wherein the second protein fused to the C-terminal fragment of GFP is fused to the N-terminal of the C-terminal fragment of GFP.

41. The cell according to claim 14, wherein the GFP is EYFP further containing an F64L mutation, wherein X is 157, wherein the first protein fused to the N-terminal fragment of GFP is fused to the C-terminal of the N-terminal fragment of GFP and wherein the second protein fused to the C-terminal fragment of GFP is fused to the N-terminal of the C-terminal fragment of GFP.

42. The cell according to claim 1, wherein the first conjugate is expressed in the cell.

43. The cell according to claim 1, wherein the second conjugate is expressed in the cell.

44. The cell according to claim 5, wherein interaction partner A is FKBP12, interaction partner B is FRAP (or vise versa) and the interaction stimulus is Rapamycin.

45. The cell according to claim 5, wherein interaction partner A is FKBP12, interaction partner B is FRB (T2098L) (or vise versa) and the interaction stimulus is Rapamycin.

46. The cell according to claim 5, wherein interaction partner A is FKBP12, interaction partner B is FRB (T2098L) (or vise versa) and the interaction stimulus is AP21967.

47. The cell according to claim 5, wherein interaction partner A is FKBP12, interaction partner B is FKBP12 (or vise versa) and the interaction stimulus is AP21967.

48. The cell according to claim 5, wherein interaction partner A is FKBP12, interaction partner B is FKBP12 (or vise versa) and the interaction stimulus is AP21967.

49. The cell according to claim 5, wherein interaction partner A and B are steroid hormone receptors and the dimerizing agent is the cognate hormone ligand.

50. The cell according to claim 5, wherein interaction partner A and B are estrogen receptors and the interaction stimulus agent is estrogen.

51. The cell according to claim 4, wherein the anchor protein is a protein containing the transmembrane domain of the epidermal growth factor receptor (EGFR), or containing the transmembrane domain of one of the integrin protein family, or containing the myristoylation sequence from c-Src (residues 1-14).

52. The cell according to claim 4, wherein the anchor protein is a histone protein or a protein normally restricted to nucleoli, for example the p120 nucleolar protein.

53. The cell according to claim 4, wherein the anchor protein is a protein normally confined to mitochondrial outer or inner membranes for example VDAC, F₀ subunit of ATP-ase, or NADH dehydrogenase.

54. The cell according to claim 4, wherein the anchor protein is a protein normally confined to the various different regions of Golgi bodies for example TGN38 or ADAM12-L.

55. The cell according to claim 4, wherein the anchor protein is a protein normally confined to focal adhesion complexes for example P125, FAK, integerin alpha or beta, or paxillin.

56. The cell according to claim 4, wherein the anchor protein is a protein normally associated with cytoskeletal structures such as F-actin strands or micro tubular bundles for example MAP4, actin binding domain of alpha-actinin, kinesins, myosins or dyniens.

57. The cell according to claim 4, wherein the anchor protein is a protein normally associated with nuclear material or nuclear components, such as histone proteins, including histones H1, H2A, H2B, H3, H4, and variants thereof.

58. The cell according to claim 4, wherein the anchor protein is a protein normally associated with nuclear membrane such as A and B type lamins, or associated with nuclear bodies such as splicing bodies, Cajal bodies, PML nuclear bodies (PML oncogenic domains, PODS), or transcription engines such as RNA polymerase POL-II.

59. A cell according to claim 1, where in the cell is an eukaryotic cell.

60. A cell according to claim 1, where in the cell is a prokaryotic cell.

61. A method for detecting protein-protein interactions comprising the steps of:
(a) providing a cell comprising at least:
a first conjugate comprising the first protein of interest and the first terminal fragment of a complementation protein; and
a second conjugate comprising the second protein of interest and the second terminal fragment of a complementation protein;
wherein said second protein of interest has a predominant location in a different cellular location from where said first protein of interest is predominantly located,
(b) determine if complementation partner A and complementation partner B has complemented;
complementation between complementation partner A and complementation partner B being indicative that the first protein of interest has translocated from the predominant location of this first protein of interest to the cellular compartment where the second protein of interest is located and that the two proteins of interest interact.

62. A method for detecting protein-protein interactions comprising the steps of:
(a) providing a cell according to claim 1, and
(b) determine if complementation partner A and complementation partner B has complemented;
complementation between complementation partner A and complementation partner B being indicative that the first protein of interest has translocated from the predominant location of this first protein of interest to the cellular compartment where the second protein of interest is located and that the two proteins of interest interact.

63. A method for testing if a compound prevents a protein-protein interaction comprising the steps of:
(a) providing a cell according to claim 1;
(a1) adding the compound to the cells of step (a);
(a2) adding a translocation stimulus to the cells of step (a1),
wherein the translocation stimulus causes the two conjugates to have the same cellular localization;
(b) determine if complementation partner A and complementation partner B has complemented;
less complementation compared to a system omitting step (a1) being indicate that the compound is capable of preventing the interaction between the two proteins.

64. A method according to claim 61, wherein the second conjugate further comprises an anchor protein, wherein said anchor protein is anchored in a different cellular compartment from where said first protein of interest is predominantly located.

65. A method for detecting translocation of a protein comprising the steps of:
(a) providing a cell comprising at least:
a first conjugate comprising said protein, the first terminal of the complementation protein, and interaction partner A; and
a second conjugate comprising an anchor protein, the second terminal of complementation protein, and interaction partner B;
wherein said anchor protein is anchored in a different cellular compartment from where said protein is predominantly located;
and wherein said interaction partner A and interaction partner B bind to each other;
(b) determine if complementation partner A and complementation partner B has complemented;
complementation between complementation partner A and complementation partner B being indicative that said protein has translocated from the predominant location of the protein to the cellular compartment where the anchor protein is anchored.

66. A method for detecting translocation of a protein comprising the steps of:
(a) providing a cell according to claim 6, and
(b) determine if complementation partner A and complementation partner B has complemented;
complementation between complementation partner A and complementation partner B being indicative that said protein has translocated from the predominant location of the protein to the cellular compartment where the anchor protein is anchored.

67. A method for testing if a compound induces translocation of a protein comprising the steps of:
(a) providing a cell according to claim 6;
(a1) adding the compound to the cells of step (a);
(b) determine if complementation partner A and complementation partner B has complemented;
ncreased complementation compared to a system omitting step (a1) being indicate that the compound induces translocation of the protein.

68. A method for testing if a compound prevents translocation of a protein comprising the steps of:
(a) providing a cell according to claim 6;
(a1) adding the compound to the cells of step (a);
(a2) adding a translocation stimulus to the cells of step (a1):
(b) determine if complementation partner A and complementation partner B has complemented;
less complementation compared to a system omitting step (a1) being indicative that the compounds prevents translocation of the protein.

69. A method for detecting translocation of a protein comprising the steps of:
(a) providing a cell comprising at least:
a first conjugate comprising said protein, complementation partner A, and interaction partner A; and
a second conjugate comprising an anchor protein, complementation partner B, and interaction partner B;
wherein said anchor protein is anchored in a different cellular compartment from where said protein is predominantly located,
wherein said interaction partner A and interaction partner B bind to each other only when an interaction stimulus has been applied;
(b) add an interaction stimulus;
(c) determine if complementation partner A and complementation partner B has complemented;
complementation between complementation partner A and complementation partner B being indicative that said protein has translocated from the predominant location of the protein to the cellular compartment where the anchor protein is anchored after addition of the interaction stimulus.

70. A method for detecting translocation of a protein comprising the steps of:
(a) providing a cell according to claim 7, and
(b) add an interaction stimulus;
(c) determine if complementation partner A and complementation partner B has complemented;
complementation between complementation partner A and complementation partner B being indicative that said protein has translocated from the predominant location of the protein to the cellular compartment where the anchor protein is anchored after addition of the interaction stimulus.

71. A method for testing if a compound induces translocation of a protein comprising the steps of:
(a) providing a cell according to claim 7;
(a1) adding the compound to the cells of step (a);
(b) add an interaction stimulus;
(c) determine if complementation partner A and complementation partner B has complemented;
increased complementation compared to a system omitting step (a1) being indicate that the compound induces translocation of the protein.

72. A method for testing if a compound prevents translocation of a protein comprising the steps of:
(a) providing a cell according to claim 7;
(a1) adding the compound to the cells of step (a);
(a2) adding a translocation stimulus to the cells of step (a1):
(b) add an interaction stimulus;
(c) determine if complementation partner A and complementation partner B has complemented;
less complementation compared to a system omitting step (a1) being indicative that the compound prevents translocation of the protein.

73. A kit for detecting antigens comprising:
(a) a first antibody that binds to the antigen;
(b) a first conjugate comprising a protein binding to the first antibody and the N-terminal of a complementation protein;
(c) a second conjugate comprising a protein binding to the first antibody and the C-terminal of the complementation protein;
whereby binding of the first and second conjugate to the first antibody will bring the two terminals of the complementation protein so close that a functional protein is formed.

74. A method for determining caspase activity in a cell comprising at least the steps of
(a) providing a cell comprising at least:
a first conjugate comprising the first terminal of a complementation protein, interaction partner A, a nuclear localisation sequence (NLS), a Valine-Alanine-Aspartate (VAD) sequence and an anchor protein;
a second conjugate comprising the second terminal of a complementation protein, interaction partner B and an anchor protein;
wherein the anchor protein of the first conjugate is in a different cellular location from the nucleus, and the anchor protein of the second conjugate is in the nucleus and
wherein interaction partner A, the NLS and the first terminal of the complementation protein are all located on the same side of the VAD sequence and the anchor protein is located on the other side of the NLS;
(b) determine if the complementation protein has complemented
complementation being indicative that caspase has cleaved the first conjugate and the free part has translocated to the nucleus and partner A and complementation partner B has complemented.

75. A nucleic acid encoding any of the conjugates, fragments or fusions proteins.
